(19) United States
(12) Patent Application Publication
(10) Pub. No.: US 2023/0233576 A1
(43) Pub. Date: Jul. 27, 2023

(54) ECLITASERTIB FOR USE IN TREATING CONDITIONS INVOLVING SYSTEMIC HYPERINFLAMMATORY RESPONSE

(71) Applicant: Genzyme Corporation, Cambridge, MA (US)

(72) Inventors: John CAMINIS, Cambridge, MA (US); Peter FLORIAN, Bad Orb (DE); Keith HARRIS, Cambridge, MA (US); Dimitry OFENGEIM, Cambridge, MA (US); Sabine SCHNYDER, Cambridge, MA (US); Heribert STAUDINGER, Cambridge, MA (US); Matija ZELIC, Cambridge, MA (US)

(21) Appl. No.: 17/918,973

(22) PCT Filed: Apr. 16, 2021
(86) PCT No.: PCT/US2021/027593

§ 371 Date: Oct. 14, 2022

Related U.S. Application Data
(60) Provisional application No. 63/011,874, filed on Apr. 17, 2020.

(51) Int. Cl. A61K 31/553; A61P 31/14; A61K 31/573; A61K 31/675; A61K 31/4706; A61K 31/215; A61K 31/522; A61K 31/7056; A61K 31/513; A61K 31/427; A61K 31/4965; A61K 31/635; A61K 31/196

(52) U.S. Cl. CPC A61K 31/553; A61P 31/14; A61K 31/573; A61K 31/675; A61K 31/4706; A61K 31/215; A61K 31/522; A61K 31/7056; A61K 31/513; A61K 31/427; A61K 31/4965; A61K 31/635; A61K 31/196

(57) ABSTRACT

This disclosure relates to the field of therapeutic protein kinase inhibitors, in particular receptor-interacting protein kinase 1 (“RIPK1”) inhibitor for treatment of subjects with conditions involving systemic hyperinflammatory responses, such as Cytokine Release Syndrome (CRS), or Systemic Inflammatory Response Syndrome (SIRS), sepsis, organ damage, or hyperinflammatory state associated with infectious diseases.

This application claims priority to U.S. Provisional Application No. 63/011,874, filed Apr. 17, 2020, the contents of which is incorporated herein by reference for all purposes.

### INTRODUCTION AND SUMMARY

This disclosure relates to the field of protein kinase inhibitors, in particular receptor-interacting protein kinase 1 (RIPK1) inhibitor compounds, to treat conditions involving systemic hyperinflammatory responses, such as Cytokine Release Syndrome (CRS), or Systemic Inflammatory Response Syndrome (SIRS), sepsis, organ damage, or hyperinflammatory state associated with infectious diseases such as coronavirus infection.

RIPK1 is a key regulator of inflammation, apoptosis and necroptosis. RIPK1 has an important role in modulating inflammatory responses mediated by nuclear-factor kappa-light chain enhancer of activated B cells (NF-κB). Research has shown that its kinase activity controls necroptosis, a form of programmed cell death, which was traditionally thought to be passive and unregulated, and is characterized by a unique morphology. Necroptosis is dependent on the sequential activation of RIPK 1 and 3, ultimately leading to MLKL (Mixed Lineage Kinase domain-Like pseudokinase) activation, translocation to cellular membranes and death by membrane rupture. RIPK1 is also part of a pro-apoptotic complex, indicating its activity in regulating apoptosis.

RIPK1 is subject to complex and intricate regulatory mechanisms, including ubiquitylation, deubiquitylation and phosphorylation. These regulatory events collectively determine whether a cell will survive and activate an inflammatory response, or die through apoptosis or necroptosis. Dysregulation of RIPK1 signaling can lead to excessive inflammation or cell death, and conversely, research has shown that inhibition of RIPK1 can be an effective therapy for diseases involving inflammation or cell death.

RIPK1 kinase-driven inflammation and cell death have been suggested as contributing factors to TNFα-induced systemic inflammatory response syndrome (SIRS). Zelic M. et al. (2018) J. Clin Invest. 128(5): 2064-75. In addition to exacerbated inflammatory signaling, RIPK1 kinase inhibition is also suggested to suppress vascular system dysfunction and endothelial/epithelial cell damage, ultermately leading to organ damage. Id. Accordingly, RIPK1 inhibition may play a role in ameoliating or treating SIRS, organ damage, and sepsis-related inflammation.

The recent emergence of COVID-19 coronavirus infection as a major public health threat has additionally required a need for novel therapies to treat or prevent the condition.

Accordingly, the following embodiments are provided.

Embodiment 1 is a method of treating a subject at risk of or having Cytokine Release Syndrome (CRS), comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Embodiment 2 is a method of treating a subject in a hyperinflammatory state, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Embodiment 3 is a method of treating a subject at risk of or having Systemic Inflammatory Response Syndrome (SIRS), comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Embodiment 4 is a method of reducing inflammation in a subject at risk of or having CRS or SIRS, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Embodiment 5 is a method of reducing organ damage in a subject at risk of or having CRS or SIRS, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Embodiment 6 is a method of reducing sepsis-related inflammation and organ injury in a subject, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Embodiment 7 is a method of treating a subject having influenza-like illness, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Embodiment 8 is a method of reducing symptoms related to coronavirus infection, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Embodiment 9 is the method of embodiment 8, wherein the coronavirus infection is by COVID-19/2019-nCoV/SARS-CoV-2, SARS-CoV, and/or MERS-CoV.

Embodiment 10 is the method of any one of embodiments 1-9, wherein the RIPK1 inhibitor is (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt thereof.

Embodiment 11 is the method of any one of embodiments 1-10, wherein a dose of about 5 mg to about 1000 mg of the RIPK1 inhibitor is administered.

Embodiment 12 is the method of embodiment 11, wherein the dose is 400 mg.

Embodiment 13 is the method of embodiment 11, wherein the dose is 600 mg.

Embodiment 14 is the method of embodiment 11, wherein the dose is 800 mg.

Embodiment 15 is the method of embodiment 11, wherein the dose is 1000 mg.

Embodiment 16 is the method of any one of embodiments 1-15, wherein the RIPK1 inhibitor is administered daily.

Embodiment 17 is the method of any one of embodiments 1-16, wherein the RIPK1 inhibitor is administered in conjunction with antiviral therapy.

Embodiment 18 is the method of embodiment 17, wherein the antiviral therapy is chosen from remdesivir, hydroxychloroquinine, galidesivir, oseltamivir, paramivir, zanamivir, ganciclovir, acyclovir, ribavirin, lopinavir, ritonavir, favipiravir, darunavir or a combination thereof.

Embodiment 19 is the method of any one of embodiments 1-16, wherein the RIPK1 inhibitor is administered in conjunction with a corticosteroid treatment.

Embodiment 20 is the method of embodiment 18, wherein the corticosteroid treatment is chosen from dexamethasone, betamethasone, prednisone, prednisolone, methylprednisolone, cortisone, hydrocortisone, triamcinolone, or ethamethasoneb or a combination thereof.

Embodiment 21 is the method of any one of embodiments 1-20, wherein the RIPK1 inhibitor is administered orally.

Embodiment 22 is the method of any one of embodiments 1-20, wherein the RIPK1 inhibitor is administered via gastric feeding tube.

Embodiment 23 is the method of any one of embodiments 1-22, wherein the condition of the subject comprises a systemic hyperinflammatory response.

Embodiment 24 is the method of embodiment 24, wherein the systemic hyperinflammatory response is shown by increase in CRP, decrease in leukocyte number, change in neutrophil number, decrease in neutrophil to lymphocyte ratio, and/or increase in IL-6.

Embodiment 25 is the method of any one of embodiments 1-22, wherein the condition of the subject indicates innate immunity activation.

Embodiment 26 is the method of embodiment 25, wherein innate immunity activation is shown by increase in CRP, change in neutrophil number, and/or increase in IL-6.

Embodiment 27 is a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in treating a subject at risk of or having Cytokine Release Syndrome (CRS) or Inflammatory Response Syndrome (SIRS).

Embodiment 28 is a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in treating a subject in a hyperinflammatory state.

Embodiment 29 is a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in reducing inflammation or organ damage in a subject at risk of or having CRS or SIRS.

Embodiment 30 is a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in reducing sepsis-related inflammation or organ damage in a subject.

Embodiment 31 is a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in treating a subject having influenza-like illness.

### BRIEF DESCRIPTION OF DRAWINGS

FIG. 1 shows an exemplary overall design of treatment with the exemplary RIPK1 inhibitor for treating a subject having a coronavirus infection.

FIG. 2 shows a summary plot of point estimates of the relative change in CRP from baseline (geometric means) with 90% confidence interval over treatment period by treatment arm in the Efficacy population according to Example 2. The linear mixed effects model on log (relative change in CRP) includes baseline log-CRP, visit, treatment group and visit-by-treatment group interaction as fixed effects and sites as a random effect. Repeated measures within participants are modeled with an unstructured residual covariance matrix. Point estimate obtained is back-transformed to original scale by exponentiation (point estimate displayed). Point estimate is a value lower than 1 indicates a decrease from baseline. Missing values for the relative change from baseline in CRP for Days 3,5,7,15 were replaced following the LOCF approach. When several values are available on a day, the last available and evaluable value is considered for the analysis.

FIG. 3 shows Kaplan-Meier curves for time to 50% improvement in CRP levels in the Efficacy population according to Example 2. 50% decrease relative to baseline CRP level is considered as event. Event times for participants not meeting this criterion will be censored at the last observation time point. For patients who have died during the study without experiencing the event, the last observation collected is carried forward to the longest duration of follow-up for any patient plus 1 day.

FIG. 4 shows a boxplot of raw value in CRP level over time in the Efficacy population according to Example 2. For the boxplots shown in all of the figures provided herein, the solid diamond corresponds to the group arithmetic mean; the horizontal line in the box interior represents the group median; the length of the box represents the interquartile range (the distance between the 25th and 75th percentiles); and the other symbols correspond to participant values.

FIG. 5 shows Kaplan-Meier curves for time to improvement of oxygenation (SpO₂) in the Efficacy population according to Example 2. Presence of SpO₂>=92% without use of any supplemental oxygen device on two consecutive days or at day of discharge is considered as event. Event times for participants not meeting this criterion will be censored at the last observation time point. For patients who have died during the study without experiencing the event, the last observation collected is carried forward to the longest duration of follow-up for any patient plus 1 day.

FIG. 6 shows a summary plot of point estimates of the absolute change in SpO₂/FiO₂ ratio from baseline with 90% confidence interval over treatment period by treatment arm in the Efficacy population according to Example 2. The linear mixed effects model on change in SpO₂/FiO₂ ratio includes baseline value, visit, treatment group and visit-by-treatment group interaction as fixed effects and sites as a random effect. Repeated measures within participants are modeled with an unstructured residual covariance matrix. Point estimate is a positive value indicates an improvement from baseline in SpO₂/FiO₂ ratio. Missing values were replaced following the LOCF approach. When several values are available on a day, the most severe measurement of the day based on the SpO₂/FiO₂ ratio is considered for the analysis.

FIG. 7 shows a boxplot of SpO₂/FiO₂ ratio raw value over time in the Efficacy population according to Example 2.

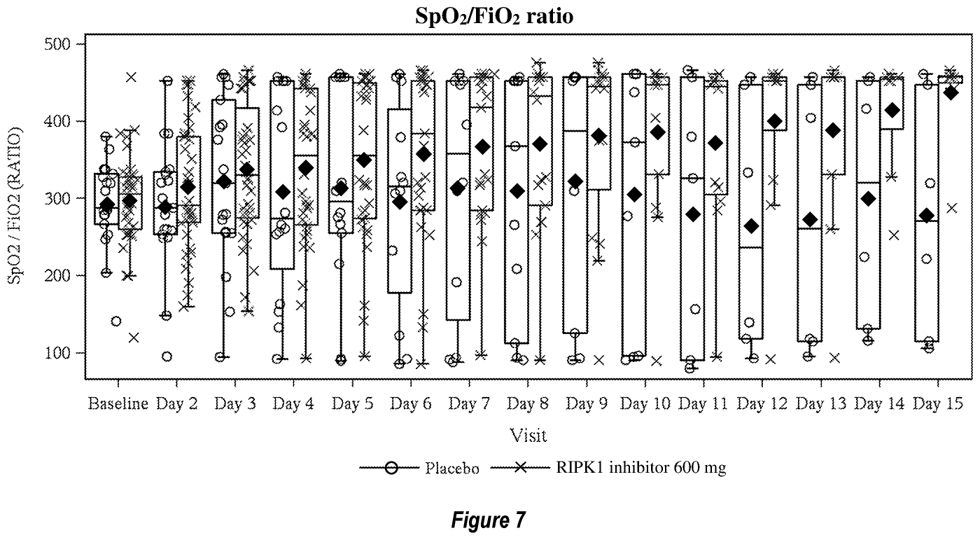

FIG. 8 shows a stacked bar plot of the percentage of participants per 7-point clinical scale category over treatment period in the Efficacy population according to Example 2. 1=Death, 2=Hospitalized, on invasive mechanical ventilation or ECMO, 3=Hospitalized, on non-invasive ventilation or high flow oxygen devices, 4=Hospitalized, requiring supplemental oxygen, 5=Hospitalized, not requiring supplemental oxygen—requiring ongoing medical care (COVID-19 related or otherwise), 6=Hospitalized, not requiring supplemental oxygen—no longer requires ongoing medical care, 7=Not hospitalized. When several values for 7-point clinical scale are available on a day, the last available and evaluable value is considered for the analysis. Missing values for 7-point clinical scale are replaced following the LOCF approach. For participants who are discharged from hospital before Day 15, if no data available after discharge until Day 15 for the 7-point clinical scale, the participant is considered as “7—not hospitalized”. For participants who died before Day 15, the participant is considered as “1—death” after death until Day 15 for the 7-point clinical scale. On the day of hospital discharge due to recovery, the value for 7-point clinical scale is defined as “7—not hospitalized” by default.

FIG. 9 shows Kaplan-Meier curves for time to improvement in 7-point clinical scale by at least two points in the Efficacy population according to Example 2. An improvement of at least 2 points in category of 7-point clinical scale from baseline is considered as event. Event times for participants not meeting this criterion will be censored at the last observation time point. For patients who have died during the study without experiencing the event, the last observation collected is carried forward to the longest duration of follow-up for any patient plus 1 day. On the day of hospital discharge due to recovery, the value for 7-point clinical scale is defined as “7—not hospitalized” by default.

FIG. 10 shows a boxplot of Chemokine (C-X-C Motif) Ligand 10 (pg/mL) with LOCF imputation in the Safety population according to Example 2. For FIGS. 10-13, baseline is defined as the D1 predose assessment value; values below LLOQ are replaced by LLOQ/2; outlier values higher than Q3+3 IQR are imputed by Q3+3 IQR; missing data are imputed by Last Observation Carried Forward (LOCF) method if at least a baseline and a post-baseline value were available; and unscheduled and discharge before Day 15 (treatment period) visits are re-allocated to study visits according to their study day.

FIG. 11 shows a boxplot of Interferon Gamma (pg/mL) with LOCF imputation in the Safety population according to Example 2.

FIG. 12 shows a boxplot of Interleukin 10 (pg/mL) with LOCF imputation in the Safety population according to Example 2.

FIG. 13 shows a boxplot of raw value of Interleukin 6 (pg/mL) with LOCF imputation in the Safety population according to Example 2.

FIG. 14 shows a boxplot of raw value of D-Dimer over time in the Efficacy population according to Example 2. For FIGS. 14-19, Baseline is defined as the last available and evaluable value before and closest to the first dose of the Investigational Medicinal Product administration.

FIG. 15 shows a boxplot of raw value of leukocytes over time in the Efficacy population according to Example 2.

FIG. 16 shows a boxplot of raw value of ferritin over time in the Efficacy population according to Example 2.

FIG. 17 shows a boxplot of raw value of lymphocytes over time in the Efficacy population according to Example 2.

FIG. 18 shows a boxplot of raw value of Neutrophils/Lymphocytes over time in the Efficacy population according to Example 2.

FIG. 19 shows a boxplot of raw value of Lactate Dehydrogenase (LDH) over time in the Efficacy population according to Example 2.

FIG. 20 shows a boxplot of Eotaxin-1 (pg/mL) with LOCF imputation in the the Safety population according to Example 2. For FIGS. 20-28, baseline is defined as the D1 predose assessment value; values below LLOQ are replaced by LLOQ/2; outlier values higher than Q3+3 IQR are imputed by Q3+3 IQR; missing data are imputed by Last Observation Carried Forward (LOCF) method if at least a baseline and a post-baseline value were available; and unscheduled and discharge before Day 15 (treatment period) visits are re-allocated to study visits according to their study day.

FIG. 21 shows a boxplot of Chemokine (C-C Motif) Ligand 17 (pg/mL) with LOCF imputation in the Safety population according to Example 2.

FIG. 22 shows a boxplot of Interleukin 8—Cytokines (pg/mL) with LOCF imputation in the Safety population according to Example 2.

FIG. 23 shows a boxplot of Macrophage-Derived Chemokine (pg/mL) with LOCF imputation in the Safety population according to Example 2.

FIG. 24 shows a boxplot of Monocyte Chemotactic Protein 1 (pg/mL) with LOCF imputation in the Safety population according to Example 2.

FIG. 25 shows a boxplot of Tumor Necrosis Factor alpha (pg/mL) with LOCF imputation in the Safety population according to Example 2.

FIG. 26 shows a boxplot of Macrophage Inflammatory Protein 1 Beta (pg/mL) with LOCF imputation in the Safety population according to Example 2.

FIG. 27 shows a boxplot of Chemokine (C-C Motif) Ligand 13 (pg/mL) with LOCF imputation in the Safety population according to Example 2.

FIG. 28 shows a boxplot of Ratio of Interleukin 6 and Interleukin 10 (RATIO) with LOCF imputation in the Safety population according to Example 2.

### DETAILED DESCRIPTION

The present disclosure relates to treating conditions involving systemic hyperinflammatory responses, such as cytokine release syndrome (CRS), systemic inflammatory response syndrome (SIRS), organ damage, sepsis, and hyperinflammatory state associated with infectious diseases such as coronavirus infection, with a RIPK1 inhibitor compound, e.g., as a rescue therapy, to attenuate the exaggerated immune response caused by the viral infection and the accompanying over-expressed excessive inflammatory response. Without intending to be limited to a particular mechanism, administration of a RIPK1 inhibitor compound is believed to inhibit or reduce cell death (necroptosis) and prevent further damage to surrounding cells, therefore reducing the degree of inflammation caused by, e.g., infectious diseases such as a coronavirus infection.

Reference will now be made in detail to certain embodiments, examples of which are illustrated in the accompanying drawings.

While this disclosure provides certain illustrated embodiments, it will be understood that they are not intended to limit the invention to those embodiments. On the contrary, the invention is intended to cover all alternatives, modifications, and equivalents, which may be included within the disclosure as defined by the appended claims.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments.

On the contrary, the present teachings encompass various alternatives, modifications, and equivalents, as will be appreciated by those of skill in the art.

### I. Definitions

Unless otherwise stated, the following terms used in the specification and claims are defined for the purposes of this disclosure and have the following meaning(s):

A “pharmaceutically acceptable carrier” or a “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development of the disease or its clinical symptoms; or
(3) relieving the disease, e.g., causing regression of the disease or its clinical symptoms.

“Optional” or “optionally” means that the subsequently described event or circumstance may but need not occur, and that the description includes instances where the event or circumstance occurs and instances in which it does not.

A “therapeutically effective amount” means the amount of the RIPK1 inhibitor compound, that, when administered to a mammal for treating a disease, is sufficient to effect such treatment for the disease. The “therapeutically effective amount” will vary depending on the compound, the disease and its severity and the age, weight, etc., of the mammal to be treated.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

As used herein, “cytokine release syndrome,” “cytokine syndrome,” or CRS refers to a systemic inflammatory response caused by a large, rapid release of cytokines into the blood from immune cells and can be triggered by a variety of factors such as infections, drugs, or immunotherapy. Symptoms of cytokine release syndrome include, but are not limited to, fever, nausea, headache, rash, rapid heartbeat, low blood pressure, and trouble breathing. The reaction may be severe or life-threatening.

As used herein, “Systemic inflammatory response syndrome” or “SIRS”, also known as acute inflammatory syndrome, is an inflammatory condition affecting the whole body. SIRS is the body's response to an infectious or noninfectious assault. SIRS is related to systemic inflammation, organ dysfunction, and organ failure, and is a subset of cytokine storm in which there is an abnormal regulation of various cytokines. It is also closely related to sepsis, in which patients satisfy criteria for SIRS and have a suspected or proven infection. Complications of SIRS may include acute kidney injury, shock, and multiple organ dysfunction syndrome. Causes of SIRS may include microbial infections, malaria, trauma, burns, pancreatitis, ischemia, hemorrhage, complications of surgery, adrenal insufficiency, pulmonary embolism, aortic aneurysm, cardiac tamponade, anaphylaxis, and drug overdose.

As used herein, sepsis is an inflammatory immune response triggered by an infection. It is a life-threatening condition that is present when the body causes injury to its own tissues and organs while responding to an infection. The infection may be caused by bacteria (most common), fungus, virus, and protozoans. Symptoms of sepsis may include fever, increased heart rate, low blood pressure, increased breathing rate, and confusion.

“Coronavirus infection” means infection by a coronavirus including alpha- and beta-coronaviruses, including, 2019-nCoV/SARS-CoV-2 (also known COVID-19), SARS-CoV, HCoV, and/or MERS-CoV. Nonlimiting examples of types of coronavirus infection include COVID-19, SARS, and MERS.

The “RIPK1 Inhibitor” refers to (S)-5-benzyl-N-(5-methyl-4-oxo-2,3,4,5-tetrahydropyrido[3,2-1][1,4]oxazepin-3-yl)-4H-1,2,4-triazole-3-carboxamide, having the following structure:

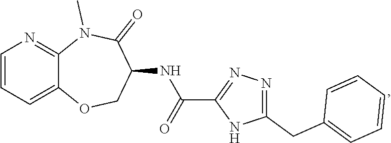 [SMILES: CN1C(=O)[C@@H](NC(=O)c2nnc(Cc3ccccc3)[nH]2)COc2cccnc21]

and/or a pharmaceutically acceptable salt, tautomer, stereoisomer or mixture of stereoisomers thereof.

It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims.)

Before describing the present teachings in detail, it is to be understood that the disclosure is not limited to specific compositions or process steps, as such may vary.

### II. RIPK1 Inhibitor Compounds

In some embodiments, a method of treating a subject at risk of or having cytokine release syndrome (CRS) is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof. In some embodiments, the CRS is in its early stages. In some embodiments, the CRS is at or near its peak.

In some embodiments, a method of treating a subject at risk of or having Systemic Inflammatory Response Syndrome (SIRS) is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof. In some embodiments, the SIRS is in its early stages. In some embodiments, the SIRS is at or near its peak.

In some embodiments, a method of treating a subject in a hyperinflammatory state is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin yl)-4H-1,2,4-triazole-3-carboxamide, and/or a pharmaceutically acceptable salt, tautomer, stereoisomer or mixture of stereoisomers thereof. In some embodiments, the hyperinflammatory state is shown by an increase in CRP, decrease in leukocyte number, a change in neutrophile number (blood neutrophilia or blood neutropenia), decrease in neutrophil-to-lymphocyte ratio, and/or an increase in IL-6.

In some embodiments, a method of reducing inflammation in a subject at risk of or having CRS is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

In some embodiments, a method of reducing inflammation in a subject at risk of or having SIRS is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

In some embodiments, a method of reducing organ damage in a subject in a hyperinflammatory state, including in a subject at risk of or having CRS is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

In some embodiments, a method of reducing organ damage in a subject in a hyperinflammatory state, including in a subject in a subject at risk of or having SIRS is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

In some embodiments, a method of reducing sepsis-related inflammation and/or organ injury in a subject is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

In some embodiments, a method of treating a subject having influenza-like illness is provided, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof. Non-limiting examples of influenza-like illness or symptoms are fever, cough, sputum production, wheezing, difficulty breathing, nasal congestion, rhinorrhea, pharyngitis, otitis, vomiting, diarrhea, sore throat, chills (shivering), tiredness (fatigue), headache, and myalgia (muscle aches).

In an embodiment, a method of treating coronavirus infection is provided comprising administering to a subject in need thereof a RIPK1 inhibitor such as (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt thereof. In another embodiment, a method of reducing symptoms related to coronavirus infection, includes administering to a subject in need thereof a RIPK1 inhibitor such as (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt thereof. In an embodiment, the subject exhibits symptoms characteristic of cytokine release syndrome (“CRS”; also known as “cytokine storm”).

In an embodiment, a method of treating a subject diagnosed with the effects of CRS includes administration of a RIPK1 inhibitor such as (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt thereof. In some embodiments, the CRS is in its early stages. In some embodiments, the CRS is at or near its peak.

In an embodiment, the condition of the subject indicates dysfunctional immune response. In an embodiment, the dysfunctional immune response is CRS. In another embodiment, innate immunity activation in the subject is shown by an increase in C-reactive protein (“CRP”), decrease in neutrophil number, and/or an increase in IL-6.

In some embodiment, the condition of the subject comprises a systemic hyperinflammation response. In some embodiments, the systemic hyperinflammation response is shown by an increase in CRP, decrease in leukocyte, a change in neutrophile number (blood neutrophilia or blood neutropenia), decrease in neutrophil-to-lymphocyte ratio, and/or an increase in IL-6.

In other embodiments, a dose of about 5 mg to about 1000 mg of the RIPK1 inhibitor, e.g., 5, 15, 20, 50, 60, 100, 150, 200, 300, 400, 600, 800 or 1000 mg, is administered.

In some embodiments, a dose of about 400 mg to about 1000 mg of the RIPK1 inhibitor, e.g., 400, 500, 600, 700, 800, 900, or 1000 mg is administered. In some embodiments, a dose of about 400 mg is administered. In some embodiments, a dose of about 500 mg is administered. In some embodiments, a dose of about 600 mg is administered. In some embodiments, a dose of about 800 mg is administered. In some embodiments, a dose of about 1000 mg is administered.

In an embodiment, the RIPK1 inhibitor is administered in conjunction with antiviral therapy, such as remdesivir, hydroxychloroquinine, galidesivir, oseltamivir, paramivir, zanamivir, ganciclovir, acyclovir, ribavirin, lopinavir, ritonavir, favipiravir, darunavir, or a combination thereof.

In some embodiments, the RIPK1 inhibitor is administered in conjunction with a steroid, such as a corticosteroid. In some embodiments, the corticosteroid is dexamethasone, betamethasone, prednisone, prednisolone, methylprednisolone, cortisone, hydrocortisone, triamcinolone, or ethamethasone, or a pharmaceutically acceptable salt thereof.

The RIPK1 Inhibitor can be prepared according to the methods and schemes described in, e.g., U.S. Pat. No. 9,896,458, in particular the content of Example 42, which is incorporated herein by reference.

Several preclinical studies have demonstrated a role for RIPK1/RIPK3 activation in the pathogenesis of severe shock or sepsis and inflammatory diseases. Importantly, RIPK1 kinase-dead (KD) and RIPK3 knockout (KO) mice have been shown to be resistant to lethal Systemic Inflammatory Response Syndrome (SIRS) induced by TNFα. Recent clinical data suggest a role for necroptosis activation during sepsis, with RIPK3 up-regulation in the plasma correlating with death of critically ill patients. However, MLKL KO mice are more susceptible to TNFα-induced shock than RIPK1 KD or RIPK3 KO mice, suggesting that both RIPK1 kinase-driven inflammation and cell death are key contributing factors to TNFα-induced SIRS. The RIPK1 Inhibitor was studied in an acute mouse model of SIRS. Similar to published data we have found that SIRS induction is dose-dependently blocked and at the highest dose completely abolished. There is also rationale that vascular permeability and endothelial dysfunction contribute to SIRS/shock and lethality. We have demonstrated that TNFα alone induced shock in the SIRS mouse model which is rescued by genetic RIPK1 kinase inhibition specifically in non-hematopoietic cells by means of bone marrow transplantation. Importantly, non-hematopoietic kinase inactive cells afforded protection from TNFα-induced vascular hyperpermeability and coagulation and liver endothelial cell necroptosis. These data indicate that RIPK1 kinase inhibition may suppress vascular system dysfunction and endothelial/epithelial cell damage in addition to exacerbated inflammatory signaling. Additional clinical evidence for the role of RIPK1 in driving systemic inflammation comes from evidence in a rare population of patients that have a mutation in RIPK1 that blocks caspase-mediated cleavage and leads to hyperactivation of this kinase. These patients have periodic fevers with coinciding elevations of cytokines including IL-6 and elevated levels of pRIPK1 in their PBMCs. Patient-derived cells are responsive to RIPK1 kinase inhibition, and some patients are responsive to anti-IL-6 therapy.

Accordingly, in some embodiments, administration of the RIPK1 inhibitor reduces the effects of SIRS. In some embodiments, administration of the RIPK1 inhibitor reduces inflammation associated with SIRS. In some embodiments, administration of the RIPK1 inhibitor reduces organ damage associated with SIRS. In some embodiments, administration of the RIPK1 inhibitor alleviates a hyperinflammation state. In some embodiments, administration of the RIPK1 inhibitor treats or reduces sepsis-related inflammation or organ injury.

In ‘Pathogenic human coronavirus infections: causes and consequences of cytokine storm and immunopathology,’ Channappanavar and Perlam state: “In vitro studies after the previous SARS-CoV outbreak show that infection of human dendritic cells with SARS-CoV induces low-level expression of antiviral cytokines IFN-αβ, moderate up-regulation of pro-inflammatory cytokines TNF and IL-6, and a significant up-regulation of inflammatory chemokines CCL3 (also known as MIP1α), CCL5, CCL2, and CXCL10. Similarly, SARS-CoV-infected macrophages show delayed but elevated levels of IFN and other pro-inflammatory cytokines. SARS-CoV-infected airway epithelial cells (AECs) also produce large amounts of CCL3, CCL5, CCL2, and CXCL10. The delayed but excessive production of these cytokines and chemokines is thought to induce a dysregulated innate immune response to SARS-CoV infection. High serum levels of pro-inflammatory cytokines (IFN-γ, IL-1, IL-6, IL-12, and TGFβ) and chemokines (CCL2, CXCL10, CXCL9, and IL-8) were found in SARS patients with severe disease compared to individuals with uncomplicated SARS. Conversely, SARS patients with severe disease had very low levels of the anti-inflammatory cytokine, IL-10. In addition to pro-inflammatory cytokines and chemokines, individuals with lethal SARS showed elevated levels of IFN (IFN-α and IFN-γ) and IFN-stimulated genes (ISGs) (CXCL10 and CCL-2) compared to healthy controls or individuals with mild-moderate disease. These results were the first to suggest a possible role for IFNs and ISGs in the immunopathogenesis of SARS in humans. Thus, it appears from these studies that dysregulated and/or exaggerated cytokine and chemokine responses by SARS-CoV-infected AECs, DCs, and macrophages could play an important role in SARS pathogenesis.”

Since RIPK1 kinase activity regulates the execution of cell death in innate immune cells after interferon receptor stimulation, and inhibition of RIPK1 has been shown to decrease interferon response in vitro in macrophages and reducing production of, e.g., CCL3 (MIP1α), the methods of the invention may be used to stifle the exaggerated antiviral response mounted by the innate immune system by a broader mechanism than IL-6-pathway inhibition.

In some embodiments, administration of the RIPK1 inhibitor reduces the effects of cytokine release syndrome (“CRS”; also known as “cytokine storm.”) CRS, as related to infectious diseases, is the excessive or uncontrolled release of proinflammatory cytokines in response to the infection. CRS is characterized by increased plasma concentrations of interleukins, interferons, chemokines, colony-stimulating factors (CSFs), and tumor necrosis factors, e.g., IL-6, IFNγ, MCP-1, IL-10 and TNFα.

In some embodiments, the infectious diseases characterized by CRS is an infection by a coronavirus including 2019-nCoV/SARS-CoV-2, SARS-CoV, and MERS-CoV. In some embodiments, the subject has severe or critical disease. In some embodiments, the subject has multi-organ dysfunction. In some embodiments, the subject has pneumonia and fever.

In some embodiments, the CRS is characterized by increased plasma concentrations of one or more cytokines selected from interleukins, interferons, chemokines, CSFs, and TNFα. In some embodiments, the interleukins are selected from IL-la, IL-1(3, IL-1RA, IL-2, IL-6, IL-7, IL-8, IL-9, IL-10, and IL-18. In some embodiments, the interferons are selected from IFNα, IFNβ, IFNγ, IFN-λ1, IFV-λ2, and INF-λ3. In some embodiments, the chemokines are selected from CXCR3 ligands, CXCL8, CXCL9, CXCL10, CXCL11, CCL2 (monocyte chemoattractant protein 1 [MCP-1]), CCL3, CCL4, and CCL11 (eotaxin). In some embodiments, the CSFs are selected from granulocyte-macrophage colony-stimulating factor (GM-CSF), macrophage colony-stimulating factor (M-CSF), and granulocyte colony-stimulating factor (G-CSF).

In some embodiments, the CRS is characterized by increased plasma concentrations of interleukins 2, 7, and 10, granulocyte-colony stimulating factor, interferon-γ-inducible protein 10, monocyte chemoattractant protein 1, macrophage inflammatory protein 1 alpha, and/or TNFα. In some embodiments, the CRS is characterized by increased plasma concentrations of platelet-derived growth factor (PDGF). In some embodiments, the CRS is characterized by increased plasma concentrations of vascular endothelial growth factor (VEGF). In some embodiments, the CRS is characterized by increased plasma concentrations of basic fibroblast growth factor (bFGF). In some embodiments, the subject in need thereof is suffering from one or more symptoms selected from pneumonia, bronchitis, fever, coughing, productive cough, runny nose, sneezing, breathlessness, sharp or stabbing chest pain during deep breaths, chills, exacerbated asthma, increased rate of breathing, acute respiratory distress syndrome (ARDS), RNAaemia (detectable RNA in the bloodstream), acute cardiac injury, shock, myalgia, fatigue, sputum production, rusty colored sputum, bloody sputum, swelling of lymph nodes, middle ear infection, joint pain, wheezing, headache, hemoptysis, diarrhea, dyspnea, redness, swelling or edema, pain, loss of function, organ dysfunction, multi-organ system failure, acute kidney injury, confusion, malnutrition, blue-tinged skin, sepsis, hypotension, hypertension, hypothermia, hypoxemia, leukocytosis, leukopenia, lymphopenia, thrombocytopenia, nasal congestion, sore throat, unwillingness to drink, convulsions, ongoing vomiting, extremes of temperature, decreased level of consciousness, abdominal pain, and secondary infection.

In some embodiments, the subject in need thereof has pulmonary complications characterized by abnormalities in chest CT images. In some embodiments, the subject in need thereof exhibits ground-glass opacity and subsegmental areas of consolidation in chest CT images. In some embodiments, the subject in need thereof exhibits multiple lobular and subsegmental areas of consolidation in chest CT images. In some embodiments, the subject in need thereof exhibits bilateral involvement of ground-glass opacity and subsegmental areas of consolidation in chest CT images. In some embodiments, the subject in need thereof exhibits bilateral involvement of multiple lobular and subsegmental areas of consolidation in chest CT images.

In some embodiments, the subject in need thereof has elevated levels, relative to a healthy subject, of aspartate aminotransferase. In some embodiments, the subject in need thereof has elevated levels, relative to a healthy subject, of D-dimer. In some embodiments, the subject in need thereof has elevated levels, relative to a healthy subject, of hypersensitive troponin I (hs-cTnl). In some embodiments, the subject in need thereof has elevated levels, relative to a healthy subject, of procalcitonin levels, e.g., a procalcitonin level greater than 0.5 ng/mL. In some embodiments, the subject in need thereof has an elevated prothrombin time relative to a healthy subject.

In some embodiments, the subject in need thereof is an adult. An adult is a human subject greater than, or equal to, 18 years of age. In some embodiments, the subject in need thereof is greater than or equal to 18 years of age and less than or equal to 59 years of age. In some embodiments, the subject in need thereof is 60 years of age or older.

In some embodiments, the subject in need thereof is younger than 18 years of age.

In some embodiments, the subject in need thereof is greater than, or equal to, 12 years of age.

In some embodiments, the subject in need thereof has a long-term or pre-existing medical condition, for example, but not limited to, heart disease, lung disease, diabetes, cancer and/or high blood pressure.

In some embodiments, the subject in need thereof has a weakened immune system.

In some embodiments, administration of the RIPK1 Inhibitor treats or ameliorates one or more symptoms of pneumonia, bronchitis, fever, coughing, productive cough, runny nose, sneezing, breathlessness, sharp or stabbing chest pain during deep breaths, chills, exacerbated asthma, increased rate of breathing, acute respiratory distress syndrome (ARDS), RNAaemia (detectable RNA in the bloodstream), acute cardiac injury, shock, myalgia, fatigue, sputum production, rusty colored sputum, bloody sputum, swelling of lymph nodes, middle ear infection, joint pain, wheezing, headache, hemoptysis, diarrhea, dyspnea, redness, swelling or edema, pain, loss of function, organ dysfunction, multi-organ system failure, acute kidney injury, confusion, malnutrition, blue-tinged skin, sepsis, hypotension, hypertension, hypothermia, hypoxemia, leukocytosis, leukopenia, lymphopenia, thrombocytopenia, nasal congestion, sore throat, unwillingness to drink, convulsions, ongoing vomiting, extremes of temperature, decreased level of consciousness, abdominal pain, and/or secondary infection.

In some embodiments, administration of the RIPK1 Inhibitor reduces levels of aspartate aminotransferase in a subject. In some embodiments, administration of the RIPK1 Inhibitor reduces levels of D-dimer in a subject. In some embodiments, administration of the RIPK1 Inhibitor reduces levels of hypersensitive troponin I (hs-cTnl) in a subject. In some embodiments, administration of the RIPK1 Inhibitor reduces procalcitonin levels in a subject. In some embodiments, administration of the RIPK1 Inhibitor reduces prothrombin time in a subject.

In some embodiments, administration of the RIPK1 Inhibitor reduces and/or eliminates one or more pulmonary complications characterized by abnormalities in chest CT images. In some embodiments, administration of the RIPK1 Inhibitor reduces the incidence of death in a subject infected with an infectious disease characterized by CRS. In some embodiments, administration of the RIPK1 Inhibitor reduces and/or eliminates the need for mechanical ventilation, supplemental oxygen and/or hospitalization in the subject.

In some embodiments, administration of the RIPK1 Inhibitor reduces influenza-like illness such as fever, cough, sputum production, wheezing, difficulty breathing, nasal congestion, rhinorrhea, pharyngitis, otitis, vomiting, diarrhea, sore throat, chills (shivering), tiredness (fatigue), headache, and myalgia (muscle aches). In some embodiments, the influenza-like illness is the occurrence of fever greater than or equal to 38° C. for at least 24 hours. In some embodiments, the influenza-like illness is the occurrence of fever greater than or equal to 38° C. for at least 24 hours and at least one of cough, sputum production, wheezing, difficulty breathing, nasal congestion, rhinorrhea, pharyngitis, otitis, vomiting, diarrhea, sore throat, chills (shivering), tiredness (fatigue), headache, and myalgia (muscle aches).

In some embodiments, administration of the RIPK1 inhibitor reduces CRP level by at least 50% within about 3 days of treatment.

In some embodiments, administration of the RIPK1 Inhibitor reduces plasma levels of one or more cytokines selected from IL-4, IL-6, IL-10, IL-17, TNFα, or IFNγ in a subject. In some embodiments, administration of the RIPK1 inhibitor reduces plasma levels of one or more cytokines selected from IL-10, IL-6, IFNγ, or chemokine (C-X-C motif) Ligand 10. In some embodiments, administration of the RIPK1 Inhibitor reduces plasma levels of IL-10. In some embodiments, administration of the RIPK1 Inhibitor reduces plasma levels of IL-6. In some embodiments, administration of the RIPK1 Inhibitor reduces plasma levels of IL-8. In some embodiments, administration of the RIPK1 Inhibitor reduces plasma levels of IFNγ.

In some embodiments, administration of the RIPK1 inhibitor reduces the number of leukocytes or the neutrophil-to-lymphocyte ratio. In some embodiments, administration of the RIPK1 inhibitor reduces the number of leukocytes or the neutrophil-to-lymphocyte ratio within 7 days of the treatment. In some embodiments, administration of the RIPK1 inhibitor reduces the number of leukocytes. In some embodiments, administration of the RIPK1 inhibitor reduces the neutrophil-to-lymphocyte ratio.

In some embodiments, administration of the RIPK1 inhibitor increases saturation oxygen (SPO₂) level. In some embodiments, administration of the RIPK1 inhibitor increases 50% saturation oxygen (SPO₂) recovery rate within 7 days of treatment. In some embodiments, administration of the RIPK1 inhibitor increases SPO₂/FiO₂ ratio. In some embodiments, administration of the RIPK1 inhibitor increases SPO₂/FiO₂ ratio after 7 days of the treatment.

In some embodiments, administration of the RIPK1 inhibitor reduces and/or eliminates the need for oxygen support. In some embodiments, administration of the RIPK1 inhibitor reduces and/or eliminates the need of a ventilator. In some embodiments, administration of the RIPK1 inhibitor reduces and/or eliminates respiratory failure.

In some embodiments, the RIPK1 Inhibitor is administered as monotherapy. In some embodiments, one or more active compounds are administered with the RIPK1 Inhibitor. In some embodiments, one or more active compounds is selected from analgesics, decongestants, expectorants, antihistamines, mucokinetics, and cough suppressants. The additional therapeutic agent(s) may be administered concurrently or sequentially with the RIPK1 Inhibitor.

In some embodiments, one or more antiviral therapies are administered with the RIPK1 Inhibitor. The administration may be prior to the compound administration, concurrently with the compound administration, or following the compound administration. In some embodiments, one or more antiviral therapies may be administered by using one or more antiviral agents. In some embodiments the antiviral agents are selected from remdesivir, hydroxychloroquinine, galidesivir, oseltamivir, paramivir, zanamivir, ganciclovir, acyclovir, ribavirin, lopinavir, ritonavir, favipiravir, darunavir or a combination thereof.

In some embodiments, the subject was previously administered an antiviral therapy by administering one or more antiviral agents. In some embodiments, the antiviral agents are selected from remdesivir, hydroxychloroquinine, galidesivir, oseltamivir, paramivir, zanamivir, ganciclovir, acyclovir, ribavirin, lopinavir, ritonavir, favipiravir, darunavir or a combination thereof.

In some embodiments, one or more steroids, such as corticosteroids, are administered with the RIPK Inhibitor. Exemplary corticosteroids include, but are not limited to, dexamethasone, betamethasone, prednisone, prednisolone, methylprednisolone, cortisone, hydrocortisone, triamcinolone, or ethamethasone, or a pharmaceutically acceptable salt thereof. In some embodiments, the corticosteroid is dexamethasone. The administration may be prior to the compound administration, concurrently with the compound administration, or following the compound administration. The corticosteroid used in the disclosed methods may be administered according to regimens known in the art, e.g., US FDA-approved regimens.

In some embodiments, the subject was previously administered one or more steroids, such as corticosteroids. In some embodiments, the one or more corticosteroids are selected from dexamethasone, betamethasone, prednisone, prednisolone, methylprednisolone, cortisone, hydrocortisone, triamcinolone, or ethamethasoneb, or a pharmaceutically acceptable salt thereof.

In some embodiments, the subject has high IL-6 levels and/or high CRP levels.

This disclosure further provides a method of determining if a subject with infectious disease characterized by CRS has an increased propensity for effective treatment of CRS or reducing one or more symptoms associated with CRS comprising measuring a concentration of CRP in a serum sample from the subject wherein if the serum sample has a concentration of CRP greater than the upper limit of normal, the subject has an increased propensity for effective treatment of CRS or reducing one or more symptoms associated with CRS.

In another aspect, the disclosure provides a method of determining if a subject with infectious disease characterized by CRS has an increased propensity for effective treatment of CRS or reducing one or more symptoms associated with CRS comprising measuring a concentration of IL-6 in a serum sample from the subject wherein if the serum sample has a concentration of IL-6 greater than the upper limit of normal, the subject has an increased propensity for effective treatment of CRS or reducing one or more symptoms associated with CRS.

### III. Therapeutic Methods

Provided herein are methods of treating a subject at risk of or having CRS comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of treating a subject at risk of or having SIRS comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of treating a subject in a hyperinflammatory state comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of reducing inflammation in a subject at risk of or having CRS comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of reducing inflammation in a subject at risk of or having SIRS comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of reducing organ damage in a subject in a hyperinflammatory state, including in a subject at risk of or having CRS, comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of reducing organ damage in a subject in a hyperinflammatory state, including in a subject at risk of or having SIRS comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of reducing sepsis-related inflammation or organ injury in a subject comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of treating a subject having influenza-like illness comprising administering to a subject in need thereof a therapeutically effective amount of a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

Provided herein are methods of reducing symptoms related to coronavirus infection comprising administering to a subject in need thereof a therapeutically effective amount of the RIPK1 Inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

In some embodiments the therapeutically effective amount is about 5 to about 1000 mg. In some embodiments the therapeutically effective amount is about 400 mg to about 1000 mg. In some embodiments, the subject is a mammal. In some embodiments, the mammal is a human.

In some embodiments, a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg is administered. In some embodiments, the dose is 5 mg, 10 mg, 15 mg, 20 mg, 25 mg, 30 mg, 35 mg, 40 mg, 45 mg, 50 mg, 55 mg, 60 mg, 100 mg, 200 mg, 300 mg, 400 mg, 600 mg, 800 mg, or 1000 mg. In some embodiments, the dose is 5 mg. In some embodiments, the dose is 15 mg. In some embodiments, a dose of about 400 mg to about 1000 mg is administered. In some embodiments, the dose is 400 mg. In some embodiments, the dose is 600 mg. In some embodiments, the dose is 800 mg. In some embodiments, the dose is 1000 mg.

In some embodiments, the dose is administered daily. The daily dose can be delivered as a single dose or split into multiple parts. For example, in some embodiments, the dose is administered once a day (e.g., about every 24 hours). In some embodiments, the dose is administered twice daily. In some embodiments, the dose is subdivided in two parts to be administered twice per day (e.g., about every 12 hours). In some embodiments, the dose is subdivided in three parts to be administered three times per day (e.g., about every 8 hours). In some embodiments, the dose is subdivided in four parts to be administered four times per day (e.g., about every 6 hours).

In some embodiments, the dose is administered orally. In some embodiments, the dose is administered in the form of tablets. In some embodiments, the dose is administered in the form of pills, capsules, semisolids, powders, sustained release formulations, solutions, suspensions, elixirs, aerosols, or any other appropriate compositions. In cases where the subject is unable to ingest the dose orally, a gastric feeding tube, a nasal feeding tube, or I.V. may be used. In some embodiments, the dose is administered orally. In some embodiments, the dose is administered via a gastric feeding tube.

Determination of the frequency of administration can be made by persons skilled in the art, such as an attending physician based on considerations of the condition being treated, age of the subject being treated, severity of the condition being treated, general state of health of the subject being treated and the like. In some embodiments, the RIPK1 Inhibitor is administered in a therapeutically effective amount for treatment of SARS-CoV-2 infection. The therapeutically effective amount is typically dependent on the weight of the subject being treated, his or her physical or health condition, the extensiveness of the condition to be treated, or the age of the subject being treated, pharmaceutical formulation methods, and/or administration methods (e.g., administration time and administration route).

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability. Bioavailability of drugs that decompose at stomach pH can be increased by administration of such drugs in a formulation that releases the drug intraduodenally.

The compositions are comprised of in general, the RIPK1 Inhibitor and/or a pharmaceutically acceptable salt thereof in combination with a pharmaceutically acceptable excipient such as binders, surfactants, diluents, buffering agents, antiadherents, glidants, hydrophilic or hydrophobic polymers, retardants, stabilizing agents or stabilizers, disintegrants or superdisintegrants, antioxidants, antifoaming agents, fillers, flavors, colors, lubricants, sorbents, preservatives, plasticizers, or sweeteners, or mixtures thereof, which facilitate processing of the RIPK1 Inhibitor and/or a pharmaceutically acceptable salt thereof into preparations which can be used pharmaceutically. Any of the well-known techniques and excipients may be used as suitable and as understood in the art, see for example, Remington: The Science and Practice of Pharmacy, Twenty-first Ed., (Pharmaceutical Press, 2005); Liberman, H. A., Lachman, L., and Schwartz, J. B. Eds., Pharmaceutical Dosage Forms, Vol. 1-2 Taylor & Francis 1990; and R. I. Mahato, Ansel's Pharmaceutical Dosage Forms and Drug Delivery Systems, Second Ed. (Taylor & Francis, 2012).

In certain embodiments, the formulations may include one or more pH adjusting agents or buffering agents, for example, acids such as acetic, boric, citric, fumaric, maleic, tartaric, malic, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate, ammonium chloride, and the like. Such buffers used as bases may have other counterions than sodium, for example, potassium, magnesium, calcium, ammonium, or other counterions. Such acids, bases and buffers are included in an amount required to maintain pH of the composition in an acceptable range.

In certain embodiments, the formulations may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In certain embodiments, the formulations may also include one or more antifoaming agents to reduce foaming during processing which can result in coagulation of aqueous dispersions, bubbles in the finished film, or generally impair processing. Exemplary anti-foaming agents include silicon emulsions or sorbitan sesquoleate.

In certain embodiments, the formulations may also include one or more antioxidants, such as non-thiol antioxidants, for example, butylated hydroxytoluene (BHT), sodium ascorbate, ascorbic acid or its derivative, and tocopherol or its derivatives. In certain embodiments, antioxidants enhance chemical stability where required. Other agents such as citric acid or citrate salts or EDTA may also be added to slow oxidation.

In certain embodiments, the formulations may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

In certain embodiments, the formulations may also include one or more binders. Binders impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinyl-pyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum mucilage of isapol husks, polyvinylpyrrolidone (e.g., Polyvidone® CL, Kollidon® CL, Polyplasdone® XL-10), larch arabogalactan, Veegum®, polyethylene glycol, polyethylene oxide, waxes, sodium alginate, and the like.

In certain embodiments, the formulations may also include dispersing agents and/or viscosity modulating agents. Dispersing agents and/or viscosity modulating agents include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween®60 or 80, PEG, polyvinylpyrrolidone (PVP; commercially known as Plasdone®), and the carbohydrate-based dispersing agents such as, for example, hydroxypropyl celluloses (e.g., HPC, H-PC-SL, and HPC-L), hydroxypropyl methylcelluloses (e.g., HPMC K100, RPMC K4M, HPMC K15M, and HPMC K100M), carboxymethylcellulose sodium, methylcellulose, hydroxyethyl-cellulose, hydroxypropyl-cellulose, hydroxypropylmethylcellulose phthalate, hydroxypropyl-methylcellulose acetate stearate (HPMCAS), noncrystalline cellulose, polyethylene oxides, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F10®8, which are block copolymers of ethylene oxide and propylene oxide); and poloxamines (e.g., Tetronic 908®, also known as Poloxamine 908®, which is a tetrafonctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to 5400, sodium carboxymethylcellulose, methylcellulose, polysorbate-80, sodium alginate, gums, e.g., gum tragacanth and gum acacia, guar gum, xanthans, including xanthan gum, sugars, cellulosics, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate. In general, binder levels of about 10 to about 70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 90% and more typically up to 70% in tablet formulations is common.

In certain embodiments, the formulations may also include one or more diluents which refer to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); hydroxypropyl-methylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the formulations may also include one or more disintegrants which includes both the dissolution and dispersion of the dosage form when contacted with gastrointestinal fluid. Disintegration agents or disintegrants facilitate the breakup or disintegration of a substance. Examples of disintegration agents include a starch, e.g., a natural starch like corn starch or potato starch, a pregelatinized starch like National 1551 or sodium starch glycolate such as Promogel® or Explotab®, a cellulose like a wood product, methylcrystalline cellulose, e.g., Avicel®, Avicel® PH101, Avicel® PH 102, Avicel® PH105, Elceme® P100, Emcocel®, Vivacel®, and Solka-Floc®, methylcellulose, croscarmellose, or a cross-linked cellulose like cross-linked sodium carboxymethyl-cellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In certain embodiments, the formulations may also include erosion facilitators. Erosion facilitators include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

In certain embodiments, the formulations may also include one or more filling agents which include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In certain embodiments, the formulations may also include one or more flavoring agents and/or sweeteners e.g., acacia syrup, acesulfame K, alitame, anise, apple, aspartame, banana, Bavarian cream berry, black currant, butterscotch, calcium citrate, camphor, caramel, cherry, cherry cream chocolate, cinnamon, bubble gum, citrus, citrus punch, citrus cream, cotton candy, cocoa, cola, cool cherry, cool citrus, cyclamate, cyclamate, dextrose, eucalyptus, eugenol, fructose, fruit punch, ginger, glycyrrhizinate, glycyrrhiza (licorice) syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, powder, raspberry, root beer, rum, saccharin, safrole, sorbitol, spearmint, spearmint cream, strawberry, strawberry cream, Stevia, sucralose, sucrose, sodium saccharin, saccharin, aspartame, acesulfame potassium, mannitol, talin, xylitol, sucralose, sorbitol, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

In certain embodiments, the formulations may also include one or more lubricants and glidants which are compounds that prevent, reduce or inhibit adhesion or friction of materials. Exemplary lubricants include stearic acid, calcium hydroxide, talc, sodium stearyl lumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG4000) or a methoxypolyethylene glycol such as Carbowax®, sodium oleate, sodium benzoate, glyceryl behenate, polyethylene glycol, magnesium or sodium lauryl sulfate, colloidal silica such as Syloid®, Cab-O-Sil®, a starch such as corn starch, silicone oil, a surfactant, and the like.

In certain embodiments, the formulations may also include one or more plasticizers which are compounds used to soften the enteric or delayed release coatings to make them less brittle. Suitable plasticizers include polyethylene glycols such as PEG 300, PEG 400, PEG 600, PEG 1450, PEG 3350, and PEG 800, stearic acid, propylene glycol, oleic acid, triethyl citrate, dibutyl sebacate, triethyl cellulose and triacetin. In some embodiments, plasticizers can also function as dispersing agents or wetting agents.

In certain embodiments, the formulations may also include one or more solubilizers which include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins for example Captisol®, ethanol, n-butanol, isopropyl alcohol, cholesterol, bile salts, polyethylene glycol 200-600, glycofurol, transcutol, propylene glycol, and dimethyl isosorbide and the like. In one embodiment, the solubilizer is vitamin E TPGS and/or Captisol® or β-hydroxypropylcyclodextrin.

In certain embodiments, the formulations may also include one or more suspending agents which include compounds such as polyvinylpyrrolidone, e.g., polyvinylpyrrolidone K112, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, e.g., gum tragacanth and gum acacia, guar gum, xanthans, including xanthan gum, sugars, cellulosics, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monoleate, povidone and the like.

In certain embodiments, the formulations may also include one or more surfactants which include compounds such as sodium lauryl sulfate, sodium docusate, Tween 20, 60 or 80, triacetin, vitamin E TPGS, sorbitan monooleate, polyoxyethylene sorbitan monooleate, polyoxyethylene sorbitan monolaurate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g. octoxynol 10, octoxynol 40. In some embodiments, surfactants may be included to enhance physical stability or for other purposes.

In certain embodiments, the formulations may also include one or more viscosity enhancing agents which include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol alginates, acacia, chitosans and combinations thereof.

In certain embodiments, the formulations may also include one or more wetting agents which include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Pharmaceutical preparations disclosed herein can be obtained by mixing one or more solid excipient such as carrier, binder, filling agent, suspending agent, flavoring agent, sweetening agent, disintegrating agent, dispersing agent, surfactant, lubricant, colorant diluent, solubilizer, moistening agent, plasticizer, stabilizer, penetration enhancer, wetting agent, anti-foaming agent, antioxidant, preservative, or one or more combination thereof with one or more of the compounds described herein, optionally grinding the resulting mixture, and processing the mixture of granules, after adding suitable excipients, if desired, to obtain tablets.

Pharmaceutical preparations disclosed herein also include capsules made of gelatin, as well as soft, sealed capsules made of gelatin and a plasticizer, such as glycerol or sorbitol. Capsules may also be made of polymers such as hypromellose. The capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, lipids, solubilizers, or liquid polyethylene glycols. In addition, stabilizers may be added. All formulations for oral administration should be in dosages suitable for such administration.

These formulations can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, (6) fusion, or (7) extrusion. See, e.g., Lachman et al., The Theory and Practice of Industrial Pharmacy, 3^{rd} ed. (1986). Other methods include, e.g., spray drying, pan coating, melt granulation, granulation, fluidized bed spray drying or coating (e.g., wurster coating), tangential coating, top spraying, tableting, extruding, extrusion/spheronization, and the like.

It should be appreciated that there is considerable overlap between excipients used in the solid dosage forms described herein. Thus, the above-listed additives should be taken as merely exemplary, and not limiting, of the types of excipient that can be included in solid dosage forms described herein. The type and amounts of such excipient can be readily determined by one skilled in the art, according to the particular properties desired.

In some embodiments, the solid dosage forms described herein are enteric coated oral dosage forms, i.e., as an oral dosage form of a pharmaceutical composition as described herein which utilizes an enteric coating to effect the release of the compound in the intestine of the gastrointestinal tract. An “enterically coated” drug and/or tablet refers to a drug and/or tablet that is coated with a substance that remains intact in the stomach but dissolves and releases the drug once the intestine (in one embodiment small intestine) is reached. As used herein “enteric coating”, is a material, such as a polymer material or materials which encase the therapeutically active agent core either as a dosage form or as particles. Typically, a substantial amount or all of the enteric coating material is dissolved before the therapeutically active agent is released from the dosage form, so as to achieve delayed dissolution of the therapeutically active agent core or particles in the small and/or large intestine. Enteric coatings are discussed, for example, Loyd, V. Allen, Remington: The Science and Practice of Pharmacy, Twenty-first Ed., (Pharmaceutical Press, 2005; and P. J. Tarcha, Polymers for Controlled Drug Delivery, Chapter 3, CRC Press, 1991. Methods for applying enteric coatings to pharmaceutical compositions are well known in the art, and include for example, U.S. Patent Publication No. 2006/0045822.

The enteric coated dosage form may be a compressed or molded or extruded tablet (coated or uncoated) containing granules, powder, pellets, beads or particles of the RIPK1 Inhibitor and/or a pharmaceutically acceptable salt thereof and/or other excipients, which are themselves coated or uncoated provided at least the tablet or the RIPK1 Inhibitor is coated. The enteric coated oral dosage form may also be a capsule (coated or uncoated) containing pellets, beads or granules of the RIPK1 Inhibitor and/or a pharmaceutically acceptable salt thereof and/or other excipients, which are themselves coated or uncoated provided at least one of them is coated. Some examples of coatings that were originally used as enteric coatings are beeswax and glyceryl monostearate; beeswax, shellac and cellulose; and cetyl alcohol, mastic and shellac as well as shellac and stearic acid (U.S. Pat. No. 2,809,918); polyvinylacetate and ethyl cellulose (U.S. Pat. No. 3,835,221). More recently, the coatings used are neutral copolymers of polymethacrylic acid esters (Eudragit L30D). (F. W. Goodhart et al, Pharm. Tech., p. 64-71, April, 1984); copolymers of methacrylic acid and methacrylic acid methyl ester (Eudragit S), or a neutral copolymer of polymethacrylic acid esters containing metallic stearates (Mehta et al U.S. Pat. Nos. 4,728,512 and 4,794,001), cellulose acetate succinate, and hypromellose phthalate.

Any anionic polymer exhibiting a pH-dependent solubility profile can be used as an enteric coating in the methods and compositions described herein to achieve delivery to the intestine. In one embodiment, delivery can be to the small intestine. In another embodiment, delivery can be to the duodenum. In some embodiments the polymers described herein are anionic carboxylic polymers. In other embodiments, the polymers and compatible mixtures thereof, and some of their properties, include, but are not limited to:

Shellac: Also called purified lac, it is a refined product obtained from the resinous secretion of an insect. This coating dissolves in media of pH>7;

Acrylic polymers: The performance of acrylic polymers (primarily their solubility in biological fluids) can vary based on the degree and type of substitution. Examples of suitable acrylic polymers include methacrylic acid copolymers and ammonium methacrylate copolymers. The Eudragit series L, S, and RS (manufactured Rohm Pharma and known as Evonik®) are available as solubilized in organic solvent, aqueous dispersion, or dry powders. The Eudragit series RL, NE, and RS are insoluble in the gastrointestinal tract but are permeable and are used primarily for colonic targeting. The Eudragit series L, L-30D and S are insoluble in stomach and dissolve in the intestine and may be selected and formulated to dissolve at a value of pH greater than 5.5 or as low as greater than 5 or as high as greater than 7;

Cellulose Derivatives: Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP pseudolatex with particles<1 μm. Other components in Aquateric can include pluronics, Tweens, and acetylated monoglycerides. Other suitable cellulose derivatives include: cellulose acetate tritnellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (HPMCAS e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP): PVAP dissolves in pH>5, and it is much less permeable to water vapor and gastric fluids. Detailed description of above polymers and their pH-dependent solubility can be found at in the article titled “Enteric coated hard gelatin capsules” by Professor Karl Thoma and Karoline Bechtold at http://pop.www.capsugel.com/media/library/enteric-coated-hard-gelatin-capsules.pdf. In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually contain 10-25% by weight of a plasticizer, especially dibutyl phthalate, polyethylene glycol, triethyl citrate and triacetin. Conventional coating techniques such as fluid bed or Wurster coaters, or spray or pan coating are employed to apply coatings. The coating thickness must be sufficient to ensure that the oral dosage form remains intact until the desired site of topical delivery in the intestinal tract is reached.

Colorants, surfactants, anti-adhesion agents, antifoaming agents, lubricants (e.g., carnauba wax or PEG) and other additives may be added to the coatings besides plasticizers to solubilize or disperse the coating material, and to improve coating performance and the coated product.

To accelerate the dissolution of the enteric coat, a half-thickness, double coat of enteric polymer (for instance, Eudragit L30 D-55) may be applied, and the inner enteric coat may have a buffer up to pH 6.0 in the presence of 10% citric acid, followed by a final layer of standard Eudragit L 30 D-55. Applying two layers of enteric coat, each half the thickness of a typical enteric coat, Liu and Basit were able to accelerate enteric coating dissolution compared to a similar coating system applied, unbuffered, as a single layer (Liu, F. and Basit, A. Journal of Controlled Release. 147 (2010) 242-245.)

The intactness of the enteric coating may be measured, for example, by the degradation of the drug within the micropellets. The enteric coated dosage forms or pellets may be tested in dissolution testing first in gastric fluid and separately in intestinal fluid as described in USP to determine its function.

The enteric coated tablets and capsules formulation containing the disclosed compounds can be made by methods well known in the art. For example, tablets containing a compound disclosed herein can be enterically coated with a coating solution containing Eudragit®, diethylphthlate, isopropyl alcohol, talc, and water using a side vented coating pan (Freund Hi-Coater).

Alternatively, a multi-unit dosage form comprising enteric-coated pellets that can be incorporated into a tablet or into a capsule can be prepared as follows.

Core material: The core material for the individually enteric coating layered pellets can be constituted according to different principles. Seeds layered with the active agent (i.e., the RIPK1 Inhibitor and/or a pharmaceutically acceptable sale thereof), optionally mixed with alkaline substances or buffer, can be used as the core material for the further processing. The seeds which are to be layered with the active agent can be water insoluble seeds comprising different oxides, celluloses, organic polymers and other materials, alone or in mixtures or water-soluble seeds comprising different inorganic salts, sugars, non-pareils and other materials, alone or in mixtures. Further, the seeds may comprise the active agent in the form of crystals, agglomerates, compacts etc. The size of the seeds is not essential for the present disclosure but may vary between approximately 0.1 and 2 mm. The seeds layered with the active agent are produced either by powder or solution/suspension layering using for instance granulation or spray coating layering equipment.

Before the seeds are layered, active agent may be mixed with further components. Such components can be binders, surfactants, fillers, disintegrating agents, alkaline additives or other and/or pharmaceutically acceptable ingredients alone or in mixtures. The binders are for example polymers such as hydroxypropyl methylcellulose (HPMC), hydroxypropyl-cellulose (HPC), carboxymethylcellulose sodium, polyvinyl pyrrolidone (PVP), or sugars, starches or other pharmaceutically acceptable substances with cohesive properties. Suitable surfactants are found in the groups of pharmaceutically acceptable non-ionic or ionic surfactants such as for instance sodium lauryl sulfate.

Alternatively, the active agent optionally mixed with suitable constituents can be formulated into a core material. Said core material may be produced by extrusion/spheronization, balling or compression utilizing conventional process equipment. The size of the formulated core material is approximately between 0.1 and 4 mm and for example, between 0.1 and 2 mm. The manufactured core material can further be layered with additional ingredients comprising the active agent and/or be used for further processing.

The active agent is mixed with pharmaceutical constituents to obtain preferred handling and processing properties and a suitable concentration of the active agent in the final preparation. Pharmaceutical constituents such as fillers, binders, lubricants, disintegrating agents, surfactants and other pharmaceutically acceptable additives may be used.

Alternatively, the aforementioned core material can be prepared by using spray drying or spray congealing technique.

Enteric Coating Layer(s): Before applying the enteric coating layer(s) onto the core material in the form of individual pellets, the pellets may optionally be covered with one or more separating layer(s) comprising pharmaceutical excipients optionally including alkaline compounds such as pH-buffering compounds. This/these separating layer(s), separate(s) the core material from the outer layers being enteric coating layer(s). This/these separating layer(s) protecting the core material of active agent should be water soluble or rapidly disintegrating in water.

A separating layer(s) can be optionally applied to the core material by coating or layering procedures in suitable equipment such as coating pan, coating granulator or in a fluidized bed apparatus using water and/or organic solvents for the coating process. As an alternative the separating layer(s) can be applied to the core material by using powder coating technique. The materials for the separating layers are pharmaceutically acceptable compounds such as, for instance, sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium, water soluble salts of enteric coating polymers and others, used alone or in mixtures. Additives such as plasticizers, colorants, pigments, fillers anti-tacking and anti-static agents, such as for instance magnesium stearate, titanium dioxide, talc and other additives may also be included into the separating layer(s).

When the optional separating layer is applied to the core material it may constitute a variable thickness. The maximum thickness of the separating layer(s) is normally only limited by processing conditions. The separating layer may serve as a diffusion barrier and may act as a pH-buffering zone. The optionally applied separating layer(s) is not essential for the embodiments of the present disclosure. However, the separating layer(s) may improve the chemical stability of the active substance and/or the physical properties of the novel multiple unit tableted dosage form.

Alternatively, the separating layer may be formed in situ by a reaction between an enteric coating polymer layer applied on the core material and an alkaline reacting compound in the core material. Thus, the separating layer formed comprises a water-soluble salt formed between the enteric coating layer polymer(s) and an alkaline reacting compound which is in the position to form a salt.

One or more enteric coating layers are applied onto the core material or onto the core material covered with separating layer(s) by using a suitable coating technique. The enteric coating layer material may be dispersed or dissolved in either water or in suitable organic solvents. As enteric coating layer polymers, one or more, separately or in combination, of the following can be used, e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating polymer(s).

The enteric coating layers contain pharmaceutically acceptable plasticizers to obtain the desired mechanical properties, such as flexibility and hardness of the enteric coating layers. Such plasticizers are for instance, but not restricted to triacetin, citric acid esters, phthalic acid esters, dibutyl sebacate, cetyl alcohol, polyethylene glycols, polysorbates or other plasticizers.

The amount of plasticizer is optimized for each enteric coating layer formula, in relation to the selected enteric coating layer polymer(s), selected plasticizer(s) and the applied amount of said polymer(s), in such a way that the mechanical properties, i.e. flexibility and hardness of the enteric coating layer(s), for instance exemplified as Vickers hardness, are adjusted so that if a tablet is desired the acid resistance of the pellets covered with enteric coating layer(s) does not decrease significantly during compression of pellets into tablets. The amount of plasticizer is usually above 5% by weight of the enteric coating layer polymer(s), such as 15-50% and further such as 20-50%. Additives such as dispersants, colorants, pigments polymers e.g. poly(ethylacrylate, methylmethacrylate), anti-tacking and anti-foaming agents may also be included into the enteric coating layer(s). Other compounds may be added to increase film thickness and to decrease diffusion of acidic gastric juices into the acid susceptible material. The maximum thickness of the applied enteric coating is normally only limited by processing conditions and the desired dissolution profile.

Over-Coating Layer: Pellets covered with enteric coating layer(s) may optionally further be covered with one or more over-coating layer(s). The over-coating layer(s) should be water soluble or rapidly disintegrating in water. The over-coating layer(s) can be applied to the enteric coating layered pellets by coating or layering procedures in suitable equipment such as coating pan, coating granulator or in a fluidized bed apparatus using water and/or organic solvents for the coating or layering process. The materials for over-coating layers are chosen among pharmaceutically acceptable compounds such as sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium and others, used alone or in mixtures. Additives such as plasticizers, colorants, pigments, fillers, anti-tacking and anti-static agents, such for instance magnesium stearate, titanium dioxide, talc and other additives may also be included into the over-coating layer(s). The over-coating layer may further prevent potential agglomeration of enteric coating layered pellets, further it may protect the enteric coating layer towards cracking during the compaction process and enhance the tableting process. The maximum thickness of the applied over-coating layer(s) is normally limited by processing conditions and the desired dissolution profile. The over-coating layer may also be used as a tablet film coating layer.

Enteric coating of soft gelatin capsules may contain an emulsion, oil, microemulsion, self-emulsifying system, lipid, triglycerides, polyethylene glycol, surfactants, other solubilizers and the like, and combinations thereof, to solubilize the active agent. The flexibility of the soft gelatin capsule is maintained by residual water and plasticizer. Moreover, for gelatin capsules the gelatin may be dissolved in water so that spraying must be accomplished at a rate with relatively low relative humidity such as can be accomplished in a fluid bed or Wurster. In addition, drying should be accomplished without removing the residual water or plasticizer causing cracking of the capsule shell. Commercially available blends optimized for enteric coating of soft gelatin capsules such as Instamodel EPD (Enteric Polymeric Dispersion), available from Ideal Cures, Pvt. Ltd. (Mumbai, India). On a laboratory scale enteric coated capsules may be prepared by: a) rotating capsules in a flask or dipping capsules in a solution of the gently heated enteric coating material with plasticizer at the lowest possible temperature or b) in a lab scale sprayer/fluid bed and then drying.

For aqueous active agents, it can be especially desirable to incorporate the drug in the water phase of an emulsion. Such “water-in-oil” emulsion provides a suitable biophysical environment for the drug and can provide an oil-water interface that can protect the drug from adverse effects of pH or enzymes that can degrade the drug. Additionally, such water-in-oil formulations can provide a lipid layer, which can interact favorably with lipids in cells of the body, and can increase the partition of the formulation onto the membranes of cells. Such partition can increase the absorption of drugs in such formulations into the circulation and therefore can increase the bioavailability of the drug.

In some embodiments the water-in-oil emulsion contains an oily phase composed of medium or long chain carboxylic acids or esters or alcohols thereof, a surfactant or a surface-active agent, and an aqueous phase containing primarily water and the active agent.

Medium and long chain carboxylic acids are those ranging from C₈ to C₂₂ with up to three unsaturated bonds (also branching). Examples of saturated straight chain acids are n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. Also useful are unsaturated monoolefinic straight chain monocarboxylic acids. Examples of these are oleic acid, gadoleic acid and erucic acid. Also useful are unsaturated (polyolefinic) straight chain monocarboxylic acids. Examples of these are linoleic acid, ricinoleic acid, linolenic acid, arachidonic acid and behenolic acid. Useful branched acids include, for example, diacetyl tartaric acid. Unsaturated olefinic chains may also be hydroxylated or ethoxylated to prevent oxidation or to alter the surface properties.

Examples of long chain carboxylic acid esters include, but are not limited to, those from the group of: glyceryl monostearates; glyceryl monopalmitates; mixtures of glyceryl monostearate and glyceryl monopalmitate; glyceryl monolinoleate; glyceryl monooleate; mixtures of glyceryl monopalmitate, glyceryl monostearate, glyceryl monooleate and glyceryl monolinoleate; glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate; acetylated glycerides such as distilled acetylated monoglycerides; mixtures of propylene glycol monoesters, distilled monoglycerides, sodium steroyl lactylate and silicon dioxide; d-alpha tocopherol polyethylene glycol 1000 succinate; mixtures of mono- and di-glyceride esters such as Atmul; calcium stearoyl lactylate; ethoxylated mono- and di-glycerides; lactated mono- and di-glycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; stearyl octanoate; C₈-C₃₀ cholesterol/lavosterol esters; and sucrose long chain carboxylic acid esters. Examples of the self-emulsifying long chain carboxylic acid esters include those from the groups of stearates, palmitates, ricinoleates, oleates, behenates, ricinolenates, myristates, laurates, caprylates, and caproates. In some embodiments the oily phase may comprise a combination of 2 or more of the long chain carboxylic acids or esters or alcohols thereof. In some embodiments medium chain surfactants may be used and the oil phase may comprise a mixture of caprylic/capric triglyceride and C₈/C₁₀ mono-/di-glycerides of caprylic acid, glyceryl caprylate or propylene glycol monocaprylate or their mixtures.

The alcohols that can be used are exemplified by the hydroxyl forms of the carboxylic acids exemplified above and also stearyl alcohol.

Surface active agents or surfactants are long chain molecules that can accumulate at hydrophilic/hydrophobic (water/oil) interfaces and lower the surface tension at the interface. As a result, they can stabilize an emulsion. In some embodiments, the surfactant may comprise: Tween® (polyoxyethylene sorbate) family of surfactants, Span® (sorbitan long chain carboxylic acid esters) family of surfactants, Pluronic® (ethylene or propylene oxide block copolymers) family of surfactants, Labrasol®, Labrafil® and Labrafac® (each polyglycolyzed glycerides) families of surfactants, sorbitan esters of oleate, stearate, laurate or other long chain carboxylic acids, poloxamers (polyethylene-polypropylene glycol block copolymers or Pluronic®.), other sorbitan or sucrose long chain carboxylic acid esters, mono and diglycerides, PEG derivatives of caprylic/capric triglycerides and mixtures thereof or mixture of two or more of the above. In some embodiments the surfactant phase may comprise a mixture of polyoxyethylene (20) sorbitan monooleate (Tween 80®) and sorbitan monooleate (Span 80®).

The aqueous phase may optionally comprise the active agent suspended in water and a buffer.

In some embodiments, such emulsions are coarse emulsions, microemulsions and liquid crystal emulsions. In other embodiments such emulsion may optionally comprise a permeation enhancer. In other embodiments, spray-dried dispersions or microparticles or nanoparticles containing encapsulated microemulsion, coarse emulsion or liquid crystal can be used.

In some embodiments, the solid dosage forms described herein are non-enteric time-delayed release dosage forms. The term “non-enteric time-delayed release” as used herein refers to the delivery so that the release of the drug can be accomplished at some generally predictable location in the intestinal tract more distal to that which would have been accomplished if there had been no delayed release alterations. In some embodiments the method for delay of release is a coating that becomes permeable, dissolves, ruptures, and/or is no longer intact after a designed duration. The coating in the time-delayed release dosage forms can have a fixed time to erode after which the drug is released (suitable coating include polymeric coating such as HPMC, PEO, and the like) or has a core comprised of a superdisintegrant(s) or osmotic agent(s) or water attractant such as a salt, hydrophilic polymer, typically polyethylene oxide or an alkylcellulose, salts such as sodium chloride, magnesium chloride, sodium acetate, sodium citrate, sugar, such as glucose, lactose, or sucrose, or the like, which draw water through a semi-permeable membrane or a gas generating agent such as citric acid and sodium bicarbonate with or without an acid such as citric acid or any of the aforementioned acids incorporated in dosage forms. The semi-permeable membrane, while mostly not permeable to the drug nor the osmotic agent, is permeable to water that permeates at a near constant rate to enter the dosage form to increase the pressure and ruptures after the swelling pressure exceeds a certain threshold over a desired delay time. The permeability through this membrane of the drug should be less than 1/10 than water and in one embodiment less than 1/100 the water permeability. Alternatively, a membrane could become porous by leaching an aqueous extractable over a desired delay time.

Osmotic dosage forms have been described in Theeuwes U.S. Pat. No. 3,760,984, and an osmotic bursting dosage form is described in Baker U.S. Pat. No. 3,952,741. This osmotic bursting dosage form can provide a single pulse of release or multiple pulses if different devices with different timings are employed. The timing of the osmotic burst may be controlled by the choice of polymer and the thickness or the area of the semipermeable membrane surrounding the core that contains both the drug and the osmotic agent or attractant. As the pressure in the dosage form increase with additional permeated water, the membrane elongates until its breaking point, and then the drug is released. Alternatively, specific areas of rupture can be created in the membrane by having a thinner, weaker area in the membrane or by adding a weaker material to an area of the coating membrane. Some preferred polymers with high water permeabilities that may be used as semipermeable membranes are cellulose acetate, cellulose acetate butyrate, cellulose nitrate, crosslinked polyvinyl, alcohol, polyurethanes, nylon 6, nylon 6.6, and aromatic nylon. Cellulose acetate is an especially preferred polymer.

In another embodiment, the time-delayed coating that begins its delay to releasing drug after the enteric coating is at least partially dissolved is comprised of hydrophilic, erodible polymers that upon contact with water begin to gradually erode over time. Examples of such polymers include cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; polyalkylene oxides, such as polyethylene oxide or polyethylene glycols, particularly high molecular weight polyethylene glycols; chitosan; poly(vinyl alcohol); xanthan gum; maleic anhydride copolymers; poly(vinyl pyrrolidone); starch and starch-based polymers; maltodextrins; poly (2-ethyl-2-oxazoline); poly(ethyleneimine); polyurethane; hydrogels; crosslinked polyacrylic acids; and combinations or blends of any of the foregoing.

Some preferred erodible hydrophilic polymers suitable for forming the erodible coating are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 10⁵ Daltons to about 10⁷ Daltons. A preferred molecular weight range of poly(ethylene oxide) polymers is from about 2×10⁵ to 2×10⁶ Daltons and is commercially available from The Dow Chemical Company (Midland, Mich.) referred to as SENTRYR POLYOX™ water-soluble resins, NF (National Formulary) grade. When higher molecular weights of polyethylene oxide are used, other hydrophilic agents, such as salts or sugars, like glucose, sucrose, or lactose, that promote erosion or disintegration of this coating, are also included.

The time-delayed dosage form can be a mechanical pill such as an Enterion® capsule or pH sensitive capsule which can release the drug after a pre-programmed time or when it receives a signal which can be transmitted or once it leaves the stomach.

The amount of the compound of the disclosure in a formulation can vary within the full range employed by those skilled in the art. Typically, the formulation will contain, on a weight percent (wt %) basis, from about 0.01-99.99 wt % of the RIPK1 Inhibitor based on the total formulation, with the balance being one or more suitable pharmaceutical excipients. In one embodiment, the compound is present at a level of about 1-80 wt %.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

### EXAMPLES

The following examples are provided to illustrate certain disclosed embodiments and are not to be construed as limiting the scope of this disclosure in any way.

### Example 1—Treatment of Coronavirus Patients with a RIPK1 Inhibitor

The RIPK1 Inhibitor is desirably used as a rescue treatment for patients who have a potentially detrimental immune response to SARS-CoV-2. Target population should be patients who have manifested with signs and symptoms associated with an exaggerated immune response to SARS-CoV-2, including clinical status (e.g., oxygen requirement), relative lymphopenia, elevated IL-6, Hscore for cytokine storm, i.e., patients who have a clinical “picture” consistent with a hyperinflammatory state/SIRS path, potentially with looming cytokine storm. Current conventional thinking is that early intervention (asymptomatic or mild symptoms only) is not recommended, given that RIPK1 inhibition could interfere with interferon signaling which is needed in early antiviral response and may interfere with a normal host response.

The RIPK1 Inhibitor is intended to treat severe coronavirus infection patients at risk of SIRS, which is the most common cause of death in coronavirus infections, such as COVID-19 infections. RIPK1 inhibition is not known to have antiviral activity, but is expected to be complementary to antiviral therapy by preventing or reducing the severity of the SIRS, which is responsible for most of the mortality associated with coronavirus infection. Since early in the disease—a phase dominated by virus replication—RIP kinase inhibition may be counterproductive, therefore, administration of the RIPK1 Inhibitor is, in an embodiment, done once laboratory assessments and biomarkers suggest a strong innate immune response. Based on mechanism of action, the RIPK1 Inhibitor may have broader effects than IL-6-receptor blockade inhibiting apoptosis/necroptosis, TNF-α and interferon pathways. Treatment duration may be variable and is planned to continue until markers of inflammation are reduced and oxygenation improves. In an embodiment, a 300 mg BID dose of the RIPK1 Inhibitor, followed by a dose reduction (150 mg) to minimize the risk of a rebound effect, is administered to the patient. The desired route of administration of the RIPK1 Inhibitor is orally, e.g., in capsule form, but administration through an oral nasal feeding tube may resorted to for patients requiring mechanical ventilation.

A study to test the RIPK1 Inhibitor in human patients is set forth herein. The study is a 60 day (28 days on treatment) randomized placebo-controlled parallel group study in patients with severe coronavirus infections at risk for SIRS. During the hospital stay, patients will be assessed daily; patients discharged from hospital will be followed up on Day 60 either in person or by phone. A Phase 2 part of the study can include 60 patients on the RIPK1 Inhibitor and 40 patients on placebo, Phase 3 can include 120 patients on the RIPK1 Inhibitor and 60 patients on placebo (sample sizes approximate; will have to be confirmed by statistical line function). The study has an adaptive design permitting changes of the inclusion-/exclusion criteria, endpoints and a sample size re-estimation upon completion of the Phase 2 part.

Study Description

Design: Adaptive, randomized, placebo-controlled 60-day study to assess efficacy and safety of 300 mg BID of the RIPK1 Inhibitor followed by 150 mg once daily in hospitalized patients with severe coronavirus infection at risk of SIRS.

Patient Population:
Males and females, 18 to 80 years of age
Confirmed infection with 2019-nCoV/SARS-CoV-2
Severe disease with dyspnea, requirement of oxygen support, evidence of pneumonia, either radiographic or on auscultation (may permit enrollment of critical patients based on Phase 2 results)
Hospitalized or planned to be admitted
Relative Lymphopenia

Treatment:

The RIPK1 Inhibitor 300 mg BID oral capsules followed by 150 mg BID or matching placebo on top of usual care. The treatment can be given on top of antiviral therapy. In ventilated patients, the RIPK1 Inhibitor will be administered by gastric feeding tube.

Treatment will be initiated upon laboratory and biomarker changes indicating innate immunity activation such as increase in CRP, decreasing neutrophil numbers, increase in IL-6, exact parameters TBD.

Primary Endpoint:
change in CRP concentration over baseline compared to placebo

Secondary Endpoints
Key secondary endpoint: ventilator free days and alive within the 28-day study window
Time to end of oxygen support/oxygen saturation/FiO₂>=92% breathing room air (starting at the initiation of study treatment)
Time to resolution of fever—≤36.6° C. (axilla) or ≤37.2° C. (oral), or ≤37.8° C. (rectal or tympanic)
7-point clinical scale, daily assessments (1. Death; 2. Hospitalized, on invasive mechanical ventilation or ECMO; 3. Hospitalized, on non-invasive ventilation or high flow oxygen devices; 4. Hospitalized, requiring supplemental oxygen; 5. Hospitalized, not requiring supplemental oxygen—requiring ongoing medical care (coronavirus related or otherwise); 6. Hospitalized, not requiring supplemental oxygen—no longer requires ongoing medical care; 7. Not hospitalized assessed over a 30 and 60 day period
Days in the ICU alive
Days in hospital alive
Incidence of other organ failures and or sepsis, percentage of patients meeting ALI or ARDS criteria
All-cause mortality

### Example 2—Clinical Trial to Study Treatment of Coronavirus-Infected Patients with a RIPK1 Inhibitor

Coronavirus disease 2019 (COVID-19) is caused by severe acute respiratory syndrome coronavirus 2 (SARS-CoV-2), a protein-enveloped RNA virus (1) related to severe acute respiratory syndrome coronavirus (SARS-CoV) and Middle East respiratory syndrome coronavirus (MERS-CoV) (2). COVID-19 presents with influenza-like symptoms (e.g., fever, cough, dyspnea, nausea, vomiting, diarrhea) and radiographic features of diffuse pneumonia (3, 4, 5, 6), with more severe cases characterized by neutrophilia or neutropenia, lymphopenia, thrombocytopenia, elevations in acute phase reactants and inflammatory cytokines (5). Over 25% of severe cases develop acute respiratory distress during the second week of hospitalization (4). Acute, life-threatening respiratory injury induced by coronavirus infection is thought to be associated with an over-exuberant cytokine release (also known as “cytokine storm”) (7, 8).

Case series of patients afflicted with SARS-CoV and MERS-CoV pneumonia indicate that elevations in interleukin (IL)-6 and other pro-inflammatory cytokines are correlated with clinical and radiographic severity (9, 10), and that in SARS-CoV pneumonia, peak viral load precedes peak IL-6 concentration and subsequent peak radiographic severity (11). In contrast to autopsies from patients who died from ARDS secondary to influenza A (H1N1), autopsies from patients who died from COVID-19 showed pulmonary vascular endotheliosis, thrombosis and angiogenesis (12). Currently, no therapeutics against COVID-19 have demonstrated meaningful efficacy.

Receptor interacting serine/threonine protein kinase 1 (RIPK1) is an intracellular protein that can be found in the downstream signaling pathways of tumor necrosis factor (TNF) family receptors, toll-like-receptors (TLR) 3 and 4 as well as interferon receptors. Two main functions of RIPK-mediated cell signaling are executed via the scaffolding properties important in the nuclear factor-kappa B signaling pathway to promote cell survival and inflammation, and the kinase function involved in regulating the necroptotic cell death pathway after various stimuli.

Published data have suggested that both RIPK1 kinase-driven inflammation and cell death are key contributing factors to TNFα-induced systemic inflammatory response syndrome (SIRS) (13, 14, 15, 16). In addition, other studies suggested that RIPK1 kinase inhibition may suppress vascular system dysfunction and endothelial/epithelial cell damage in addition to exacerbated inflammatory signaling (14, 17). As RIPK1 is considered a master regulator of cell death and inflammation, it was hypothesized that selectively targeting its kinase activity could mitigate the devastating sequelae of the hyperinflammatory state observed in late stage severe cases of COVID-19.

The RIPK1 Inhibitor is a highly potent, selective oral inhibitor of RIPK1 activity under development for immunomodulatory rescue treatment for severe COVID-19 and autoimmune skin diseases. It is proposed to target severe and critical COVID-19 patients at increased risk for SIRS.

Clinical data from the first-in-human (FIH) studies in healthy volunteers have demonstrated that RIPK1 Inhibitor was safe and well tolerated with doses ranging from 10 mg to 800 mg single dose and 50 mg to 600 mg repeated daily doses over 2 weeks. Non-human primate toxicology studies up to 29 days and up to 500 mg/kg/day also did not raise any safety concerns.

This study was designed to evaluate the safety and immunomodulatory effect of the RIPK1 Inhibitor compared to placebo in hospitalized adults with severe COVID-19. The knowledge gained from this study could significantly inform a larger follow-up trial to demonstrate a clinically significant effect of RIPK1 inhibition in COVID-19.

The primary objective of the study was:
to evaluate the effect of the RIPK1 Inhibitor relative to the control arm on the hyperinflammatory state as measured by C-reactive protein (CRP) levels in adult patients hospitalized with severe COVID-19.

The secondary objectives of the study were as follows:

Main secondary objectives were:
to evaluate the time to onset of effect of the RIPK1 Inhibitor relative to the control arm on the hyperinflammatory state as measured by CRP levels
to evaluate the time to onset of effect of the RIPK1 Inhibitor relative to the control arm on oxygenation status
to evaluate the effect of the RIPK1 Inhibitor relative to the control arm on oxygenation status

Other secondary objectives were:
to evaluate the effect of the RIPK1 Inhibitor relative to the control arm on total duration of supplemental oxygen requirement
to evaluate the effect of the RIPK1 Inhibitor relative to the control arm on length of ventilator support needed
to evaluate the effect of the RIPK1 Inhibitor relative to the control arm on laboratory markers of severe COVID-19
to evaluate the effect of the RIPK1 Inhibitor relative to the control arm on mortality
to evaluate the effect of the RIPK1 Inhibitor relative to the control arm on need for thrombolytic therapy
to evaluate the effect of the RIPK1 Inhibitor relative to the control arm on need for vasopressor treatment
the secondary safety objectives of the study are to evaluate the safety of the RIPK1 Inhibitor as compared to the control arm up to End of Study
to evaluate the effect of the RIPK1 Inhibitor relative to the control arm on total duration without high flow supplemental oxygen requirements.

The exploratory objectives of this study were:
to evaluate the effect of the RIPK1 Inhibitor relative to the control arm on exploratory clinical laboratory markers of severe COVID-19
to evaluate differences in categorical outcomes between the treatment and the control arm
to evaluate time to improvement in categorical outcomes between the treatment and the control arm
to evaluate the cytokine profile and additional biomarkers that may be associated with efficacy and safety associated with RIPK1 Inhibitor treatment
to evaluate the effect of the RIPK1 Inhibitor compared to the control arm on detectable viral load in plasma in severe COVID-19 participants
to evaluate the pharmacokinetic (PK) exposure of the RIPK1 Inhibitor in participants with severe COVID-19.

A list of abbreviations and definitions of terms is provided herein:

AE: adverse event

AESI: adverse event of special interest

ALT: alanine aminotransferase

BID: twice a day

BLOQ: below limit of quantitation

COVID-19: coronavirus disease 2019

CRP: C reactive protein

CV: coefficient of variance

CYP: cytochrome P450

ECG: electrocardiogram

eCRF: electronic case report form

EOT: end of treatment

FIH: first-in-human

FiO₂: fraction of inspired oxygen

HLGT: high level group term

HLT: high level term

IL: interleukin
IMP: investigational medicinal product
KM: Kaplan-Meirer
LDH: lactate dehydrogenase
LOCF: last observation carried forward
LS: least square
MedDRA: Medical Dictionary for Regulatory Activities
MERS-CoV: Middle East respiratory syndrome-related coronavirus
MMRM: Mixed model with repeated measures
PCSA: potentially clinically significant abnormality
PK: pharmacokinetic
PT: preferred term
RBC: red blood cell
RFFD: Respiratory Failure-Free Days
RIPK1: receptor interacting serine/threonine protein kinase 1
RT-PCR: reverse transcription polymerase chain reaction
SAE: serious adverse event
SAP: statistical analysis plan
SARS-CoV: severe acute respiratory syndrome coronavirus
SARS-CoV-2: severe acute respiratory syndrome coronavirus 2
SD: standard deviation
SEM: standard error of the mean
SIRS: systemic inflammatory response syndrome
SpO₂: saturated oxygen
TLR: toll-like receptor
TNF: tumor necrosis factor
WBC: white blood cell
WOCBP: women of child bearing potential

### 1. INVESTIGATIONAL PLAN

1.1. Description of Overall Study Design and Plan

This study was a multinational, multi-center, double-blind, 2:1 randomized (RIPK1 Inhibitor to placebo), placebo-controlled study in adult participants hospitalized for severe COVID-19.

The study included 3 periods:
A maximum 4-day screening period;
A maximum 15-day treatment period (including one end of treatment [EOT] day);
A minimum of 13-day post-intervention observation period.

Approximately 72 participants were targeted for enrollment to achieve 67 participants randomized to receive RIPK1 Inhibitor or Placebo in addition to local standard of care, for an expected number of 60 evaluable participants (40+20). Randomization was stratified by site.

1.2. Discussion of Study Design and Choice of Control Groups

This Phase 1b study was designed as a small safety and proof-of-mechanism study aimed at testing the RIPK1 Inhibitor in a very targeted patient population to rapidly gather safety and disease-specific pharmacodynamic and clinical data. The population selected, hospitalized patients with severe COVID-19, had clear signs of immune activation to test the hypothesis that RIPK1 inhibition would ameliorate the deleterious inflammatory response.

In the absence of treatments with demonstrated efficacy, a placebo control was warranted to distinguish the safety and tolerability of the RIPK1 Inhibitor from the background signs and symptoms of COVID-19 infection as well as evaluate its potential to affect CRP and other markers of disease. While not powered to demonstrate efficacy, clinical assessments could demonstrate a reduction in oxygen requirements and/or need for intubation, among other secondary clinical outcomes.

This study utilizes a double-blind to minimize potential for bias on the part of the investigator, participant, or sponsor, but a 2:1 ratio to ensure that in case of benefit, the number of participants assigned to active treatment is increased.

A daily dose of 600 mg the RIPK1 Inhibitor was selected for this study was based on preclinical data and two FIH studies. The FIH studies demonstrated that the RIPK1 Inhibitor was safe and well tolerated after single oral doses up to 800 mg and at multiple daily doses up to 600 mg in healthy participants.

The duration of treatment of 14 days was supported by clinical safety, tolerability and target engagement in healthy participants. In addition, in other clinical studies participants with severe COVID-19 are often discharged from the hospital home by Day 15.

The knowledge gained from this study could significantly inform a larger follow-up trial to demonstrate a clinically significant effect of the RIPK1 inhibition in patients with COVID-19.

Participants were included in the study according to the following criteria.

1.2.1. Inclusion Criteria

Participants are eligible to be included in the study only if all of the following criteria apply:

Age
I 01. Participant (Male and Female) must be ≥18 years and ≤80 years of age inclusive, at the time of signing the informed consent.

Type of Participant and Disease Characteristics
I 02. Hospitalized (or documentation of a plan to admit to the hospital if the participant is in an emergency department) with evidence of COVID-19 related lung disease diagnosed by chest radiograph, chest computed tomography or chest auscultation (rales, crackles) AND with severe disease defined as follows:
   The participant requires oxygen supplementation administered by nasal cannula, simple face mask, or other similar oxygen delivery device (i.e., increase in oxygen requirement following SARS-CoV-2 infection). Participant should require no more than 40% FiO₂ and no more than 6 L/min of flow.
I 03. SARS-CoV-2 infection confirmed by RT-PCR, or other commercial or public health assay in any specimen, within 3 weeks prior to randomization, and no alternative explanation for current clinical condition.
I 04. At time of randomization, have demonstrated laboratory signs consistent with systemic inflammation: CRP>50 mg/L.
I 05. Willing and/or able to comply with study-related procedures/assessments.

Sex
I 06. Male and/or female participants, including women of childbearing potential (WOCBP). WOCBP must have a negative pregnancy test (highly sensitive urine or serum as required by local regulations) at screening and should agree to use an acceptable contraceptive method during treatment with the RIPK1 Inhibitor and for at least 5 days after treatment termination. Regional definitions for effective contraception will apply for each country.
I 07. Capable of providing signed informed consent which includes compliance with the requirements and restrictions listed in the informed consent form (ICF) and in this protocol.

1.2.2. Exclusion Criteria

Participants are excluded from the study if any of the following criteria apply:

Medical conditions and prior/concomitant therapy
E 01. In the opinion of the investigator, unlikely to survive after 48 hours, or unlikely to remain at the investigational site beyond 48 hours*. *Note: participants requiring extracorporeal life support, vasopressors, or renal replacement therapy at randomization are excluded.
E 02. Participants requiring use of invasive or non-invasive positive pressure ventilation at randomization.
E 03. Presence of any of the following abnormal laboratory values at screening: ALT greater than 5×ULN, platelets<50 000 per mm³, hemoglobin<9 g/dL.
E 04. Any prior (within the defined periods below) or concurrent use or plans to receive during the study period of immunomodulatory therapies (other than interventional drug) at screening including but not limited to the following:
   Anti-IL-6, anti-IL-6R antagonists or with Janus kinase inhibitors (JAKi) in the past 30 days prior to randomization.
   Cell-depletion agents (e.g., anti-CD20) without evidence of recovery of B cells to baseline level 30 days prior to randomization.
   Anakinra within 14 days of baseline.
   Abatacept within 60 days of baseline.
   Tumor necrosis factor (TNF) inhibitors within 14-60 days (etanercept within 14 days, infliximab, certolizumab, golimumab, or adalimumab within 60 days),
   Alkylating agents including cyclophosphamide (CYC) within 6 months of baseline.
   Cyclosporine (CsA), azathioprine (AZA) or mycophenolate mofetil (MMF) or methotrexate within 2 weeks of baseline.
   Intravenous immunoglobulin (IVIG) within the past 3 months or plans to receive during the study period.
   Convalescent serum.
E 05. Use of chronic systemic corticosteroids for a non-COVID-19-related condition in a dose higher than prednisone 10 mg or equivalent per day at screening.
E 06. Exclusion criteria related to tuberculosis (TB) and non-tuberculous mycobacterial (NTM) infections:
   Known active or history of incompletely treated TB or NTM pulmonary infection.
   Suspected or known extrapulmonary tuberculosis or NTM infection.
E 07. Participants with suspected or known active systemic bacterial or fungal infections within 4 weeks of screening.
E 08. Pregnant or breastfeeding women.
E 09. Unable to swallow the required number of capsules due to esophageal or GI disease and/or for other reasons, per judgment of the Investigator.
E 10. Current or chronic history of liver disease, or known hepatic or biliary abnormalities (with the exception of Gilbert's syndrome or asymptomatic gallstones)

Prior/Concurrent Clinical Study Experience
E 11. Participation in any clinical research study, including any double-blind study, evaluating an investigational product or therapy within 3 months and less than 5 half-lives of investigational product prior to the screening visit.

Other Exclusions
E 12. Participant who withdraws consent during the screening period (following signing of the informed consent form).
E 13. Any findings on physical examination or history of any illness that, in the opinion of the study investigator, might confound the results of the study or pose an undue risk to the safety of the participant.
E 14. Individuals accommodated in an institution because of regulatory or legal order; prisoners or participants who are legally institutionalized.
E 15. Participant not suitable for participation, whatever the reason, as judged by the Investigator, including medical or clinical conditions, or participants potentially at risk of noncompliance to study procedures.
E 16. Participants are employees of the clinical study site or other individuals directly involved in the conduct of the study, or immediate family members of such individuals.
E 17. Any specific situation during study implementation/course that may raise ethical concerns.
E 18. Sensitivity to any of the study interventions, or components thereof, or drug or other allergy that, in the opinion of the Investigator, contraindicates participation in the study.

1.3. Treatments

1.3.1. Treatments Administered

The investigational medicinal products (IMPs) administered in this study were the RIPK1 Inhibitor and matching placebo.

Participants were assigned to treatment according to randomization list. Six RIPK1 Inhibitor 50 mg capsules (300 mg) or matching placebo capsules were administered orally in fasting or fed conditions twice a day (BID). For participants intubated with feeding tube in place, the IMPs were given as suspension by feeding tube.

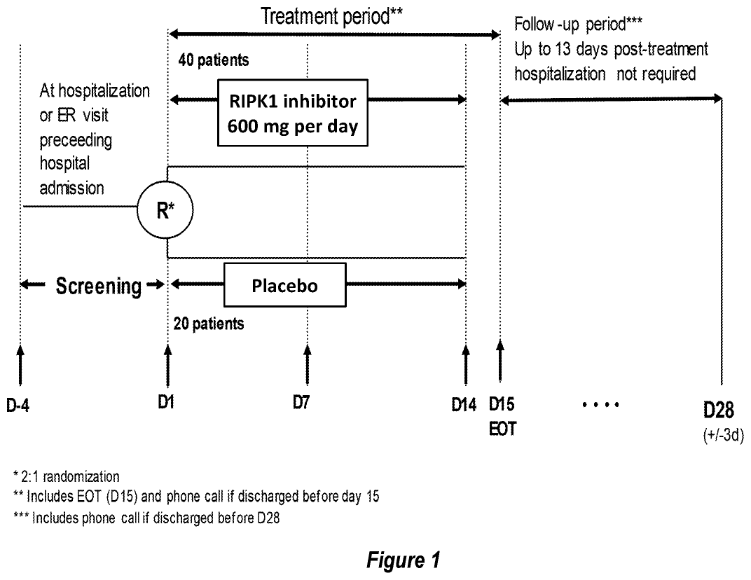

The study treatment was given from Day 1 to Day 14. The treatment duration of 14 days was selected based on the pre-clinical SIRS model derived rapid onset of action; in addition, in other clinical studies, participants with severe COVID-19 were often discharged from the hospital home by Day 15. See also FIG. 1.

1.3.2. Identity of Investigational Medicinal Products

The IMPs were provided by the Sponsor as identical capsules (hard gel) packaged in blister packs. The strengths and batch numbers used were the following:
RIPK1 Inhibitor: 50 mg
placebo

1.3.3. Method of Assigning Participants to Treatment Groups

A randomized participant was defined as a participant who had been allocated to a randomized intervention regardless of whether the intervention kit was used or not. A participant could not be randomized more than once in the study.

Participants who complied with all inclusion/exclusion criteria were assigned a participant number according to the chronological order of inclusion, and corresponding treatment was allocated according to the participant randomization list (stratified by site) generated centrally by an interactive response technology system.

Participants were randomized in 2:1 (RIPK1 Inhibitor to placebo) ratio to treatment arms. Study interventions corresponding to the participant treatment arm were dispensed at the study visit summarized in the study flowchart (Table 1).

TABLE 1
STUDY FLOWCHART

| | | Treatment period | | | | Follow-up | | |
|---|---|---|---|---|---|---|---|---|
| | | | | End of | | | End of Study, | |
| | | | | Treatment, or | | | or Discharge/ | |
| | | | | Discharge/ | | Post- | Early | Follow-up |
| | | | | Early | Follow | treatment | Discontinuation | call^{d} (if |
| Study period | | | | Discontinuation | up call (if | follow- | Day | Discharge |
| Study | Screening | | | up to Day 15 | Discharge | up | 16 to 28 | before |
| procedure | Screening^{a} | Intervention | | (EOT) | before | EOT | (EOS)^{c} | Day 28) |
| Day | D-4 to D-1 | D1 | D2-14 | D15^{e} | Day 15) | +1 to D27 | D28 | D28 |
| Window | | | | | | | | D28 ±3 |
| (day) | | | | | | | | |

Screening/Baseline

| | | |
|---|---|---|
| Eligibility | X | |
| Informed | X | |
| consent | | |
| Demographics | X | |
| Medical | X | |
| History | | |
| Randomization | (X)^{b} | X^{b} |
| Confirm | | X |
| eligibility | | |

Treatment

| Study drug | | D1-D14: | | | |
|---|---|---|---|---|---|
| administration | | 300 mg BID^{f, g} | | | |
| Log-in to IRT | X | X | X | X | X |

Assessments
Clinical assessments

| | | | | | | | | |
|---|---|---|---|---|---|---|---|---|
| Oxygen | X | X | Daily (until | X | | If | X | |
| delivery and | | | hospital | | | available | | |
| oxygenation^{h} | | | discharge) | | | | | |
| Resting | X | X^{m} | Daily (until | X | | If | X | |
| SpO₂ⁱ | | | hospital | | | available | | |
| | | | discharge) | | | | | |
| Clinical status | X | X | Daily (until | X | X | If | X | X |
| assessment | | | hospital | | | available | | |
| (including | | | discharge) | | | | | |
| 7-point | | | | | | | | |
| ordinal scale) | | | | | | | | |
| Vital status | | X | Daily (until | X | X | If | X | X |
| (and cause | | | hospital | | | available | | |
| of death) | | | discharge) | | | | | |
| Arterial | | X | If | If available | | If | If available | |
| blood gas | | | available | | | available | | |
| resultsⁱ | | | | | | | | |
| Vital signs | Xⁿ | X^{m} | Daily | X | | If | X | |
| (including body | | | (until | | | available | | |
| temperature, | | | hospital | | | | | |
| respiratory rate) | | | discharge) | | | | | |
| Targeted | X | X | Daily (until | X | | If | X | |
| physical | | | hospital | | | available | | |
| examination | | | discharge) | | | | | |
| (including lung | | | | | | | | |
| auscultation, | | | | | | | | |
| consciousness) | | | | | | | | |
| 12-lead | X | | As | X | | | | |
| Electro- | | | available per | | | | | |
| cardiogram | | | clinical care | | | | | |
| Record | X | X | Daily (until | X | X | <- X -> | X | X |
| concomitant | | | hospital | | | | | |
| therapy | | | discharge) | | | | | |
| Adverse | X | X | X | X | X | <- X -> | X | X |
| events^{k} | | | | | | | | |

Laboratory testing

| | | | | | | |
|---|---|---|---|---|---|---|
| Hematology^{l} | X | X^{m} | As | X | If | If available |
| | | | available per | | available | |
| | | | clinical | | | |
| | | | care at | | | |
| | | | minimum | | | |
| | | | D3, D5, D7 | | | |
| Blood | X | X^{m} | As | X | If | If available |
| chemistry^{l} | | | available per | | available | |
| | | | clinical | | | |
| | | | care at | | | |
| | | | minimum | | | |
| | | | D3, D5, D7 | | | |
| Serum or | X | | | If available | | If available |
| urinary | | | | | | |
| pregnancy | | | | | | |
| testing for | | | | | | |
| WOCBP | | | | | | |
| CRP^{l} | X | X^{m} | As | X | If | If available |
| | | | available per | | available | |
| | | | clinical | | | |
| | | | care at | | | |
| | | | minimum | | | |
| | | | D3, D5, D7 | | | |
| D-dimer^{l} | X | X^{m} | As | X | If | If available |
| | | | available per | | available | |
| | | | clinical | | | |
| | | | care at | | | |
| | | | minimum | | | |
| | | | D3, D5, D7 | | | |
| LDH^{l} | | X^{m} | As | As available | If | If available |
| | | | available per | per clinical | available | |
| | | | clinical | care | | |
| | | | care | | | |
| Ferritin^{l} | | X^{m} | As | As available | If | If available |
| | | | available per | per clinical | available | |
| | | | clinical | care | | |
| | | | care | | | |

PK/Biomarkers

| | | | |
|---|---|---|---|
| Samples | X | D3, D7, | |
| for RIPK1 | | D14 | |
| Inhibitor PK | | | |
| analyses^{o} | | | |
| Blood | X^{m} | D3, D5, | X |
| Samples for | | D7 | |
| cytokines and | | | |
| chemokines | | | |
| biomarker | | | |
| analysis | | | |
| Blood for | X^{m} | D3, D7 | X |
| RT- PCR | | | |
| SARS-CoV- | | | |
| 2 (optional) | | | |
| Blood | X^{m} | | X |
| Samples | | | |
| for genetic | | | |
| analysis | | | |
| (optional) | | | |

EOT: End of treatment, EOS: end of study, CRP: C-reactive protein, LDH: Lactate dehydrogenase, PK: pharmacokinetic, RT-PCR: reverse transcription polymerase chain reaction, SARS-CoV-2: severe acute respiratory syndrome coronavirus 2, SpO₂: oxygen saturation, WOCBP: women of childbearing potential.
^{a}Screening visit allowed for enrollment of participant; randomization was triggered by CRP >50 mg/L.
^{b}Randomization could occur rapidly after screening if feasible; however, dosing was to start in the morning (before 12.00 noon; if randomized in afternoon, dosing was started next morning).
^{c}For participants who completed the treatment period: EOS assessments were done on day of early Discontinuation/Discharge if occurring between Day 16 to Day 27, or on Day 28 (whichever was earlier).
^{d}Participants discharged before Day 28 were to receive a follow-up phone call (at Day 28 ±3 days) (or more frequently if necessary/applicable depending on site management) to collect health status, safety data and history of hospital re-admission (if applicable).
^{e}EOT assessments were done on day of early Discontinuation/Discharge if occurring between Day 1 to Day 15, or on Day 15 if participant remained hospitalized and continued in the study.
^{f}Treatment dose: 300 mg PO BID up to and including Day 14. In case participants were discharged from the hospital before Day 14, treatment was to be discontinued before discharge and EOT assessments were performed on day of discharge.
^{g}If participant was intubated during treatment period, treatment could be given as suspension via feeding tube.
^{h}Delivery device and flow to calculate FiO₂ or use FiO₂ taken from the ventilator were to be recorded.
ⁱTest were to be measured after 5 minutes of rest (sitting or supine) and (when applicable) and simultaneously with oxygen delivery and ventilation data.
^{j}Results as reported were recorded in arterial blood gas results electronic Case Report Form (eCRF).
^{k}All Aes were recorded in CRF.
Note:
any abnormal physical findings requiring medical or surgical intervention were recorded as an AE.
^{l}—
^{m}Pre-dose assessment.
ⁿAt screening only: including height and weight.
^{o}Samples for RIPK1 Inhibitor PK analyses were to be collected at the following timepoints: Day 1: PK sampling within 2 to 5 hours after the first morning dose (around Cmax); Day 3 PK sample just before or within 1 h before the morning dosing; Day 7 and Day 14: PK sample just before or within 1 hour of the morning dose (Ctrough) and within 2-5 hours after the morning dose if possible. If discharged before Day 14: PK samples within 1 hour before the last dose and before discharge.

1.3.4. Blinding Procedures

RIPK1 Inhibitor 50 mg and matching placebo were provided in identically and visually indistinguishable capsules. Blisters and box were labeled with a treatment kit number.

In case the intervention was to be administered by feeding tube, unblinded qualified site personnel were to prepare a suspension and to ensure that administering personnel remained blinded. With the exception of the unblinded site personnel described above, the Investigator and other staff members in charge of the participant, and the participants were to remain blinded.

The Investigator, the study site and Sponsor's clinical trial team members did not have access to the randomization (treatment) code except under circumstances described in the protocol.

1.3.5. Prior and Concomitant Therapy

The prohibited prior and concomitant medications in this study were described in exclusion criteria for description of medications that were not to be used prior to inclusion.

In addition to the prohibited immunomodulatory therapies, concomitant use of strong inducers of cytochrome P450 (CYP) enzyme CYP3A4 and CYP1A should be avoided due to their potential to reduce RIPK1 Inhibitor exposure.

1.4. Efficacy/Pharmacodynamics, Safety, and Pharmacokinetics Assessments

An overview of efficacy/PD, safety, and PK assessments relative to study procedures is presented in Table 1.

The effect of RIPK1 Inhibitor relative to the placebo arm was evaluated based on the changes of background signs and symptoms of COVID-19 infection, as well as on the changes in hyperinflammatory status as measured by CRP level and other markers of disease.

The clinical assessment in this study included both the assessment of clinical laboratory variables (CRP, laboratory markers of severe COVID-19 [D-Dimer, hematology parameters and thrombolytic therapy and vasopressor treatment]), oxygenation variables (saturated oxygen [SpO₂], SpO₂/fraction of inspired oxygen [FiO₂] ratio), and clinical status variables (7-point clinical scale). The pharmacodynamic assessment included the measurement of peripheral biomarkers (pro-inflammatory cytokines and RIPK1 PD cytokines/chemokines), and optional measurement of viral load of SARS-CoV-2.

Further details of assessments are described in subsections that follow.

1.5. Efficacy/Pharmacodynamics Assessments

1.5.1. Efficacy/Pharmacodynamics Measurements and Timing

For clinical assessment, the variables associated with endpoints were:
main inflammatory marker CRP
Oxygenation saturation and oxygen delivery (e.g. SpO₂, SpO₂/FiO₂), Laboratory markers of severe COVID-19 including D-dimer, lactate dehydrogenase (LDH), ferritin and hematology laboratory (white blood cell count, differential blood lymphocytes, neutrophil to lymphocyte ratio)
Clinical status of participant (7-point ordinal scale)
Thrombolytic and vasopressor treatments

The biomarker variables included pro-inflammatory cytokines (such as IL 4, IL-6, IL-10, IL-17, TNFα, and IFNγ) and RIPK1 PD cytokines/chemokines (such as MIP1α and MIP1β) that are elevated in participants with SARS-CoV-2.

1.5.1.1. Primary Clinical Assessment Variable

The primary clinical assessment endpoint was the relative change from baseline in CRP level on Day 7.

1.5.1.2. Secondary Clinical Assessment Variables

The main secondary clinical assessments endpoints included:
Time to 50% decrease from baseline in CRP level
Time to improvement of oxygenation as measured by oxygen saturation≥92% breathing room air over 48 hours or until discharge
Change from baseline in SPO₂/FiO₂ ratio at Day 7

Other secondary clinical assessment endpoints included:
Number of Days without need for oxygen support and alive (oxygen saturation≥92% breathing room air) up to Day 28
Numbers of Ventilator-free days and alive up to Day 28
Change from baseline in markers of inflammation (White blood cell count, differential blood lymphocytes, neutrophil to lymphocyte ratio, IL-6) and D-Dimer at Day 7 and EOT
Incidence of Deaths up to Day 28
Percentage of participants receiving thrombolytic treatment up to Day 28
Percentage of participants receiving vasopressor treatment up to Day 28
Numbers of Respiratory Failure-Free Days (RFFD) and alive up to Day 28

1.5.1.3. Exploratory Clinical Assessment and Biomarker Variable

Exploratory clinical assessments endpoints included:
Change from baseline in ferritin and LDH at Day 7 and EOT
Proportion of participants per category of the 7-point clinical scale at EOT
Time to improvement by 2 points in category of 7-point clinical scale
Quantitative SARS-COV-2 viral load in blood at baseline and on Day 3, 5, 7 and EOT

The 7-point clinical scale is described below:
1. Death
2. Hospitalized, on invasive mechanical ventilation or ECMO
3. Hospitalized, on non-invasive ventilation or high flow oxygen devices
4. Hospitalized, requiring supplemental oxygen
5. Hospitalized, not requiring supplemental oxygen—requiring ongoing medical care (COVID-19 related or otherwise)
6. Hospitalized, not requiring supplemental oxygen—no longer requires ongoing medical care
7. Not hospitalized

The exploratory PD/biomarker endpoint was the change from baseline in peripheral cytokine and biomarker levels up to EOT.

1.5.1.4. Adverse Events

Safety evaluation was based on adverse events (Aes) including serious adverse events (SAEs) and adverse events of special interest (AESIs) (i.e., pregnancy, symptomatic overdose with IMP. Alanine aminotransferase [ALT] increase, and anemia), and treatment-emergent adverse events (TEAEs) leading to treatment discontinuation.

1.5.1.5. Laboratory Safety Parameters

Standard clinical laboratory parameters (hematology, blood chemistry) were measured per protocol.

1.5.1.6. Other Safety Parameters

Physical examination including lung auscultation and assessment of consciousness, vital sign, electrocardiogram (ECG) parameters were measured per protocol.

1.5.2. Pharmacokinetics Assessments and Timing

1.5.2.1. Pharmacokinetic Variables

RIPK1 Inhibitor concentrations at selected time points over the two weeks of treatment were summarized by descriptive statistics. PK parameters such as Cₘₐₓ, tₘₐₓ, and AUC were calculated by a Bayesian analysis: the main results are presented in Section 5.2.

1.5.2.2. Appropriateness of Measurements

Standard measurements appropriate for the analyses of the safety and PK variables of RIPK1 Inhibitor were used in this study.

There are no proven treatments available for patients who have infection with SARS-CoV-2. The clinical assessment chosen in the study were based on the knowledge of the disease-specific mechanisms to test the effect of RIPK1 Inhibitor on the systemic inflammatory changes and those in the lungs in particular.

The pro-inflammatory biomarker variable measured in the study included pro-inflammatory cytokines (such as IL-4, IL-6, IL-10, IL-17, TNFα, and IFNγ), and RIPK1 PD cytokines/chemokines (such as MIP1α and MIP1β) that have been observed to be elevated in patients with SARS-CoV-2 infection. Each analyte was selected, and the assay analytically validated based on reports in the literature and in-house research.

1.6. Data Quality Assurance

The Sponsor conducted Investigator meetings and training sessions for clinical research associates as well as individual site initiation meetings to develop a common understanding of the clinical study protocol, case report form, and study procedures, in compliance with GCP.

Regular site monitoring ensured the quality of trial conduct.

Monitoring of all investigator sites was performed by Sponsor staff according to Sponsor procedures.

Management of clinical trial data was performed according to the following rules and procedures. Data entry, verification and validation were carried out using a standard validated electronic data capture computer software (Medidata RAVE® version 2018.1.3 from study start to 10 Oct. 2020, Medidata RAVE® version 2020.2.0 from 10 Oct. 2020 to database lock). Data entry was performed directly from the Investigator site from the data source documents and signed electronically by the authorized site personnel. Moreover, any modification in the database was tracked using an audit trail.

1.7. Statistical Considerations

The following sections describe final analyses related to primary and main secondary objectives of the study.

1.7.1. Statistical Analyses

1.7.1.1. Analyses of Efficacy/Pharmacodynamic Endpoints

1.7.1.1.1. Analyses of Primary Pharmacodynamic/Biomarker Endpoints

The primary analysis on the relative change from baseline in CRP at Day 7 was based on a linear mixed model with repeated measurements (MMRM) fitted on log-relative change from baseline for Days 3, 5, 7 and 15. The model included fixed effects for participant-specific baseline log-CRP, visit, treatment group, and visit-by-treatment group interaction, and random effects for sites. Repeated measurements for each visit were taken within participant assuming an unstructured covariance pattern within treatment group.

The Least Square (LS) means of the relative change from baseline in CRP for the SAR group and placebo and corresponding 90% Cis were reported as geometric means. The difference in LS means at Day 7 (obtained on log-scale) and its confidence interval were exponentiated to provide an estimate of the geometric means ratio and corresponding 90% confidence interval. The one-sided p-value corresponding to testing if this ratio is ≥1 was reported.

The point estimate of the relative change from baseline in CRP and the difference between treatment groups, jointly with two-sided 90% confidence interval were reported for Days 3, 5, EOT. Time profile plots of point estimates of the relative change from baseline in CRP (+/−90% Cis) were presented by treatment group.

Missing values for the relative change from baseline in CRP for Days 3, 5, 7 and 15 in the primary analysis were replaced following the last observation carried forward (LOCF) approach, regardless if occurring before or after discontinuation/discharge/death. In case no LOCF could be identified, the missing value was not imputed. A sensitivity analysis was performed by repeating the above analysis without any imputation of missing values.

1.7.1.1.2. Analyses of Secondary Efficacy/Pharmacodynamic Endpoints

Efficacy parameters (without and with imputation where applicable) were summarized with descriptive statistics by treatment group per study day. Changes from baseline were summarized where applicable.

Profiles over study day were generated for individual values and treatment means (or median—interquartile range, boxplot) as appropriate.

When appropriate, scatterplots by treatment were generated to explore association between selected endpoints.

1.7.1.1.2.1. Time to Improvement in Crp

The time to 50% decrease relative to baseline in CRP level was estimated using the Kaplan-Meier (KM) approach. Earliest percent change from baseline<−50% in CRP was considered as event. Event times for participants in whom such a decrease was not observed was to be censored at the time point of the last observation collected. For participants who died during the study without experiencing the event, the last observation collected was carried forward to the longest duration of follow-up for any participant, plus 1 day. No sensitivity analysis was performed by also applying this last censor rule to participants with no event who were lost-to-follow-up, because no lost-to-follow-up were identified.

Summary table of the cumulative incidence rate over time and the cumulative incidence curves was provided by treatment arm.

The number and percentage of participants who experienced the event without applying censoring rules were reported at Days 3, 5, 7, 15 and 28.

Treatment arms were compared in an exploratory fashion using the log-rank test.

1.7.1.1.2.2. Time to Improvement Of Oxygenation

The time to improvement of oxygenation as measured by oxygen saturation>92% breathing room air over 48 hours or until discharge was estimated using the Kaplan-Meier approach and treatment arms were compared in an exploratory fashion using the log-rank test.

Presence of SO₂≥92% without use of any supplemental oxygen device on two consecutive days (earliest occurrence) or at day of discharge was considered as event. If such criterion was not met, time to event was censored at the time point of the last observation of SpO₂ collected. For participants who died during the study without experiencing the event, similar LOCF approached was used and a sensitivity analysis was performed as described in Section 1.7.1.1.2.1.

The number and percentage of participants who experience the event without applying censoring rules was reported at Days 3, 5, 7, 15 and 28.

1.7.1.1.2.3. SPO₂/FIO₂ Ratio

The analysis of the change from baseline in SpO₂/FiO₂ ratio was based on a MMRM model fitted on observed values for Days 2, 3, 4, 5, 6, 7 and 15. The model included fixed effects for participant-specific SpO₂/FiO₂ ratio at baseline, respective visit, treatment group, and visit-by-treatment group interaction, and random effects for sites. Repeated measurements for each visit were taken within participant assuming an unstructured covariance pattern within treatment group.

The LS means for the difference in change from baseline at Day 7 between RIPK1 Inhibitor and placebo were provided, jointly with the corresponding 90% confidence interval.

The point estimate of the change from baseline in SpO₂/FiO₂ ratio and the difference between treatment groups and two-sided 90% confidence interval value were reported for Day 2 to 7 and EOT as described above. Time profile plots of point estimates of the change from baseline (+/−90% Cis) were presented by treatment group.

Missing values for the change from baseline in SpO₂/FiO₂ ratio were replaced following the LOCF approach, regardless if occurring before or after discontinuation/discharge/death. In case no LOCF could be identified (e.g., no post-baseline value prior to Day 2 to replace a missing Day 2 result), the missing value was not imputed. A sensitivity analysis was performed by repeating the above analysis without any imputation of missing values.

1.7.1.2. Analyses of Safety Data

Adverse Events

The primary focus of AE reporting was on treatment emergent adverse events (TEAEs). Treatment emergent adverse events were Aes that were not present at baseline or represented the exacerbation of a pre-existing condition during the on-treatment period (treatment-emergent period), defined as the time from the first administration of the IMP up to and including the day of last dose of study drug plus 5 days.

All adverse events were coded to a “preferred term (PT)”, “high-level term (HLT)”, “high-level group term (HLGT)”, and associated primary SOC using Medical Dictionary for Regulatory Activities (MedDRA) version 23.1.

The number and cumulative incidence rate of deaths [%] during the on-study period were computed by treatment group: number of deaths divided by the number of participants. Kaplan-Meier plot for time to death was presented by treatment group.

Clinical Laboratory Evaluation, Vital Signs and Electrocardiogram

For laboratory parameters (hematology, clinical chemistry, and urinalysis), vital signs, and ECG, incidences of potentially clinically significant abnormality (PCSA) values, actual values and change from baseline were summarized by treatment group.

For all laboratory, vital signs and ECG parameters, raw data and change from baseline were summarized in descriptive statistics by treatment group and scheduled time of measurement, with the exception of AST, ALT and alkaline phosphatase: instead of summarizing data in descriptive statistics, participants' profiles were presented through graphics by treatment group and with a color code to identify sites. The reason was that blood samples were processed by local laboratories with different normal ranges. For the rest of clinical laboratory these parameters it is reasonable to pool data as they are standard procedure and no significant differences in normal ranges were expected.

1.7.1.3. Analyses of Pharmacokinetic Data

Descriptive statistics on plasma concentration of RIPK1 Inhibitor were analyzed by the Sponsor's Biostatistics Department.

Plasma concentrations of RIPK1 Inhibitor was listed and summarized by arithmetic mean, geometric mean, standard deviation (SD), standard error of the mean (SEM), coefficient of variance (%) (CV), minimum, median, maximum, and number of observations by timepoints. When applicable, relevant data were summarized by route: i.e., oral and oral gavage, and timepoints.

1.7.1.4. Pharmacokinetic/Clinical Assessments Analysis

Scatterplots were provided for clinical assessment data: e.g., CRP, SpO₂/FiO₂ versus PK plasma concentration when relevant.

### 2. STUDY PARTICIPANTS

2.1. Disposition of Participants

A total of 82 participants were screened, of which 67 were randomized and treated. The reasons for screen failure were predominantly based on criteria for inclusion/exclusion from the study (Section 1.2).

Of the 67 participants (with 20 participants received placebo and 47 participants received RIPK1 Inhibitor 600 mg), 51 discontinued the study treatment (14 in the placebo group and 37 in the RIPK1 Inhibitor group). Forty-five of 67 (67.2%) participants discontinued treatment early due to COVID-19 recovery with similar proportions between the placebo (13 of 20 or 65.0% participants) and RIPK1 Inhibitor arm (32 of 47 or 68.1% participants) (Table 3).

TABLE 3
Participant disposition

| | | RIPK1 Inhibitor |
|---|---|---|
| Status | Placebo | 600 mg |
| Randomized and treated | 20 | 47 |
| Did not complete the study treatment | 14 | 37 |
| period | | |
| Participant's decision for treatment | 0 | 1 |
| discontinuation | | |
| Reason for treatment discontinuation | | |
| Adverse Event | 1 | 1 |
| Progressive Disease | 0 | 2 |
| Recovery | 13 | 32 |
| Other ^{a} | 0 | 1 |
| Did not complete the follow-up | 2 | 2 |
| period | | |
| Reason for Follow-up discontinuation | | |
| Adverse Event | 2 | 2 |

^{a} verbatim terms for these discontinuations are provided in the “listing of participants with treatment discontinuation”
All randomized and treated participants started the follow-up period

2.2. Protocol Deviations

2.2.1. Major or Critical Deviations Potentially Impacting Efficacy Analyses

Major protocol deviations related to the primary clinical assessment endpoints were reported in a small percentage of participants and were balanced across the two treatment arms with no apparent distribution pattern (Table 4).

Overall, 7 participants received protocol-prohibited therapy as rescue therapy for the treatment of COVID-19 related complication.

Rescue medications including anti-IL-6 receptor antagonists or with Janus kinase inhibitors were given to 2 participants in the placebo group and 4 participants in the RIPK1 Inhibitor group.
Participants receiving the rescue medication on or before study Day 2 were excluded from the efficacy population
Participants who received anti-IL-6 rescue medicine after Day 2 visit were kept in the efficacy population, with assessments performed after use of the rescue medicine excluded from efficacy analysis.

One participant in the RIPK1 Inhibitor group received convalescent plasma to treat COVID-19 before the last IMP administration. According to the protocol, the IMP was to be discontinued immediately if a rescue therapy was administered (including convalescent plasma). The deviation was notified and discussed with PI and this participant was removed from efficacy population. Of note, this participant reported another major protocol deviation related to inclusion/exclusion criteria, who was in the opinion of the investigator, unlikely to survive after 48 hours or unlikely to remain at the investigational site beyond 48 hours.

One participant did not meet inclusion criteria for CRP level at the time of randomization, the case was considered a major protocol deviation and the participant was subsequently removed from the efficacy population.

TABLE 4
Critical or major deviations potentially impacting efficacy analyses

| | | RIPK1 Inhibitor |
|---|---|---|
| | Placebo | 600 mg |
| | (N = 21) | (N = 47) |
| Any protocol deviations potentially | 2 (9.5) | 6 (12.8) |
| impacting efficacy analysis | | |
| At time of randomization, have | 0 | 1 (2.1) |
| demonstrated laboratory signs consistent | | |
| with systemic inflammation: CRP >50 | | |
| mg/L. | | |
| In the opinion of the investigator, | 0 | 1 (2.1) |
| unlikely to survive after 48 hours, or | | |
| unlikely to remain at the investigational | | |
| site beyond 48 hours. See Note as per | | |
| protocol | | |
| Protocol prohibited | 2 (9.5) | 5 (10.6) |
| therapy/medication/vaccine administered | | |

Note:
Percentages are calculated using the number of participants randomized as denominator

2.2.2. Other Critical or Major Protocol Deviations

Other major deviations are summarized in Table 5.

Three participants from RIPK1 Inhibitor group had major protocol deviations due to late reporting of Aes.

One participant from the RIPK1 Inhibitor group reported a major protocol deviation in the informed consent procedures. By mistake, Director Delegate signed as a Director Delegate and also as an Impartial Witness on the main ICF for this.

TABLE 5
Other critical or major protocol deviations

| | | RIPK1 Inhibitor |
|---|---|---|
| | Placebo | 600 mg |
| | (N = 21) | (N = 47) |
| Any other important | 0 | 4 (8.5) |
| protocol deviation ^{a} | | |
| Failure to report | 0 | 3 (6.4) |
| AE/AESVSAE/Pregnancy/ | | |
| Overdose to sponsor | | |
| within the protocol- | | |
| specified time window | | |
| Informed consent/Assent form | 0 | 1 (2.1) |
| obtained with a misconduct in | | |
| consent process or documentation | | |

^{a} Important protocol deviation which is not potentially impacting efficacy analyses or randomization/drug allocation irregularities
Note:
Percentages are calculated using the number of participants randomized as denominator

2.3. Breaking of the Blind

A code break was performed by the Investigator for 1 participant in the RIPK1 Inhibitor group for safety concerns related to Aes.

2.4. Data Sets Analyzed

The number of participants included in each analysis population is provided in Table 6.

Of note, 1 of the 68 randomized participants did not receive any dose of study treatment due to voluntary withdrawal, and was not included in the analysis population.

TABLE 6
Analysis population

| | | RIPK1 Inhibitor | Not | |
|---|---|---|---|---|
| | Placebo | 600 mg | randomized | All |
| Enrolled population | 21 | 47 | 14 | 82 |
| Randomized population | 21 | 47 | 0 | 68 |
| Safety population | 20 | 47 | 0 | 67 |
| Efficacy population | 19 | 41 | 0 | 60 |
| Pharmacokinetic | 0 | 47 | 0 | 47 |
| population | | | | |

Note:
The efficacy, safety and pharmacokinetic population participants are tabulated according to treatment actually received (as treated). For the other populations, participants are tabulated according to the treatment group allocated by IVRS/IWRS (as randomized).

2.5. Demographic and Other Baseline Characteristics

2.5.1. Demography

Demography and participant characteristics at baseline were generally balanced between the two treatment groups, with the exception of the percentage of participants with BMI≥40 kg/m² (who are subjected to a higher risk of acute respiratory distress syndrome), which was greater in the RIPK1 Inhibitor group (n=8; 17.0%) than in the placebo group (n=1; 5.0%). (Table 7).

Overall, 83.6% of participants were White, 7.5% of participants were Black or African American, 4.5% of participants were Unknown, and 3.0% of participants were American Indian or Alaska native; of which 59.7% were male and 40.3% were female, ranging in age between 26 years and 80 years (mean [SD]: 57.8 [12.0]).

TABLE 7
Demographics and participant characteristics
at baseline - Safety population

| | | | RIPK1 Inhibitor | | | |
|---|---|---|---|---|---|---|
| | Placebo | | 600 mg | | All | |
| | (N = 20) | | (N = 47) | | (N = 67) | |
| Age (years) | | | | | | |
| Number | 20 | | 47 | | 67 | |
| Mean (SD) | 55.2 | (13.5) | 58.9 | (11.3) | 57.8 | (12.0) |
| Median | 55.5 | | 60.0 | | 60.0 | |
| Min; Max | 29; 75 | | 26; 80 | | 26; 80 | |
| Sex [n(%)] | | | | | | |
| Number | 20 | | 47 | | 67 | |
| Male | 12 | (60.0) | 28 | (59.6) | 40 | (59.7) |
| Female | 8 | (40.0) | 19 | (40.4) | 27 | (40.3) |
| Race [n(%)] | | | | | | |
| Number | 20 | | 47 | | 67 | |
| White | 16 | (80.0) | 40 | (85.1) | 56 | (83.6) |
| Black or | 2 | (10.0) | 3 | (6.4) | 5 | (7.5) |
| African American | | | | | | |
| American Indian | 1 | (5.0) | 1 | (2.1) | 2 | (3.0) |
| or Alaska Native | | | | | | |
| Unknown | 1 | (5.0) | 2 | (4.3) | 3 | (4.5) |
| Multiple | 0 | | 1 | (2.1) | 1 | (1.5) |
| American Indian | 0 | | 1 | (2.1) | 1 | (1.5) |
| or Alaska | | | | | | |
| Native/White | | | | | | |
| Ethnicity | | | | | | |
| [n (%)] | | | | | | |
| Number | 20 | | 47 | | 67 | |
| Hispanic | 13 | (65.0) | 30 | (63.8) | 43 | (64.2) |
| or Latino | | | | | | |
| Not Hispanic | 6 | (30.0) | 14 | (29.8) | 20 | (29.9) |
| or Latino | | | | | | |
| Not Reported | 1 | (5.0) | 1 | (2.1) | 2 | (3.0) |
| Unknown | 0 | | 2 | (4.3) | 2 | (3.0) |
| Baseline | | | | | | |
| Weight (kg) | | | | | | |
| Number | 20 | | 47 | | 67 | |
| Mean (SD) | 88.9 | (19.3) | 89.1 | (19.7) | 89.1 | (19.4) |
| Median | 86.0 | | 85.2 | | 86.0 | |
| Min; Max | 62; 148 | | 55; 150 | | 55; 150 | |
| BMI (kg/m²) | | | | | | |
| [n(%)] | | | | | | |
| Number | 20 | | 47 | | 67 | |
| 18.5-<25 | 2 | (10.0) | 6 | (12.8) | 8 | (11.9) |
| 25-<30 | 8 | (40.0) | 20 | (42.6) | 28 | (41.8) |
| 30-<40 | 9 | (45.0) | 13 | (27.7) | 22 | (32.8) |
| ≥40 | 1 | (5.0) | 8 | (17.0) | 9 | (13.4) |

BMI: Body mass index

2.5.2. Medical History

The medical history profiles specific for this study were balanced between treatment arms (Table 8).

TABLE 8
Medical history - Specific medical history - Safety population

| | | RIPK1 Inhibitor | | | |
|---|---|---|---|---|---|
| Medical History | Placebo | 600 mg | | All | |
| Group n (%) | (N = 20) | (N = 47) | | (N = 67) | |
| Obesity | 10 (50.0) | 22 | (46.8) | 32 | (47.8) |
| Diabetes | 4 (20.0) | 17 | (36.2) | 21 | (31.3) |
| Respiratory | 4 (20.0) | 8 | (17.0) | 12 | (17.9) |
| Disorders | | | | | |
| Renal Disorders | 1 (5.0) | 7 | (14.9) | 8 | (11.9) |
| Cardiovascular | 2 (10.0) | 4 | (8.5) | 6 | (9.0) |
| Disorders | | | | | |
| Autoimmune | 2 (10.0) | 1 | (2.1) | 3 | (4.5) |
| Disorders | | | | | |

n (%) = number and percentage of participants with at least one medical history
Note:
A participant can be counted in several categories, but not more than once within a given category. Groups are sorted by decreasing frequency in the overall treatment group Cardiovascular category corresponds to any participant with a medical history event in the Cardiac Disorder System Organ Class (SOC).
Diabetes category corresponds to any participant reporting medical history of Type 1 or Type 2 Diabetes.
Obesity category corresponds to any participant with baseline BMI ≥30 kg/m² or reporting medical history of obesity.
Renal category corresponds to any participant with a medical history event in the Renal and Urinary Disorder SOC.
Respiratory category corresponds to any participant with a medical history event in the Respiratory, Thoracic and Mediastinal Disorder SOC.
Autoimmune disorders category is based on autoimmune disorders identified from the blinded review of the medical history listing: i.e., autoimmune thyroiditis, immune thrombocytopenia and, rheumatoid arthritis.

2.5.3. Disease Characteristics at Baseline

Participants disease characteristics at baseline were generally balanced across treatment arms (Table 9, Table 10).

Mean baseline CRP (mg/L) values were of 113.9 and the range across groups was 10 to 425. The mean baseline CRP (mg/L) for the placebo and RIPK1 Inhibitor groups are 133.5 (median=110.2) and 105.6 (median=89.1), respectively. While baseline CRP level was higher in the placebo group than in the RIPK1 Inhibitor group, COVID-19 severity at study entry was comparable overall among participants of the two treatment groups.

Mean days since COVID-19 diagnosis values were 7.8 days and the range across groups was 1 day to 20 days. Mean days since COVID-19 hospitalization values were 2.9 days and the range across groups was 0 day to 13 days.

Mean baseline SpO₂/FiO₂ (ratio) value was 296.0 and the range across groups was 120 to 457.

TABLE 9
Disease characteristics at baseline - Safety population

| | | | RIPK1 Inhibitor | | | |
|---|---|---|---|---|---|---|
| | Placebo | | 600 mg | | All | |
| | (N = 20) | | (N = 47) | | (N = 67) | |
| Days since | | | | | | |
| COVID-19 | | | | | | |
| diagnosis | | | | | | |
| Number | 20 | | 47 | | 67 | |
| Mean (SD) | 7.7 | (3.7) | 7.8 | (5.1) | 7.8 | (4.7) |
| Median | 8.5 | | 8.0 | | 8.0 | |
| Min; Max | 2; 14 | | 1; 20 | | 1; 20 | |
| Days since | | | | | | |
| COVID-19 | | | | | | |
| hospitalization | | | | | | |
| Number | 20 | | 47 | | 67 | |
| Mean (SD) | 3.1 | (2.8) | 2.8 | (1.5) | 2.9 | (2.0) |
| Median | 3.0 | | 2.0 | | 2.0 | |
| Min; Max | 0; 13 | | 1; 7 | | 0; 13 | |
| ICU admission | | | | | | |
| at Baseline | | | | | | |
| Number | 20 | | 47 | | 67 | |
| No | 17 | (85.0) | 46 | (97.9) | 63 | (94.0) |
| Yes | 3 | (15.0) | 1 | (2.1) | 4 | (6.0) |
| Baseline Mean | | | | | | |
| Arterial | | | | | | |
| Pressure | | | | | | |
| (mmHg) | | | | | | |
| Number | 20 | | 47 | | 67 | |
| Mean (SD) | 93.5 | (12.3) | 93.0 | (9.1) | 93.2 | (10.1) |
| Median | 94.8 | | 93.7 | | 93.7 | |
| Min; Max | 73; 112 | | 75; 123 | | 73; 123 | |
| Baseline | | | | | | |
| CRP (mg/L) | | | | | | |
| Number | 20 | | 47 | | 67 | |
| Mean (SD) | 133.5 | (88.4) | 105.6 | (67.2) | 113.9 | (74.6) |
| Median | 110.2 | | 89.1 | | 93.0 | |
| Min; Max | 53; 425 | | 10; 303 | | 10; 425 | |
| Baseline | | | | | | |
| SpO₂/ | | | | | | |
| FiO₂ (RATIO) | | | | | | |
| Number | 20 | | 47 | | 67 | |
| Mean (SD) | 294.1 | (55.3) | 296.8 | (62.9) | 296.0 | (60.3) |
| Median | 299.1 | | 293.9 | | 293.9 | |
| Min; Max | 141; 380 | | 120; 457 | | 120; 457 | |
| Oxygen Therapy | | | | | | |
| at Baseline | | | | | | |
| Number | 20 | | 47 | | 67 | |
| Nasal Cannula | 13 | (65.0) | 25 | (53.2) | 38 | (56.7) |
| Simple Face Mask | 6 | (30.0) | 17 | (36.2) | 23 | (34.3) |
| Non-Rebreather | 0 | | 1 | (2.1) | 1 | (1.5) |
| Face Mask | | | | | | |
| High-Flow Nasal | 1 | (5.0) | 1 | (2.1) | 2 | (3.0) |
| Cannula | | | | | | |
| Non-Invasive | 0 | | 1 | (2.1) | 1 | (1.5) |
| Ventilation | | | | | | |
| Ambient | 0 | | 2 | (4.3) | 2 | (3.0) |

ICU: Intensive Care Unit,
SpO₂/FiO₂: Peripheral oxygen saturation/Fraction of inspired oxygen,
CRP: C-Reactive Protein
Note:
Baseline is defined as the last available and evaluable value before the first administration of the Investigational Medicinal Product.

TABLE 10
Disease characteristics at baseline - Efficacy population

| | | | RIPK1 Inhibitor | | | |
|---|---|---|---|---|---|---|
| | Placebo | | 600 mg | | All | |
| | (N = 19) | | (N = 41) | | (N = 60) | |
| Days since | | | | | | |
| COVID-19 | | | | | | |
| diagnosis | | | | | | |
| Number | 19 | | 41 | | 60 | |
| Mean (SD) | 7.7 | (3.8) | 8.0 | (4.9) | 7.9 | (4.5) |
| Median | 9.0 | | 8.0 | | 8.0 | |
| Min; Max | 2; 14 | | 1; 20 | | 1; 20 | |
| Days since | | | | | | |
| COVID-19 | | | | | | |
| hospitalization | | | | | | |
| Number | 19 | | 41 | | 60 | |
| Mean (SD) | 3.2 | (2.9) | 2.9 | (1.6) | 3.0 | (2.1) |
| Median | 3.0 | | 2.0 | | 2.0 | |
| Min; Max | 0; 13 | | 1; 7 | | 0; 13 | |
| ICU admission | | | | | | |
| at Baseline | | | | | | |
| Number | 19 | | 41 | | 60 | |
| No | 16 | (84.2) | 41 | (100) | 57 | (95.0) |
| Yes | 3 | (15.8) | 0 | | 3 | (5.0) |
| Baseline Mean | | | | | | |
| Arterial | | | | | | |
| Pressure | | | | | | |
| (mmHg) | | | | | | |
| Number | 19 | | 41 | | 60 | |
| Mean (SD) | 93.8 | (12.6) | 93.4 | (9.4) | 93.5 | (10.4) |
| Median | 99.3 | | 94.0 | | 94.3 | |
| Min; Max | 73; 112 | | 75; 123 | | 73; 123 | |
| Baseline | | | | | | |
| CRP (mg/L) | | | | | | |
| Number | 19 | | 41 | | 60 | |
| Mean (SD) | 137.5 | (88.9) | 114.8 | (66.2) | 122.0 | (74.1) |
| Median | 111.4 | | 93.0 | | 99.4 | |
| Min; Max | 53; 425 | | 48; 303 | | 48; 425 | |
| Baseline | | | | | | |
| SpO₂/ | | | | | | |
| FiO₂ (RATIO) | | | | | | |
| Number | 19 | | 41 | | 60 | |
| Mean (SD) | 292.5 | (56.3) | 298.0 | (58.0) | 296.3 | (57.1) |
| Median | 287.9 | | 306.3 | | 300.1 | |
| Min; Max | 141; 380 | | 120; 457 | | 120; 457 | |
| Oxygen Therapy | | | | | | |
| at Baseline | | | | | | |
| Number | 19 | | 41 | | 60 | |
| Nasal Cannula | 13 | (68.4) | 23 | (56.1) | 36 | (60.0) |
| Simple Face Mask | 5 | (26.3) | 14 | (34.1) | 19 | (31.7) |
| Non-Rebreather | 0 | | 1 | (2.4) | 1 | (1.7) |
| Face Mask | | | | | | |
| High-Flow Nasal | 1 | (5.3) | 1 | (2.4) | 2 | (3.3) |
| Cannula | | | | | | |

| | | | | | |
|---|---|---|---|---|---|
| Non-Invasive | 0 | 1 | (2.4) | 1 | (1.7) |
| Ventilation | | | | | |
| Ambient | 0 | 1 | (2.4) | 1 | (1.7) |

ICU: Intensive Care Unit,
SpO₂/FiO₂: Peripheral oxygen saturation/Fraction of inspired oxygen,
CRP: C-Reactive Protein
Note:
Baseline is defined as the last available and evaluable value before the first administration of the Investigational Medicinal Product.

2.5.4. Prior and/or Concomitant Medication

Prior Medication

The use of specified major classes of prior medications are largely balanced between treatment groups. The most frequently used concomitant medications by medication name were dexamethasone and azithromycin for both treatment groups, both medications were taken by more than 5 participants in each group. Corticosteroids as standard of care were administered in approximately 65% of the participants (65.0% in the placebo group; 63.8% in the RIPK1 Inhibitor group) in each treatment group (Table 11).

TABLE 11
Prior medications - Specific medications - safety population

| | | | RIPK1 Inhibitor | | | |
|---|---|---|---|---|---|---|
| Medication | Placebo | | 600 mg | | All | |
| Group n (%) | (N = 20) | | (N = 47) | | (N = 67) | |
| ACEI/ARB | 8 | (40.0) | 16 | (34.0) | 24 | (35.8) |
| Losartan | 3 | (15.0) | 5 | (10.6) | 8 | (11.9) |
| Enalapril | 2 | (10.0) | 4 | (8.5) | 6 | (9.0) |
| Valsartan | 2 | (10.0) | 1 | (2.1) | 3 | (4.5) |
| Captopril | 0 | | 2 | (4.3) | 2 | (3.0) |
| Enalapril Maleate | 1 | (5.0) | 1 | (2.1) | 2 | (3.0) |
| Lisinopril | 0 | | 2 | (4.3) | 2 | (3.0) |
| Losartan Potassium | 0 | | 2 | (4.3) | 2 | (3.0) |
| Hydrochlorothiazide; | 0 | | 1 | (2.1) | 1 | (1.5) |
| losartan | | | | | | |
| Antimicrobials | 9 | (45.0) | 23 | (48.9) | 32 | (47.8) |
| Azithromycin | 6 | (30.0) | 18 | (38.3) | 24 | (35.8) |
| Oseltamivir | 1 | (5.0) | 3 | (6.4) | 4 | (6.0) |
| Clarithromycin | 1 | (5.0) | 2 | (4.3) | 3 | (4.5) |
| Favipiravir | 0 | | 3 | (6.4) | 3 | (4.5) |
| Hydroxychloroquine | 1 | (5.0) | 2 | (4.3) | 3 | (4.5) |
| Sulfate | | | | | | |
| Oseltamivir | 2 | (10.0) | 1 | (2.1) | 3 | (4.5) |
| Phosphate | | | | | | |
| Ivermectin | 0 | | 2 | (4.3) | 2 | (3.0) |
| Steroid treatment | 13 | (65.0) | 30 | (63.8) | 43 | (64.2) |
| Dexamethasone | 13 | (65.0) | 29 | (61.7) | 42 | (62.7) |
| Prednisone | 0 | | 4 | (8.5) | 4 | (6.0) |
| Methylprednisolone | 1 | (5.0) | 1 | (2.1) | 2 | (3.0) |

IMP: Investigational medicinal product
n (%) = number and percentage of participants with at least one prior medication
Prior medications are those the participant used before the day of the first IMP intake. Prior medications can be discontinued before first IMP administration or can be ongoing during treatment phase.

Concomitant Medications

All participants used at least one concomitant medication during the study period. The use of selected classes of concomitant medications are balanced between treatment groups, particularly in the antimicrobial and steroid treatment (Table 12).

There were 2 (10.0%) participants in the placebo group and 4 (8.5%) participants in the RIPK1 Inhibitor group, who received IL-6 blocker tocilizumab as a concomitant medication.

The summary of post-treatment medications for the same subset of medications are provided in Table 13.

TABLE 12
Concomitant medications - Specific medications - safety population

| | | |
|---|---|---|
| | | RIPK1 Inhibitor |
| Medication | Placebo | 600 mg |
| Group n (%) | (N = 20) | (N = 47) |

| | | | | |
|---|---|---|---|---|
| ACEI/ARB | 7 | (35.0) | 22 | (46.8) |
| Losartan | 2 | (10.0) | 10 | (21.3) |
| Enalapril | 2 | (10.0) | 4 | (8.5) |

| | | | |
|---|---|---|---|
| Captopril | 0 | 3 | (6.4) |

| | | | | |
|---|---|---|---|---|
| Lisinopril | 1 | (5.0) | 2 | (4.3) |

| | | | |
|---|---|---|---|
| Losartan Potassium | 0 | 2 | (4.3) |

| | | | | |
|---|---|---|---|---|
| Valsartan | 2 | (10.0) | 2 | (4.3) |
| Enalapril Maleate | 1 | (5.0) | 1 | (2.1) |
| Antimicrobials | 9 | (45.0) | 22 | (46.8) |
| Azithromycin | 7 | (35.0) | 16 | (34.0) |

| | | | |
|---|---|---|---|
| Favipiravir | 0 | 4 | (8.5) |

| Clarithromycin | 1 | (5.0) | 2 | (4.3) |
|---|---|---|---|---|
| Hydroxychloroquine Sulfate | 2 | (10.0) | 2 | (4.3) |
| Oseltamivir | 1 | (5.0) | 0 | |
| Oseltamivir Phosphate | 2 | (10.0) | 0 | |
| IL-6 Blocker | 2 | (10.0) | 4 | (8.5) |
| Tocilizumab | 2 | (10.0) | 4 | (8.5) |
| Steroid treatment | 19 | (95.0) | 38 | (80.9) |
| Dexamethasone | 19 | (95.0) | 36 | (76.6) |
| Hydrocortisone | 2 | (10.0) | 1 | (2.1) |
| Methylprednisolone | 1 | (5.0) | 1 | (2.1) |

| | | | |
|---|---|---|---|
| Prednisone | 0 | 1 | (2.1) |

IMP: Investigational medicinal product,
TEAE: Treatment emergent adverse event
n (%) = number and percentage of participants with at least one concomitant medication Concomitant medications are any treatments received by the participant during the TEAE period (from first IMP intake up to and including the day of last dose of study intervention plus 5 days)

TABLE 13
Post-treatment medications - Specific
medications - safety population

| | | |
|---|---|---|
| | | RIPK1 Inhibitor |
| Medication | Placebo | 600 mg |
| Group n (%) | (N = 20) | (N = 47) |

| | | | |
|---|---|---|---|
| ACEI/ARB | 6 | (30.0) | 15 (31.9) |
| Losartan | 2 | (10.0) | 6 (12.8) |
| Enalapril | 1 | (5.0) | 3 (6.4) |

| | | |
|---|---|---|
| Losartan Potassium | 0 | 2 (4.3) |

| | | | |
|---|---|---|---|
| Valsartan | 2 | (10.0) | 2 (4.3) |

| | | |
|---|---|---|
| Captopril | 0 | 1 (2.1) |

| | | | |
|---|---|---|---|
| Enalapril Maleate | 1 | (5.0) | 1 (2.1) |

| | | |
|---|---|---|
| Lisinopril | 0 | 1 (2.1) |

| | | | |
|---|---|---|---|
| Antimicrobials | 1 | (5.0) | 1 (2.1) |

| | | |
|---|---|---|
| Favipiravir | 0 | 1 (2.1) |

| | | | |
|---|---|---|---|
| Azithromycin | 1 | (5.0) | 0 |
| Steroid treatment | 4 | (20.0) | 2 (4.3) |
| Dexamethasone | 3 | (15.0) | 2 (4.3) |

| | | |
|---|---|---|
| Hydrocortisone | 0 | 1 (2.1) |
| Prednisone | 0 | 1 (2.1) |

| | | | |
|---|---|---|---|
| Methylprednisolone | 1 | (5.0) | 0 |

IMP: Investigational medicinal product, TEAE: Treatment emergent adverse event
n (%) = number and percentage of participants with at least one post-treatment medication Post-treatment medications are those the participant took after the TEAE period (from first IMP intake up to and including the day of last dose of study intervention plus 5 days)

### 3. EFFICACY/PHARMACODYNAMICS EVALUATION

3.1. Primary Pharmacodynamics Endpoint

3.1.1. Primary Analysis

Relative Change from Baseline in CRP Level on Day 7

At Day 7, the observed mean (SD; n) of CRP decreased from 114.8 mg/L (66.2; 41) at baseline to 24.2 mg/L (30.6; 20) in the RIPK1 Inhibitor arm, and from 137.5 mg/L (88.9; 19) at baseline to 48.4 mg/L (70.5; 11) in the placebo arm (Table 17). It is noteworthy that at Day 7 only 57.9% (11 of 19 participants) of the data were available in the placebo group and even less in the RIPK1 Inhibitor group: 48.8% (20 of 41 participants). This was mainly linked to participants being discharged from hospital due to COVID-19 recovery before Day 7.

Missing CRP values were imputed with the LOCF approach. When imputing missing CRP values, the observed mean (SD) of CRP at Day 7 was equal to 28.1 mg/L (31.4) in the RIPK1 Inhibitor arm, and to 46.7 mg/L (58.5) in the placebo arm. The mean (SD; median) of relative change from baseline in CRP was numerically lower in the RIPK1 Inhibitor group (0.315 [0.483; 0.165]) as compared to the placebo group (0.490 [0.657; 0.188]). This confirms the larger decrease in CRP values from baseline to Day 7 in RIPK1 Inhibitor group than in the placebo group described below for the primary analysis.

In the primary MMRM analysis, the ratio of the adjusted relative change from baseline in CRP with RIPK1 Inhibitor versus placebo on Day 7 was equal to 0.85 (90% CI: 0.49 to 1.45) (Table 14). This difference did not show a statistically significant larger decrease in CRP from baseline in the RIPK1 Inhibitor group versus placebo group at Day 7 (p-value: 0.302).

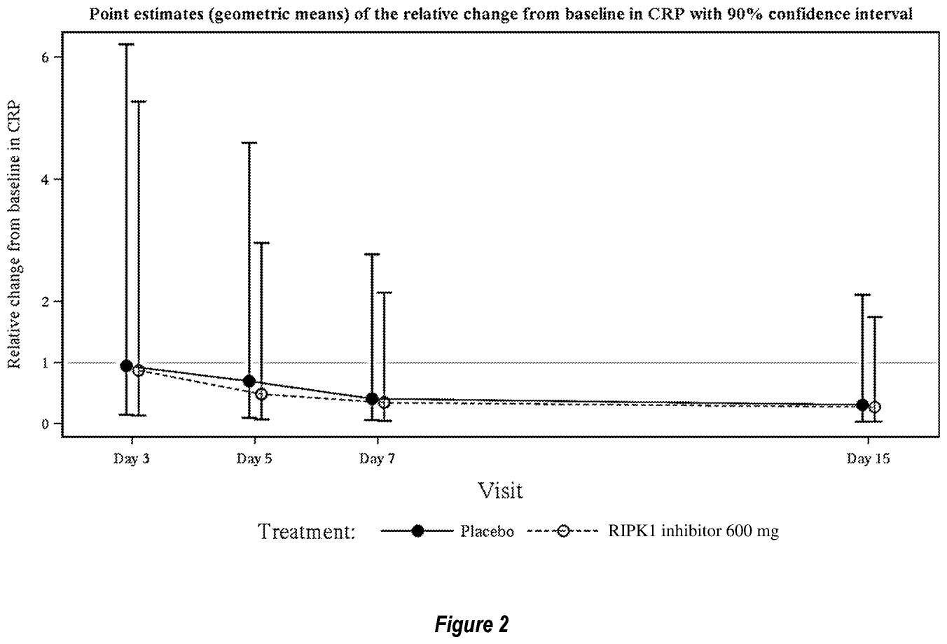

A larger decrease in CRP from baseline in the RIPK1 Inhibitor group versus placebo groups was observed at Day 3, 5, 7 and 15 (Table 15). There was a trend toward a more rapid CRP decrease in the RIPK1 Inhibitor group versus placebo group, as reflected in the adjusted relative change in CRP from baseline on Day 3, Day 5, Day 7 and Day 15 (FIG. 2, Table 15, Table 16).

The treatment difference in relative change from baseline in CRP values with and without imputation of missing data showed little difference before Day 7:
At Day 3, the RIPK1 Inhibitor versus placebo ratios (% CI) were 0.91 (0.63 to 1.32) and 0.92 (0.63 to 1.33) with and without imputation of missing data, respectively,
At Day 5, the RIPK1 Inhibitor versus placebo ratios (% CI) were 0.70 (0.44 to 1.10) and 0.73 (0.42 to 1.25) with and without imputation of missing data, respectively.

Regardless of whether imputation of missing data was used, the largest difference in relative change in CRP level between RIPK1 Inhibitor and placebo arms was observed at Day 5, where the point estimate of relative CRP change from baseline was 0.42 (90% CI: 0.08 to 2.96) for RIPK1 Inhibitor arm and 0.70 (90% CI: 0.11 to 4.60) for the placebo arm (Table 15).

TABLE 14
CRP - Point estimates of the treatment difference between
RIPK1 Inhibitor and placebo at Day 7 in relative change
from baseline with two-sided 90% confidence interval
and one-sided p-value - Efficacy population

| | | Point | 90% | | |
|---|---|---|---|---|---|
| Parameter | Comparison | estimate | CI DF | t-statistic | p-value |
| Relative | RIPK1 | 0.85 | (0.49 to 53.8 | −0.52 | 0.302 |
| change from | Inhibitor vs | | 1.45) | | |
| baseline | placebo | | | | |
| in CRP | at Day 7 | | | | |

The linear mixed effects model on log (relative change in CRP) includes baseline log-CRP, visit, treatment group and visit-by-treatment group interaction as fixed effects and sites as a random effect. Repeated measures within participants are modeled with an unstructured residual covariance matrix. Point estimate obtained is back-transformed by exponentiation (point estimate displayed).
Point estimate: a value lower than 1 indicates a larger decrease from baseline in treatment group than in placebo group.
Null hypothesis: decrease from baseline (log-relative change from baseline) is equal or larger in placebo group than in treatment group; null hypothesis is rejected if p-value is lower than 0.05.
Missing values for the relative change from baseline in CRP for Days 3, 5, 7, 15 were replaced following the LOCF approach. When several values are available on a day, the last available and evaluable value is considered for the analysis.

TABLE 15
CRP - Point estimates of the relative change from
baseline (geometric means) with two-sided 90%
confidence interval - Efficacy population

| | | Point | |
|---|---|---|---|
| Parameter | Label | estimate | 90% CI |
| Relative | Placebo at Day 03 | 0.96 | (0.15 to 6.21) |
| change from | Placebo at Day 05 | 0.70 | (0.11 to 4.60) |
| baseline | Placebo at Day 07 | 0.42 | (0.06 to 2.77) |
| in CRP | Placebo at Day 15 | 0.31 | (0.05 to 2.12) |
| | RIPK1 Inhibitor at Day 03 | 0.87 | (0.14 to 5.28) |
| | RIPK1 Inhibitor at Day 05 | 0.49 | (0.08 to 2.96) |
| | RIPK1 Inhibitor at Day 07 | 0.35 | (0.06 to 2.16) |
| | RIPK1 Inhibitor at Day 15 | 0.28 | (0.05 to 1.75) |

The linear mixed effects model on log (relative change in CRP) includes baseline log-CRP, visit, treatment group and visit-by-treatment group interaction as fixed effects and sites as a random effect. Repeated measures within participants are modeled with an unstructured residual covariance matrix. Point estimate obtained is back-transformed to original scale by exponentiation (point estimate displayed).
Point estimate: a value lower than 1 indicates a decrease from baseline.
Missing values for the relative change from baseline in CRP for Days 3, 5, 7, 15 were replaced following the LOCF approach. When several values are available on a day, the last available and evaluable value is considered for the analysis.

TABLE 16
CRP - Point estimates of the relative change from baseline
(geometric means) with two-sided 90% confidence interval
displayed as percent change - Efficacy population

| | | Point | |
|---|---|---|---|
| Parameter | Label | estimate | 90% CI |
| Percent | Placebo at Day 03 | −4.44 | (−85.30 to 521.37) |
| change from | Placebo at Day 05 | −30.20 | (−89.41 to 359.84) |
| baseline | Placebo at Day 07 | −58.41 | (−93.77 to 177.42) |
| in CRP | Placebo at Day 15 | −68.93 | (−95.44 to 111.66) |
| | RIPK1 Inhibitor at Day 03 | −12.85 | (−85.61 to 427.67) |
| | RIPK1 Inhibitor at Day 05 | −51.43 | (−92.03 to 195.92) |
| | RIPK1 Inhibitor at Day 07 | −64.81 | (−94.26 to 115.73) |
| | RIPK1 Inhibitor at Day 15 | −71.76 | (−95.44 to 74.89) |

The linear mixed effects model on log (relative change in CRP) includes baseline log-CRP, visit, treatment group and visit-by-treatment group interaction as fixed effects and sites as a random effect. Repeated measures within participants are modeled with an unstructured residual covariance matrix. The percent change (point estimate displayed) is obtained by subtracting 1 from the antilog transformation of the point estimate and multiplying it by 100. Point estimate: a negative value indicates a decrease from baseline.
Missing values for the relative change from baseline in CRP for Days 3, 5, 7, 15 were replaced following the LOCF approach. When several values are available on a day, the last available and evaluable value is considered for the analysis.

3.1.2. Secondary Analyses

An ad hoc sensitivity analysis was performed for the primary analysis for the primary endpoint, with two participants excluded from the analysis population that exhibited unexpected PK data. For these two participants (the first one randomized in the RIPK1 Inhibitor group and the second one randomized in the placebo group) included in the same site on the same day, there was a suspicion of treatment inversion. However, the results of this sensitivity analysis were consistent with the primary analysis.

3.2. Secondary Efficacy/Pharmacodynamics Endpoints

3.2.1. Main Secondary endpoints

3.2.1.1. Time to 50% Decrease from Baseline in CRP Level

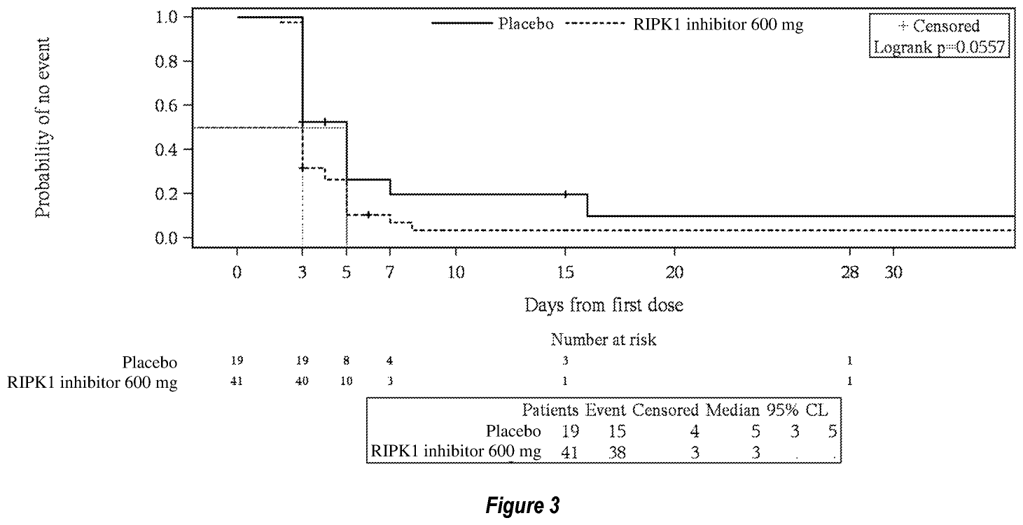

Kaplan-Meier curves for time to 50% improvement in CRP for both treatment arms are provided in FIG. 3. The median time for 50% decrease in CRP level relative to baseline was 3 days for the RIPK1 Inhibitor group, and 5 days for the placebo group.

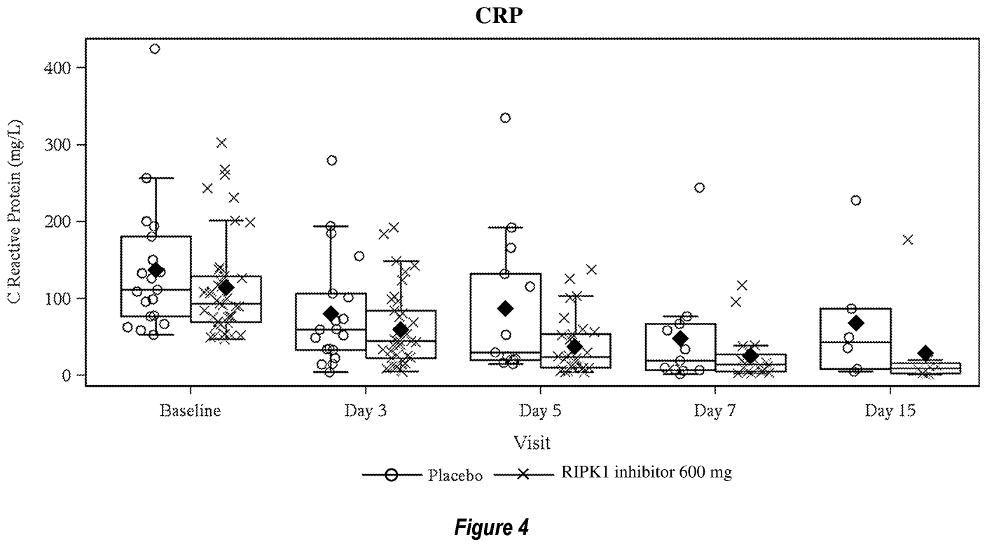

A 50% decrease from baseline in CRP occurred early in the study treatment period for most participants. In the RIPK1 Inhibitor group, 69.2% of participants experienced this event by Day 3 (i.e., while they were still hospitalized), versus 48.4% in placebo group. In the placebo group, the majority of participants (61.5%) achieved 50% decrease from baseline in CRP by Day 5. This trend was confirmed with the raw CRP values (without imputation) with mean relative changes from baseline on Day 3 and Day 5 of 0.75 and 0.69 for placebo, versus 0.58 and 0.37 for RIPK1 Inhibitor, respectively (FIG. 4, Table 17).

A trend toward a more rapid decrease in CRP was observed in the RIPK1 Inhibitor group, where the exploratory p-value (0.0557) of the analyses of the slopes of KR curves demonstrated that the difference between active treatment and placebo groups was very close to statistical significance (FIG. 3).

TABLE 17
CRP - Summary of CRP [mg/L]: raw value and relative change from baseline - Efficacy population

| Raw data | | | | | | | Relative change from baseline | | | | | | |
|---|---|---|---|---|---|---|---|---|---|---|---|---|---|
| N | Mean | SD | SEM | Median | Min | Max | N | Mean | SD | SEM | Median | Min | Max |

Placebo

| | | | | | | | | | | | | | | |
|---|---|---|---|---|---|---|---|---|---|---|---|---|---|---|
| Baseline | 19 | 137.5 | 88.9 | 20.38 | 111.4 | 53 | 425 | | | | | | | |
| Day 3 | 19 | 81.4 | 73.6 | 16.88 | 59.8 | 4 | 280 | 19 | 0.754 | 1.036 | 0.2377 | 0.507 | 0.06 | 4.75 |
| Day 5 | 13 | 87.9 | 97.1 | 26.92 | 30.0 | 15 | 335 | 13 | 0.692 | 0.762 | 0.2113 | 0.198 | 0.06 | 2.24 |
| Day 7 | 11 | 48.4 | 70.5 | 21.25 | 19.4 | 2 | 244 | 11 | 0.420 | 0.625 | 0.1883 | 0.074 | 0.02 | 1.94 |
| Day 15 | 6 | 69.0 | 83.5 | 34.10 | 42.8 | 5 | 228 | 6 | 0.553 | 0.667 | 0.2724 | 0.427 | 0.02 | 1.81 |

RIPK1 Inhibitor 600 mg

| | | | | | | | | | | | | | | |
|---|---|---|---|---|---|---|---|---|---|---|---|---|---|---|
| Baseline | 41 | 114.8 | 66.2 | 10.34 | 93.0 | 48 | 303 | | | | | | | |
| Day 3 | 39 | 59.4 | 49.6 | 7.94 | 44.4 | 5 | 192 | 39 | 0.581 | 0.589 | 0.0942 | 0.384 | 0.07 | 3.01 |
| Day 5 | 31 | 37.7 | 36.8 | 6.62 | 24.2 | 4 | 138 | 31 | 0.368 | 0.511 | 0.0918 | 0.195 | 0.04 | 2.65 |
| Day 7 | 20 | 24.2 | 30.6 | 6.85 | 14.0 | 3 | 118 | 20 | 0.289 | 0.537 | 0.1201 | 0.119 | 0.02 | 2.48 |
| Day 15 | 8 | 29.2 | 59.9 | 21.17 | 9.6 | 1 | 177 | 8 | 0.380 | 0.872 | 0.3082 | 0.092 | 0.01 | 2.53 |

Note:
Baseline is defined as the last available and evaluable value before the first administration of the Investigational Medicinal Product.
Samples were tested at the local laboratory per local practice.

3.2.1.2. Time to Improvement of Oxygenation as Measured by Oxygen Saturation≥92% Breathing Room Air Over 48 Hours or Until Discharge

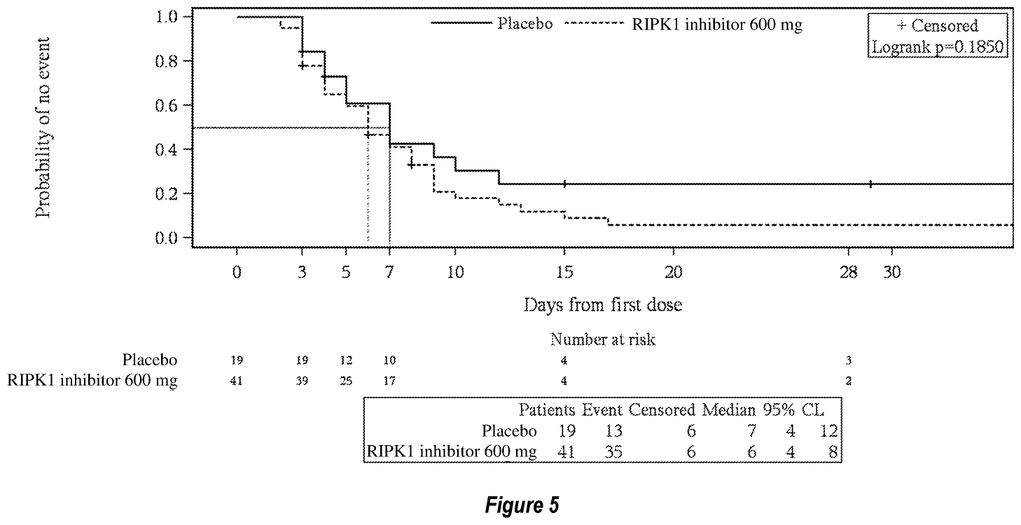

A trend toward a more rapid increase in SpO₂ recovery with RIPK1 Inhibitor was observed in the KM graph with a median of 7 days and 6 days in the placebo and active groups, respectively (FIG. 5). However, there was no statistically significant difference between RIPK1 Inhibitor group and placebo group in the time to improvement of oxygenation, the exploratory p-value on the difference between KM curves was 0.185.

3.2.1.3. Change from Baseline in SpO₂/FiO₂ Ratio at Day 7 (Peripheral Blood Oxygen Saturation/Fraction of Inspired Oxygen)

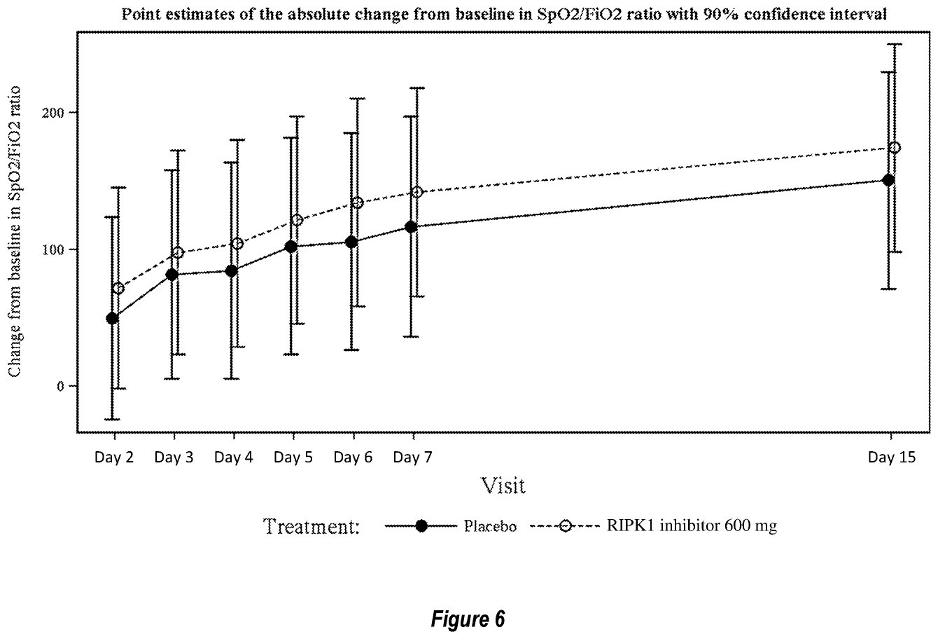

A greater increase (i.e., improvement) was observed in the RIPK1 Inhibitor group versus placebo in the adjusted mean of the change from baseline in SpO₂/FiO₂ ratio at Day 7 with an adjusted treatment difference of 25.24 (90% CI: −21.54 to 72.01) (Table 18). A similar improvement favoring the RIPK1 Inhibitor group over the placebo group was also observed at all visits modelled using a MMRM (i.e., Day 2, 3, 4, 5, 6, 15) with the largest difference observed at Day 6 of 28.71 (90% CI: −15.14 to 72.56) (Table 19, Table 20, FIG. 6).

Mean changes from baseline (SD; median; n) in SpO₂/FiO₂ ratios for placebo and RIPK1 Inhibitor arms in observed data were: −2.5 (58.1; 3.0;19) versus 16.8 (61.2; 3.3; 41) at Day 2; 25 (117.1; 24.1; 16) versus 50.8 (86.5; 47.3; 36) at Day 4; 23.7 (132.2; 45.6; 12) versus 72.5 (89.9; 73.9; 29) at Day 6, 41.2 (149.9; 99.6; 12) versus 89.2 (98.4; 124.1; 21) at Day 7, and in particular 36.1 (190.6; 2.7; 6) versus 160.6 (64.1; 195.1; 8) at Day 15 (Table 20). As a reference, an >20% increase from baseline is considered clinically meaningful (i.e., post baseline increase >60 based on a mean baseline SpO₂/FiO₂ levels around 300 calculated across both groups).

When imputing the missing SpO₂/FiO₂ value with LOCF method, the median changes in SpO₂/FiO₂ ratios from baseline between placebo and RIPK1 Inhibitor arms were 8.3 versus 29.0 at Day 3; 34.3 versus 38.1 at Day 4; 34.3 versus 70.8 at Day 5; 59.4 versus 113.8 at Day 6; 119.2 versus 115.3 at Day 7; 119.2 versus 125.6 at Day 8 and 129.6 versus 135.1 at Day 15. This confirms a trend towards a more rapid improvement in SpO₂/FiO₂ ratio in the RIPK1 Inhibitor group versus placebo group.

TABLE 18
SpO₂/FiO₂ ratio - Point estimates of the treatment
difference between RIPK1 Inhibitor and placebo at
Day 7 in absolute change from baseline with two-sided
90% confidence interval - Efficacy population

| | | Point | |
|---|---|---|---|
| Parameter | Label | estimate | 90% CI |
| Change from | Placebo at Day 07 | 116.97 | (36.66 to 197.29) |
| baseline in | RIPK1 Inhibitor at Day 07 | 142.21 | (65.78 to 218.63) |
| SpO₂/FiO₂ | RIPK1 Inhibitor vs placebo | 25.24 | (−21.54 to 72.01) |
| ratio | at Day 07 | | |

The linear mixed effects model on change in SpO₂/FiO₂ ratio includes baseline value, visit, treatment group and visit-by-treatment group interaction as fixed effects and sites as a random effect. Repeated measures within participants are modeled with an unstructured residual covariance matrix.
Point estimate: a positive value in the difference indicates a larger improvement from baseline in SpO₂/FiO₂ ratio in treatment group than in placebo group.
Missing values were replaced following the LOCF approach. When several values are available on a day, the most severe measurement of the day based on the SpO₂/FiO₂ ratio is considered for the analysis.

TABLE 19
SpO₂/FiO₂ ratio - Point estimates of the absolute change from baseline
with two-sided 90% confidence interval - Efficacy population

| | | Point | |
|---|---|---|---|
| Parameter | Label | estimate | 90% CI |
| Change from | Placebo at Day 02 | 49.91 | (−24.29 to 124.12) |
| baseline in | Placebo at Day 03 | 82.04 | (5.68 to 158.39) |
| SpO₂/FiO₂ | Placebo at Day 04 | 84.91 | (5.95 to 163.87) |
| ratio | Placebo at Day 05 | 102.55 | (23.28 to 181.82) |
| | Placebo at Day 06 | 105.95 | (26.73 to 185.18) |
| | Placebo at Day 07 | 116.97 | (36.66 to 197.29) |
| | Placebo at Day 15 | 150.78 | (71.65 to 229.90) |
| | RIPK1 Inhibitor at Day 02 | 72.02 | (−1.53 to 145.56) |
| | RIPK1 Inhibitor at Day 03 | 97.90 | (23.35 to 172.45) |
| | RIPK1 Inhibitor at Day 04 | 104.61 | (28.83 to 180.39) |
| | RIPK1 Inhibitor at Day 05 | 121.79 | (45.87 to 197.72) |
| | RIPK1 Inhibitor at Day 06 | 134.66 | (58.76 to 210.57) |
| | RIPK1 Inhibitor at Day 07 | 142.21 | (65.78 to 218.63) |
| | RIPK1 Inhibitor at Day 15 | 174.65 | (98.79 to 250.51) |

The linear mixed effects model on change in SpO₂/FiO₂ ratio includes baseline value, visit, treatment group and visit-by-treatment group interaction as fixed effects and sites as a random effect. Repeated measures within participants are modeled with an unstructured residual covariance matrix.
Point estimate: a positive value indicates an improvement from baseline in SpO₂/FiO₂ ratio. Missing values were replaced following the LOCF approach. When several values are available on a day, the most severe measurement of the day based on the SpO₂/FiO₂ ratio is considered for the analysis.

TABLE 20
SpO₂/FiO₂ ratio - Summary of SpO₂/FiO₂ ratio: raw value and change from
baseline - Efficacy population

| Raw data | | | | | | | Change from baseline | | | | | | |
|---|---|---|---|---|---|---|---|---|---|---|---|---|---|
| N | Mean | SD | SEM | Median | Min | Max | N | Mean | SD | SEM | Median | Min | Max |

Placebo

| | | | | | | | | | | | | | | |
|---|---|---|---|---|---|---|---|---|---|---|---|---|---|---|
| Baseline | 19 | 292.5 | 56.3 | 12.92 | 287.9 | 141 | 380 | | | | | | | |
| Day 2 | 19 | 290.0 | 80.8 | 18.53 | 287.9 | 96 | 452 | 19 | −2.5 | 58.1 | 13.33 | 3.0 | −109 | 119 |
| Day 3 | 19 | 322.1 | 108.4 | 24.86 | 320.0 | 95 | 462 | 19 | 29.6 | 84.3 | 19.35 | 8.3 | −110 | 172 |
| Day 4 | 16 | 308.5 | 130.6 | 32.65 | 273.8 | 93 | 457 | 16 | 25.0 | 117.1 | 29.27 | 24.1 | −179 | 174 |
| Day 5 | 14 | 314.2 | 130.3 | 34.84 | 295.9 | 90 | 462 | 14 | 40.8 | 127.5 | 34.08 | 3.7 | −163 | 190 |
| Day 6 | 12 | 295.8 | 136.9 | 39.52 | 315.3 | 86 | 462 | 12 | 23.7 | 132.2 | 38.17 | 45.6 | −167 | 190 |
| Day 7 | 12 | 313.3 | 156.0 | 45.03 | 358.3 | 88 | 462 | 12 | 41.2 | 149.9 | 43.27 | 99.6 | −187 | 200 |
| Day 8 | 11 | 309.7 | 158.7 | 47.86 | 368.0 | 91 | 457 | 11 | 37.1 | 144.9 | 43.69 | 119.2 | −166 | 205 |
| Day 9 | 10 | 322.4 | 161.4 | 51.05 | 387.9 | 91 | 457 | 10 | 53.6 | 147.2 | 46.55 | 127.8 | −160 | 205 |
| Day 10 | 9 | 306.3 | 169.7 | 56.55 | 373.1 | 91 | 462 | 9 | 43.3 | 154.0 | 51.32 | 124.0 | −182 | 215 |
| Day 11 | 7 | 279.7 | 168.0 | 63.50 | 326.7 | 80 | 467 | 7 | 23.4 | 169.1 | 63.93 | −5.5 | −173 | 239 |
| Day 12 | 6 | 265.0 | 168.2 | 68.69 | 236.7 | 93 | 457 | 6 | 22.3 | 193.3 | 78.92 | −55.1 | −139 | 316 |
| Day 13 | 6 | 273.1 | 179.9 | 73.44 | 261.4 | 96 | 457 | 6 | 30.3 | 197.1 | 80.46 | −18.3 | −160 | 307 |
| Day 14 | 6 | 299.7 | 160.9 | 65.68 | 320.5 | 116 | 457 | 6 | 57.0 | 184.1 | 75.16 | 52.2 | −147 | 311 |
| Day 15 | 6 | 278.8 | 157.2 | 64.18 | 271.0 | 106 | 462 | 6 | 36.1 | 190.6 | 77.79 | 2.7 | −173 | 307 |

RIPK1 Inhibitor 600 mg

| | | | | | | | | | | | | | | |
|---|---|---|---|---|---|---|---|---|---|---|---|---|---|---|
| Baseline | 41 | 298.0 | 58.0 | 9.06 | 306.3 | 120 | 457 | | | | | | | |
| Day 2 | 41 | 314.8 | 80.5 | 12.57 | 290.9 | 160 | 452 | 41 | 16.8 | 61.2 | 9.55 | 3.3 | −119 | 135 |
| Day 3 | 40 | 337.9 | 85.7 | 13.55 | 330.6 | 154 | 467 | 40 | 43.9 | 74.2 | 11.73 | 30.9 | −103 | 188 |
| Day 4 | 36 | 340.4 | 98.9 | 16.49 | 355.5 | 93 | 462 | 36 | 50.8 | 86.5 | 14.41 | 47.3 | −114 | 206 |
| Day 5 | 32 | 350.5 | 107.3 | 18.97 | 356.1 | 96 | 462 | 32 | 62.7 | 92.1 | 16.27 | 69.6 | −133 | 198 |
| Day 6 | 29 | 358.7 | 111.3 | 20.67 | 384.0 | 86 | 467 | 29 | 72.5 | 89.9 | 16.70 | 73.9 | −142 | 202 |
| Day 7 | 21 | 367.1 | 101.3 | 22.10 | 418.2 | 97 | 462 | 21 | 89.2 | 98.4 | 21.47 | 124.1 | −123 | 206 |
| Day 8 | 18 | 371.5 | 107.6 | 25.37 | 433.3 | 91 | 476 | 18 | 99.2 | 106.6 | 25.12 | 131.4 | −114 | 225 |
| Day 9 | 16 | 381.8 | 116.6 | 29.16 | 445.2 | 91 | 476 | 16 | 111.2 | 111.6 | 27.90 | 150.0 | −119 | 225 |
| Day 10 | 13 | 386.7 | 112.1 | 31.09 | 447.6 | 90 | 462 | 13 | 119.8 | 107.0 | 29.67 | 133.8 | −110 | 206 |
| Day 11 | 12 | 373.2 | 113.4 | 32.73 | 445.2 | 95 | 462 | 12 | 111.0 | 104.5 | 30.16 | 153.7 | −105 | 204 |
| Day 12 | 12 | 401.0 | 113.2 | 32.68 | 452.4 | 92 | 462 | 12 | 138.8 | 111.7 | 32.25 | 190.3 | −108 | 252 |
| Day 13 | 10 | 389.5 | 125.1 | 39.57 | 457.1 | 94 | 467 | 10 | 129.1 | 123.6 | 39.07 | 195.1 | −106 | 257 |
| Day 14 | 8 | 414.8 | 79.5 | 28.12 | 454.8 | 253 | 462 | 8 | 139.4 | 96.7 | 34.20 | 192.7 | −40 | 209 |
| Day 15 | 8 | 436.0 | 60.2 | 21.29 | 457.1 | 288 | 467 | 8 | 160.6 | 64.1 | 22.67 | 195.1 | 52 | 211 |

Note:
Baseline is defined as the last available and evaluable value before the first administration of the Investigational Medicinal Product.

3.2.1.4. Number of Days without Need for Oxygen Support and Alive (Oxygen Saturation≥92% Breathing Room Air) and Numbers of Ventilator-Free Days (VFD) and of Respiratory Failure-Free Days (RFFD) and Alive Up to Day 28

There was a general trend favoring the RIPK1 Inhibitor treatment group over the placebo group in the observed mean (SD) number of days without need of oxygen support (placebo: 18.0 [10.2]; RIPK1 Inhibitor 600 mg: 20.5 [7.7]), and similarly for mean VFD (SD) (placebo: 23.4 [10.0]; RIPK1 Inhibitor 600 mg: 26.0 [7.4]) and mean RFFD (SD) (placebo: 23.3 [10.0]; RIPK1 Inhibitor 600 mg: 25.9 [7.4]) (Table 21). When not considering the 4 participants who died during the study in the analysis, the difference was less prominent, but still favoring the RIPK1 Inhibitor treatment group.

The selected analysis population was participants who did not require mechanical or high flow oxygen ventilation at study entry. Hence, the maximum number of VFD or RFFD was theoretically 28 days over the study period. Based on the mean values, there was a difference of 3 VFDs or RFFDs between the 2 treatment arms in favor of RIPK1 Inhibitor over the 28-day study period. As a reference, a difference of 2 days between active and placebo in RFFD may be considered as clinically relevant.

An exploratory analysis on the number of days without need for oxygen support and alive, VFDs and alive, and RFFDs and alive up to 15-day treatment period (the theoretically maximum number was 15 days) was performed. A difference of 1 day was observed in the mean days (SD) without need for oxygen support (placebo: 7.8 [5.3], RIPK1 Inhibitor 600 mg: 8.8 [4.6]), VFDs (placebo: 12.4 [5.3], RIPK1 Inhibitor 600 mg: 13.9 [4.0]), and RFFDs (placebo: 12.8 [5.4], RIPK1 Inhibitor 600 mg: 13.9 [4.0]) was observed in favor of RIPK1 Inhibitor group.

TABLE 21
Supplemental oxygen support - Summary of number of days without
need for oxygen support and alive, number of ventilator-free
days and alive, and number of respiratory failure-free days and
alive up to Day 28 by treatment arm - Efficacy population

| | | | RIPK1 Inhibitor | | | |
|---|---|---|---|---|---|---|
| | Placebo | | 600 mg | | All | |
| | (N = 19) | | (N = 41) | | (N = 60) | |
| Number of Days | | | | | | |
| without need for | | | | | | |
| Oxygen Support | | | | | | |
| and Alive (DAYS) | | | | | | |
| Number | 19 | | 41 | | 60 | |
| Mean (SD) | 18.0 | (10.2) | 20.5 | (7.7) | 19.7 | (8.6) |
| Median | 22.0 | | 23.0 | | 23.0 | |
| Q1; Q3 | 12.0; 25.0 | | 20.0; 25.0 | | 18.5; 25.0 | |
| Min; Max | 0; 27 | | 0; 28 | | 0; 28 | |
| Number of | | | | | | |
| Ventilator-Free | | | | | | |
| Days and Alive | | | | | | |
| (DAYS) | | | | | | |
| Number | 19 | | 41 | | 60 | |
| Mean (SD) | 23.4 | (10.0) | 26.0 | (7.4) | 25.1 | (8.3) |
| Median | 28.0 | | 28.0 | | 28.0 | |
| Q1; Q3 | 28.0; 28.0 | | 28.0; 28.0 | | 28.0; 28.0 | |
| Min; Max | 0; 28 | | 0; 28 | | 0; 28 | |
| Number of | | | | | | |
| Respiratory | | | | | | |
| Failure-Free | | | | | | |
| Days and | | | | | | |
| Alive (DAYS) | | | | | | |
| Number | 19 | | 41 | | 60 | |
| Mean (SD) | 23.3 | (10.0) | 25.9 | (7.4) | 25.1 | (8.3) |

| | | | |
|---|---|---|---|
| Median | 28.0 | 28.0 | 28.0 |
| Q1; Q3 | 27.0; 28.0 | 28.0; 28.0 | 28.0; 28.0 |
| Min; Max | 0; 28 | 0; 28 | 0; 28 |

Day without need for oxygen support and alive is defined as any calendar day with oxygen saturation 92% breathing room air.
Ventilator-free day is defined as any calendar day without use of oxygen therapy such non- invasive ventilation, invasive mechanical ventilation or extracorporeal life support.
Respiratory failure is defined as any use of oxygen therapy as high flow nasal cannula with oxygen flow of ≥30 L/min and FiO2 ≥50% or more severe including any use mechanical ventilation.
For participants who died within the 28 days the number of days with event (i.e., off oxygen support, off ventilator, respiratory failure-free) is set to 0.

3.2.2. Additional Secondary Endpoints

3.2.2.1. Change from Baseline in Markers of Inflammation (White Blood Cell Count, Differential Blood Lymphocytes, Neutrophil to Lymphocyte Ratio) and D-Dimer at Day 7 and End of Treatment (EOT)

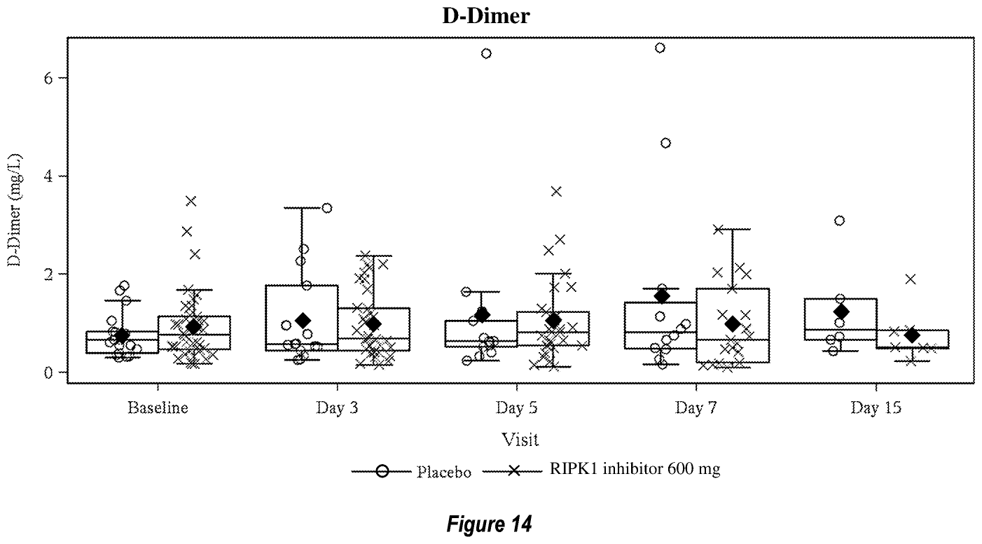

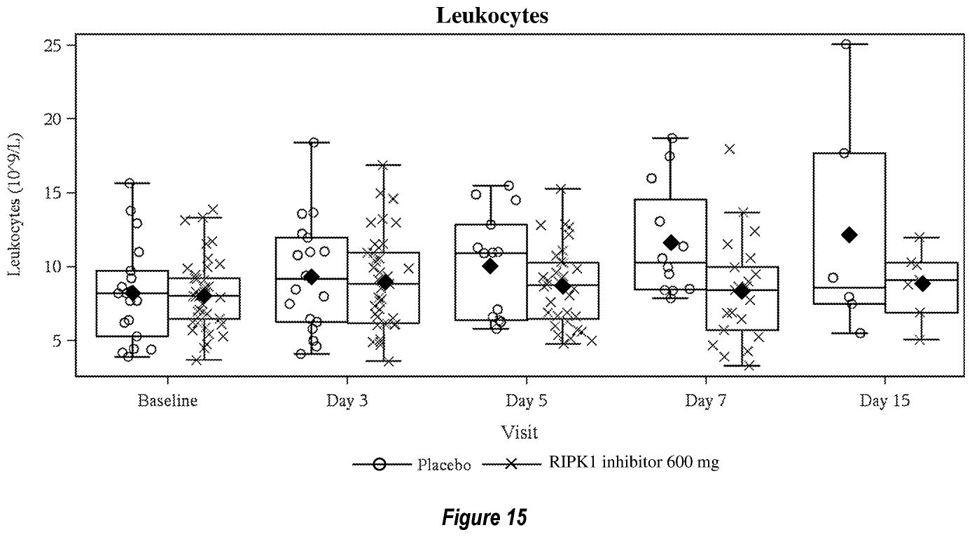

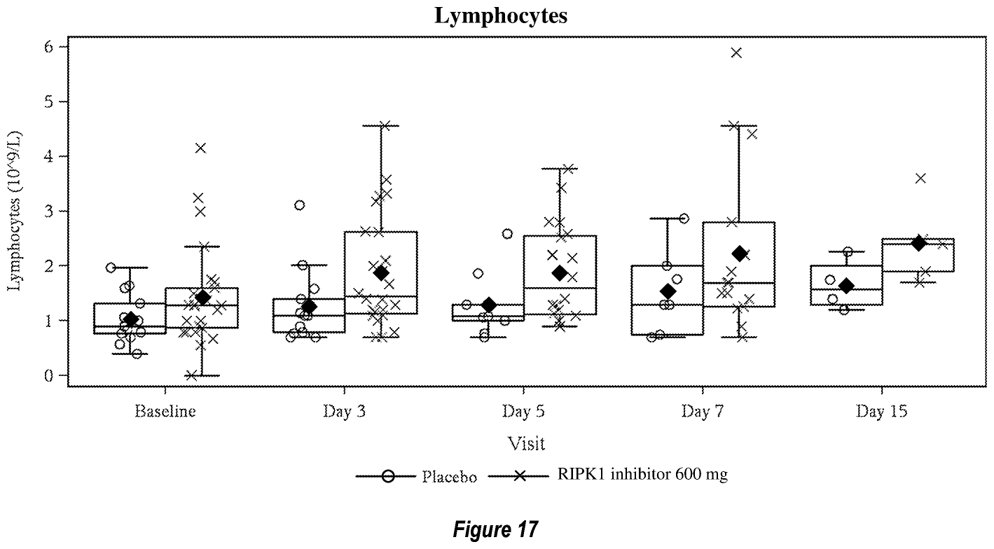

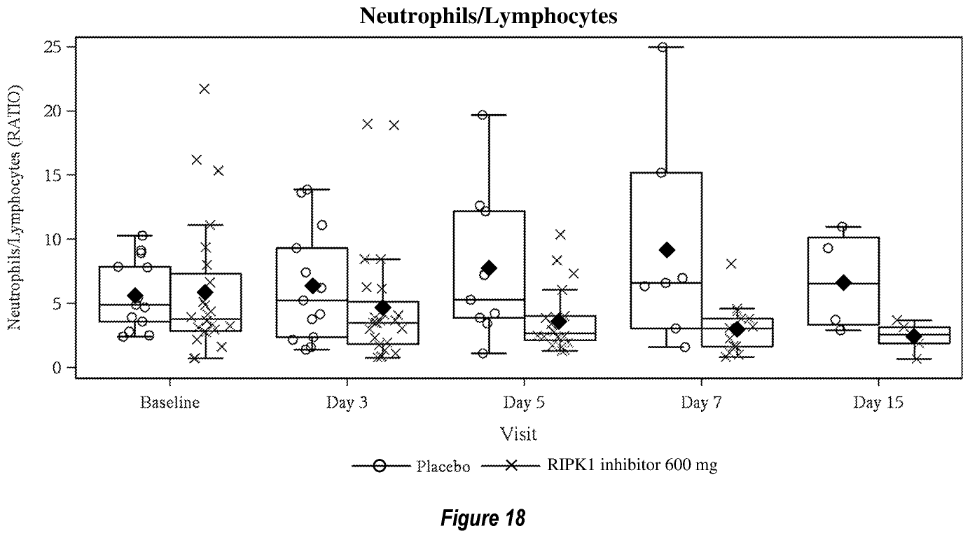

The relative changes from baseline in laboratory markers of severe COVID-19 were analyzed for the two treatment groups and for the treatment comparison of RIPK1 Inhibitor versus placebo, at Day 7 and EOT (Table 22, Table 23, Table 24). See also FIGS. 14, 15, 16, 17, 18, and 19.

Numerically larger decreases in the adjusted geometric means of relative changes from baseline were observed in the RIPK1 Inhibitor versus placebo for: leukocytes at Day 7 only (0.87; 90% CI: 0.73 to 1.03), neutrophils/lymphocytes ratio at Day 7 (0.65; 90% CI: 0.42 to 1.00) and at EOT (0.67; 90% CI: 0.44 to 1.02) (Table 22).

No differences with RIPK1 Inhibitor versus placebo were observed for the other markers. Of note, high neutrophil counts and marked lymphopenia (i.e., elevated neutrophils/lymphocytes ratio) are associated with severe COVID-19 disease and the risk of developing sepsis with rapid progression.

TABLE 22
Laboratory markers of severe COVID-19 - Point estimates of the treatment difference
between RIPK1 Inhibitor and placebo at Day7 and EOT in relative change from
baseline with two-sided 90% confidence interval - Efficacy population

| | | Point | |
|---|---|---|---|
| Parameter | Comparison | estimate | 90% CI |
| D-Dimer | RIPK1 Inhibitor vs placebo at Day 7 | 0.88 | (0.63 to 1.21) |
| | RIPK1 Inhibitor vs placebo at EOT | 1.07 | (0.73 to 1.58) |
| Leukocytes | RIPK1 Inhibitor vs placebo at Day 7 | 0.87 | (0.73 to 1.03) |
| | RIPK1 Inhibitor vs placebo at EOT | 1.03 | (0.86 to 1.23) |
| Lymphocytes | RIPK1 Inhibitor vs placebo at Day 7 | 1.02 | (0.75 to 1.38) |
| | RIPK1 Inhibitor vs placebo at EOT | 1.03 | (0.78 to 1.37) |
| Neutrophils/ | RIPK1 Inhibitor vs placebo at Day 7 | 0.65 | (0.42 to 1.00) |
| Lymphocytes | RIPK1 Inhibitor vs placebo at EOT | 0.67 | (0.44 to 1.02) |
| (RATIO) | | | |
| Ferritin | RIPK1 Inhibitor vs placebo at Day 7 | 0.96 | (0.78 to 1.19) |
| | RIPK1 Inhibitor vs placebo at EOT | 0.98 | (0.77 to 1.24) |
| Lactate | RIPK1 Inhibitor vs placebo at Day 7 | 0.80 | (0.70 to 0.92) |
| Dehydrogenase | RIPK1 Inhibitor vs placebo at EOT | 0.85 | (0.75 to 0.97) |

EOT: End of treatment, or discharge/early discontinuation up to Day 15
The linear mixed effects model on log (relative change in markers) includes baseline log- marker, visit, treatment group and visit-by-treatment group interaction as fixed effects and sites as a random effect. Repeated measures within participants are modeled with an unstructured residual covariance matrix. Point estimate obtained is back-transformed by exponentiation (point estimate displayed).
Point estimate: a value lower than 1 indicates a larger decrease from baseline in treatment group than in placebo group.
Missing values for the relative change from baseline for Days 3, 5, 7, 15 were replaced following the LOCF approach. When several values are available on a day, the last available and evaluable value is considered for the analysis.

TABLE 23
Laboratory markers of severe COVID-19 - Point estimates of
the relative change from baseline (geometric means) with
two-sided 90% confidence interval - Efficacy population

| | | Point | |
|---|---|---|---|
| Parameter | Label | estimate | 90% CI |
| D-Dimer | Placebo at Day 03 | 1.09 | (0.89 to 1.34) |
| | Placebo at Day 05 | 1.11 | (0.88 to 1.41) |
| | Placebo at Day 07 | 1.10 | (0.84 to 1.45) |
| | Placebo at Day 15 | 0.90 | (0.65 to 1.25) |
| | RIPK1 Inhibitor at Day 03 | 1.00 | (0.87 to 1.14) |
| | RIPK1 Inhibitor at Day 05 | 1.04 | (0.89 to 1.22) |
| | RIPK1 Inhibitor at Day 07 | 0.96 | (0.80 to 1.16) |
| | RIPK1 Inhibitor at Day 15 | 0.96 | (0.77 to 1.20) |
| Leukocytes | Placebo at Day 03 | 6.31 | (4.07 to 9.80) |
| | Placebo at Day 05 | 6.46 | (4.17 to 9.99) |
| | Placebo at Day 07 | 7.09 | (4.56 to 11.01) |
| | Placebo at Day 15 | 6.39 | (4.11 to 9.95) |
| | RIPK1 Inhibitor at Day 03 | 6.10 | (3.95 to 9.41) |
| | RIPK1 Inhibitor at Day 05 | 6.14 | (3.98 to 9.46) |
| | RIPK1 Inhibitor at Day 07 | 6.15 | (3.98 to 9.49) |
| | RIPK1 Inhibitor at Day 15 | 6.60 | (4.27 to 10.20) |
| Lymphocytes | Placebo at Day 03 | 1.19 | (0.97 to 1.46) |
| | Placebo at Day 05 | 1.35 | (1.08 to 1.68) |
| | Placebo at Day 07 | 1.49 | (1.14 to 1.94) |
| | Placebo at Day 15 | 1.58 | (1.23 to 2.04) |
| | RIPK1 Inhibitor at Day 03 | 1.35 | (1.12 to 1.62) |
| | RIPK1 Inhibitor at Day 05 | 1.43 | (1.18 to 1.73) |
| | RIPK1 Inhibitor at Day 07 | 1.52 | (1.22 to 1.89) |
| | RIPK1 Inhibitor at Day 15 | 1.63 | (1.32 to 2.02) |
| Neutrophils/ | Placebo at Day 03 | 2.74 | (1.60 to 4.67) |
| Lymphocytes | Placebo at Day 05 | 2.64 | (1.58 to 4.43) |
| (RATIO) | Placebo at Day 07 | 2.69 | (1.57 to 4.59) |
| | Placebo at Day 15 | 2.48 | (1.46 to 4.21) |
| | RIPK1 Inhibitor at Day 03 | 2.01 | (1.28 to 3.15) |
| | RIPK1 Inhibitor at Day 05 | 1.89 | (1.22 to 2.95) |
| | RIPK1 Inhibitor at Day 07 | 1.74 | (1.11 to 2.74) |
| | RIPK1 Inhibitor at Day 15 | 1.66 | (1.05 to 2.60) |
| Ferritin | Placebo at Day 03 | 3.51 | (1.84 to 6.66) |
| | Placebo at Day 05 | 3.31 | (1.73 to 6.36) |
| | Placebo at Day 07 | 2.90 | (1.51 to 5.58) |
| | Placebo at Day 15 | 2.73 | (1.41 to 5.27) |
| | RIPK1 Inhibitor at Day 03 | 3.43 | (1.84 to 6.38) |
| | RIPK1 Inhibitor at Day 05 | 2.91 | (1.55 to 5.46) |
| | RIPK1 Inhibitor at Day 07 | 2.80 | (1.49 to 5.24) |
| | RIPK1 Inhibitor at Day 15 | 2.66 | (1.42 to 5.01) |
| Lactate | Placebo at Day 03 | 2.68 | (1.15 to 6.25) |
| Dehydrogenase | Placebo at Day 05 | 2.60 | (1.11 to 6.09) |
| | Placebo at Day 07 | 2.62 | (1.12 to 6.13) |
| | Placebo at Day 15 | 2.40 | (1.02 to 5.61) |
| | RIPK1 Inhibitor at Day 03 | 2.42 | (1.03 to 5.65) |
| | RIPK1 Inhibitor at Day 05 | 2.23 | (0.95 to 5.21) |
| | RIPK1 Inhibitor at Day 07 | 2.10 | (0.90 to 4.93) |
| | RIPK1 Inhibitor at Day 15 | 2.05 | (0.87 to 4.79) |

The linear mixed effects model on log (relative change in markers) includes baseline log-marker, visit, treatment group and visit-by-treatment group interaction as fixed effects and sites as a random effect. Repeated measures within participants are modeled with an unstructured residual covariance matrix. Point estimate obtained is back-transformed to original scale by exponentiation (point estimate displayed).
Missing values for the relative change from baseline for Days 3, 5, 7, 15 were replaced following the LOCF approach. When several values are available on a day, the last available and evaluable value is considered for the analysis.

TABLE 24
Laboratory markers of severe COVID-19 - Point estimates of the relative
change from baseline (geometric means) with two-sided 90% confidence
interval displayed as percent change - Efficacy population

| | | Point | |
|---|---|---|---|
| Parameter | Label | estimate | 90% CI |
| D-Dimer | Placebo at Day 03 | 9.23 | (−11.27 to 34.48) |
| | Placebo at Day 05 | 10.99 | (−12.34 to 40.53) |
| | Placebo at Day 07 | 10.18 | (−16.26 to 44.96) |
| | Placebo at Day 15 | −10.04 | (−35.24 to 24.97) |
| | RIPK1 Inhibitor at Day 03 | −0.31 | (−13.10 to 14.35) |
| | RIPK1 Inhibitor at Day 05 | 4.10 | (−11.38 to 22.28) |
| | RIPK1 Inhibitor at Day 07 | −3.52 | (−20.09 to 16.50) |
| | RIPK1 Inhibitor at Day 15 | −3.71 | (−23.05 to 20.49) |
| Leukocytes | Placebo at Day 03 | 531.36 | (306.78 to 879.93) |
| | Placebo at Day 05 | 545.54 | (317.02 to 899.27) |
| | Placebo at Day 07 | 608.54 | (355.89 to 1001.18) |
| | Placebo at Day 15 | 539.03 | (310.51 to 894.78) |
| | RIPK1 Inhibitor at Day 03 | 509.93 | (295.21 to 841.31) |
| | RIPK1 Inhibitor at Day 05 | 513.77 | (298.19 to 846.05) |
| | RIPK1 Inhibitor at Day 07 | 514.60 | (297.99 to 849.11) |
| | RIPK1 Inhibitor at Day 15 | 560.21 | (327.20 to 920.32) |
| Lymphocytes | Placebo at Day 03 | 19.32 | (−2.77 to 46.43) |
| | Placebo at Day 05 | 34.85 | (8.01 to 68.36) |
| | Placebo at Day 07 | 48.76 | (14.23 to 93.73) |
| | Placebo at Day 15 | 58.25 | (23.00 to 103.60) |
| | RIPK1 Inhibitor at Day 03 | 34.70 | (12.13 to 61.82) |
| | RIPK1 Inhibitor at Day 05 | 42.51 | (17.52 to 72.81) |
| | RIPK1 Inhibitor at Day 07 | 51.69 | (21.99 to 88.63) |
| | RIPK1 Inhibitor at Day 15 | 63.48 | (32.34 to 101.94) |
| Neutrophils/ | Placebo at Day 03 | 173.89 | (60.46 to 367.49) |
| Lymphocytes | Placebo at Day 05 | 164.28 | (57.54 to 343.33) |
| (RATIO) | Placebo at Day 07 | 168.57 | (57.10 to 359.12) |
| | Placebo at Day 15 | 147.64 | (45.62 to 321.14) |
| | RIPK1 Inhibitor at Day 03 | 100.80 | (27.81 to 215.46) |
| | RIPK1 Inhibitor at Day 05 | 89.49 | (21.85 to 194.67) |
| | RIPK1 Inhibitor at Day 07 | 74.47 | (10.91 to 174.44) |
| | RIPK1 Inhibitor at Day 15 | 65.55 | (5.47 to 159.85) |
| Ferritin | Placebo at Day 03 | 250.53 | (84.45 to 566.17) |
| | Placebo at Day 05 | 231.35 | (72.51 to 536.44) |
| | Placebo at Day 07 | 190.25 | (51.10 to 457.54) |
| | Placebo at Day 15 | 172.70 | (41.04 to 427.29) |
| | RIPK1 Inhibitor at Day 03 | 242.68 | (84.04 to 538.04) |
| | RIPK1 Inhibitor at Day 05 | 191.34 | (55.46 to 445.97) |
| | RIPK1 Inhibitor at Day 07 | 179.61 | (49.19 to 424.02) |
| | RIPK1 Inhibitor at Day 15 | 166.34 | (41.67 to 400.71) |
| Lactate | Placebo at Day 03 | 168.26 | (15.05 to 525.50) |
| Dehydrogenase | Placebo at Day 05 | 160.40 | (11.42 to 508.61) |
| | Placebo at Day 07 | 162.10 | (12.02 to 513.27) |
| | Placebo at Day 15 | 139.68 | (2.47 to 460.65) |
| | RIPK1 Inhibitor at Day 03 | 141.68 | (3.40 to 464.88) |
| | RIPK1 Inhibitor at Day 05 | 122.59 | (−4.91 to 421.02) |
| | RIPK1 Inhibitor at Day 07 | 110.26 | (−10.24 to 392.53) |
| | RIPK1 Inhibitor at Day 15 | 104.58 | (−12.66 to 379.18) |

The linear mixed effects model on log (relative change in markers) includes baseline log-marker, visit, treatment group and visit-by-treatment group interaction as fixed effects and sites as a random effect. Repeated measures within participants are modeled with an unstructured residual covariance matrix. Point estimate obtained is back-transformed to original scale by exponentiation. The percent change is obtained by subtracting 1 from the antilog transformation and multiplying it by 100.
Point estimate (i.e., percent change): a negative value indicates a decrease from baseline. Missing values for the relative change from baseline in CRP for Days 3, 5, 7, 15 were replaced following the LOCF approach. When several values are available on a day, the last available and evaluable value is considered for the analysis.

3.2.2.2. Percentage of Participants Receiving Thrombolytic and Vasopressor Treatment Up to Day 28

The number (percentage) of participants receiving anti-thrombotic treatment up to Day 28 were similar between RIPK1 Inhibitor group (n=20 [48.8%]) and placebo group (n=8 [42.1%]).

A lower number of participants receiving treatment of vasopressor was observed in the RIPK1 Inhibitor treatment group (n=1 [2.4%]) over the placebo group (n=3 [15.8%]).

TABLE 25
Anti-Thrombotics & Vasopressor treatment - Number (%) of participants
receiving treatments up to Day 28 - Efficacy population

| Category for | | |
|---|---|---|
| medication | | RIPK1 Inhibitor |
| Reason for | Placebo | 600 mg |
| treatment | (N = 19) | (N = 41) |
| anti-Thrombotics | 8 (42.1) | 20 (48.8) |
| Prophylaxis | 8 (42.1) | 18 (43.9) |
| Adverse Event | 0 | 3 (7.3) |
| Vasopressor | 3 (15.8) | 1 (2.4) |

n (%) = number and percentage of participants with at least one concomitant medication Categories for medication are sorted by decreasing frequency in SAR441322 600 mg group Reasons for treatment are sorted by decreasing frequency in SAR441322 600 mg group within each category for medication
Note:
A participant can be counted in several categories, but not more than once within a given category.
A patient treated with RIPK1 Inhibitor required Vasopressor treatment at visits excluded from the efficacy analysis due to administration of an anti-IL-6 drug and is therefore not displayed in the table.

3.3. Exploratory Efficacy/Pharmacodynamics Endpoints

3.3.1. Change from Baseline in Ferritin and Lactate-Dehydrogenase (LDH) at Day 7 and EOT

Numerically larger decreases with RIPK1 Inhibitor versus placebo in relative change from baseline were observed for LDH at Day 7 (0.80; 90% CI: 0.70 to 0.92) and at EOT (0.85; 90% CI: 0.75 to 0.97) (Table 22). As a reference, high baseline level and increase in LDH are associated with COVID-19 disease progression and poor outcomes.

No differences with RIPK1 Inhibitor versus placebo were observed for ferritin (Table 22).

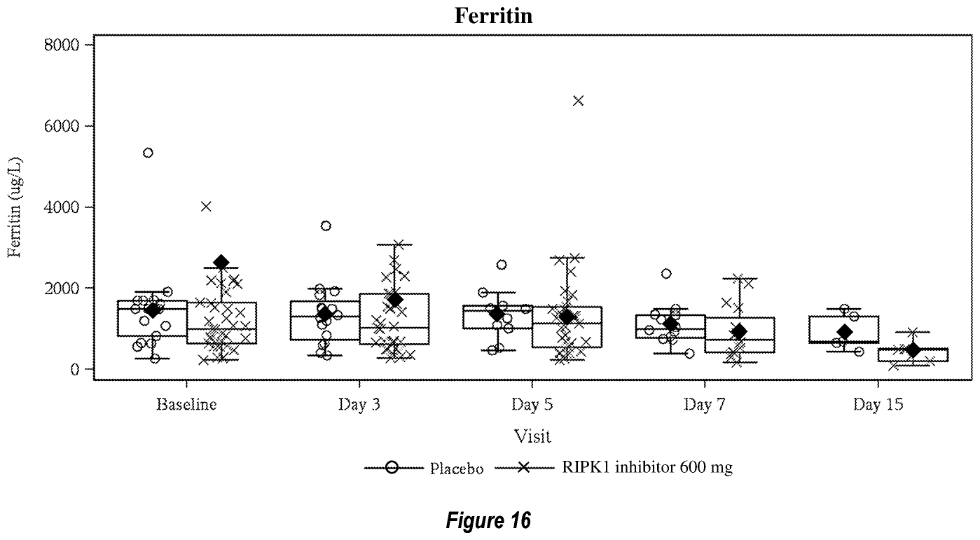

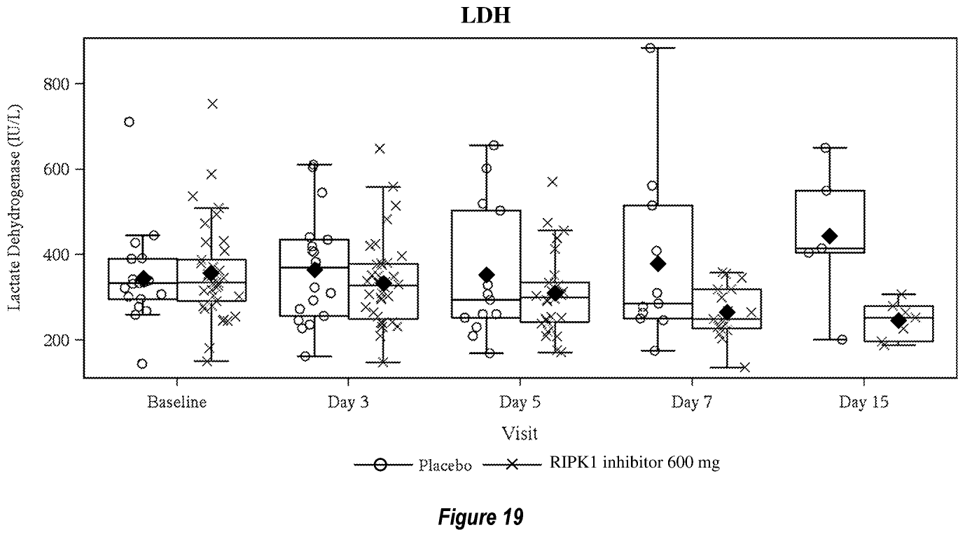

The boxplots of raw values over time for LDH and ferritin are provided in FIG. 19 and FIG. 16, respectively.

3.3.2. Assessment of 7-Point Clinical Scale

3.3.2.1. Proportion of Participants Per Category of the 7-Point Clinical Scale at EOT

All study participants at baseline had a score of 4 (hospitalized, requiring supplemental oxygen). At the end of study treatment period or at the time of early study discontinuation (prior to EOT day/Day 15), in the placebo and the RIPK1 Inhibitor groups, respectively, there were 37% and 15% participants with a score of 5 or lower (5=hospitalized, not requiring supplemental oxygen—requiring ongoing medical care to 1=Death); and 63% and 85% with a score of 7 (not hospitalized) (Table 26). Of note, 3 (16%) participants in the placebo group and one (2%) participant in the active group had a worsening of their condition score down to 2 (hospitalized on invasive mechanical ventilation or ECMO).

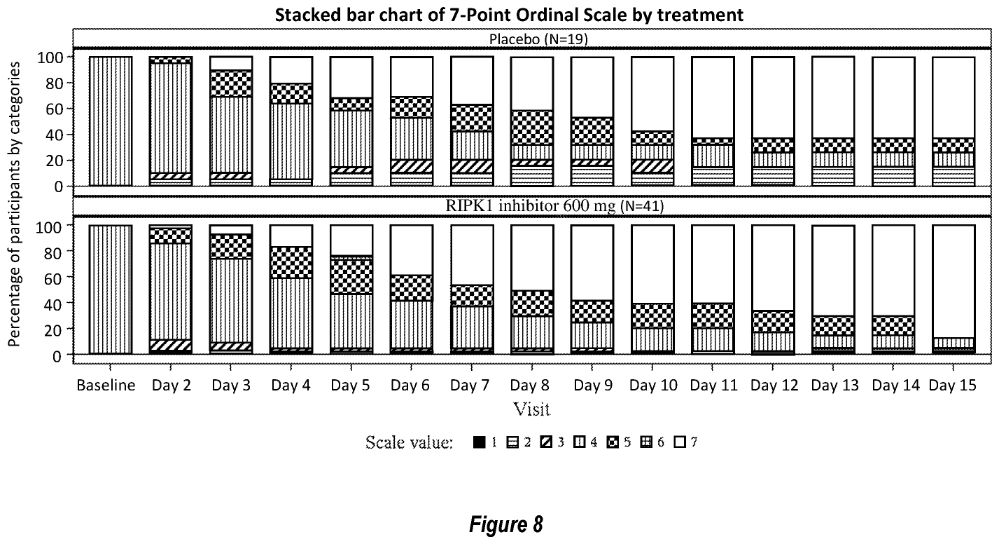

The 7-point scale stacked bar plot of the percentage of participants per category over treatment period including LOCF imputation is visually reflecting a quicker and increased improvement of the participants' condition over the 15-day treatment period (FIG. 8).

TABLE 26
7-point clinical scale - Number (%) of participants per
category at Baseline and EOT - Efficacy population

| | | |
|---|---|---|
| Study day | | RIPK1 Inhibitor |
| 7-point clinical | Placebo | 600 mg |
| scale [n(%)] | (N = 19) | (N = 41) |

| | | |
|---|---|---|
| Baseline | | |
| 1 | 0 | 0 |
| 2 | 0 | 0 |
| 3 | 0 | 0 |

| | | | | |
|---|---|---|---|---|
| 4 | 19 | (100) | 41 | (100) |

| 5 | 0 | 0 | |
|---|---|---|---|
| 6 | 0 | 0 | |
| 7 | 0 | 0 | |
| EOT | | | |
| 1 | 0 | 1 | (2.4) |

| | | | | |
|---|---|---|---|---|
| 2 | 3 | (15.8) | 1 | (2.4) |

| | | |
|---|---|---|
| 3 | 0 | 0 |

| | | | | |
|---|---|---|---|---|
| 4 | 2 | (10.5) | 4 | (9.8) |

| | | | |
|---|---|---|---|
| 5 | 2 | (10.5) | 0 |

| | | |
|---|---|---|
| 6 | 0 | 0 |

| | | | | |
|---|---|---|---|---|
| 7 | 12 | (63.2) | 35 | (85.4) |

EOT: End of treatment, or discharge/early discontinuation up to Day 15
1 = Death,
2 = Hospitalized, on invasive mechanical ventilation or ECMO,
3 = Hospitalized, on non-invasive ventilation or high flow oxygen devices,
4 = Hospitalized, requiring supplemental oxygen,
5 = Hospitalized, not requiring supplemental oxygen - requiring ongoing medical care (COVID-19 related or otherwise),
6 = Hospitalized, not requiring supplemental oxygen - no longer requires ongoing medical care,
7 = Not hospitalized
Note:
When several values for 7-point clinical scale are available on a day, the last available and evaluable value is considered for the analysis.
On the day of hospital discharge due to recovery, the value for 7-point clinical scale is defined as “7 - not hospitalized” by default.

3.3.2.2. Time to Improvement by 2 Points in Category of 7-Point Clinical Scale

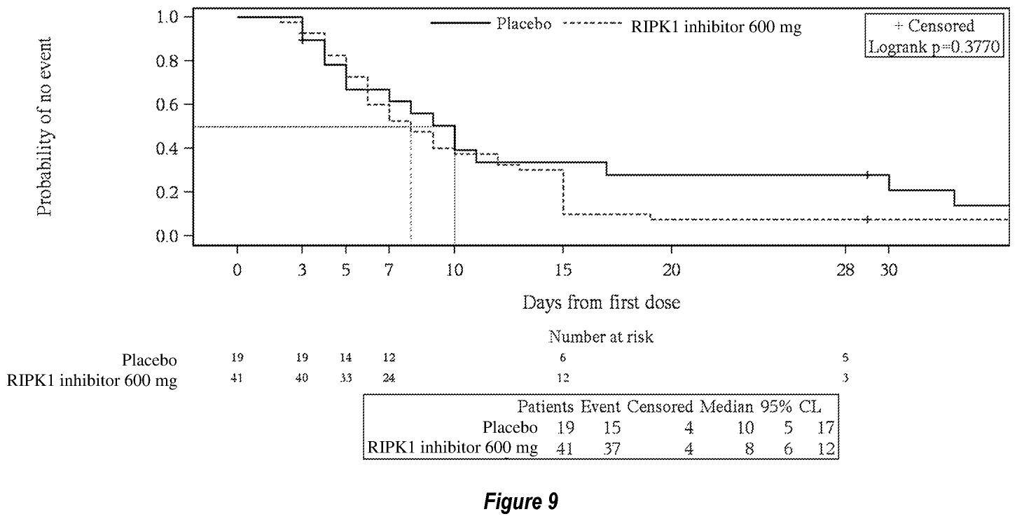

The median time to improvement by at least 2 points in the category of 7-point scale as observed in the KM graph is 10 days for the placebo arm and 8 days in the RIPK1 Inhibitor arm (FIG. 9). The difference in the time to improvement was not statistically significant, supported by the exploratory p-value of the difference between KM curves (0.377).

3.3.3. Change from Baseline in Peripheral Cytokine and Biomarker Levels Up to EOT

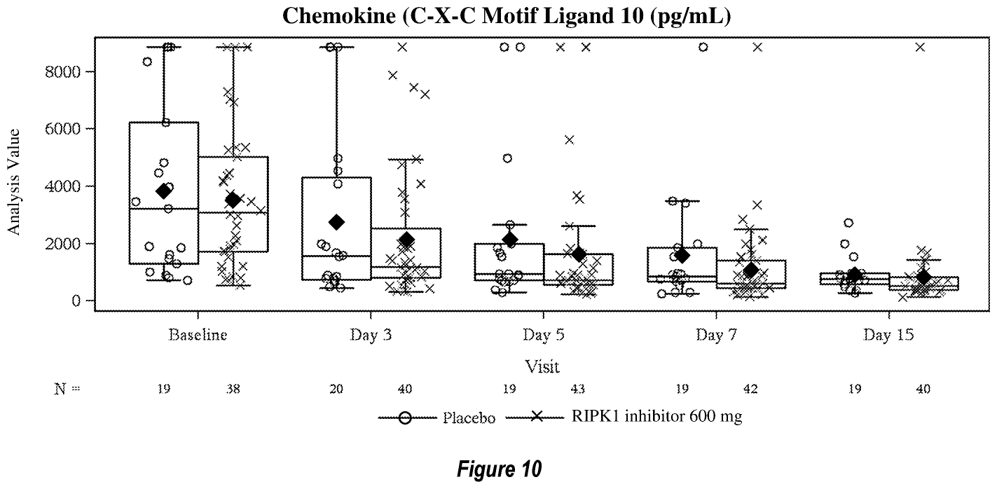

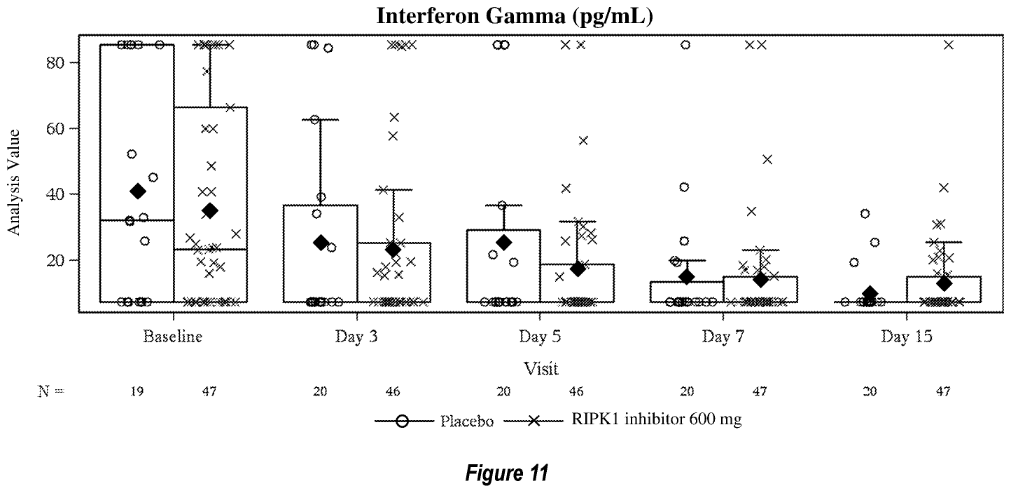

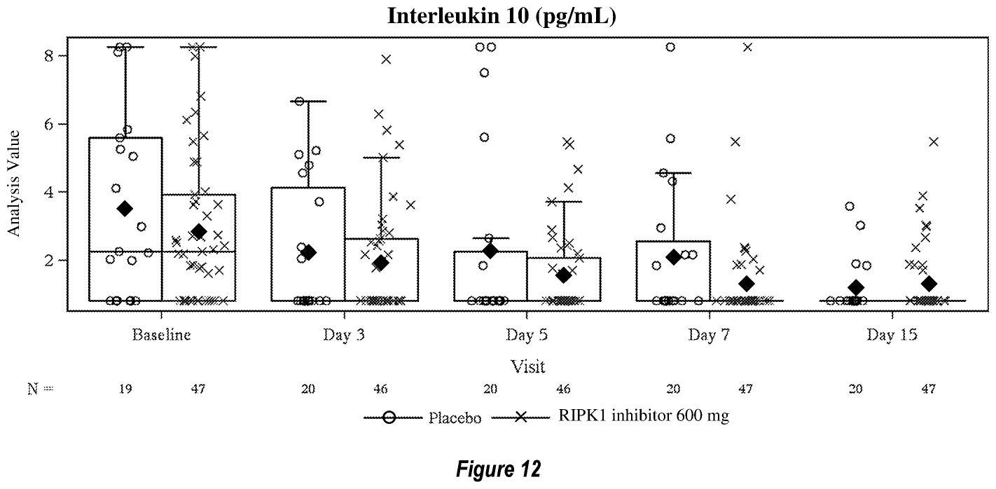

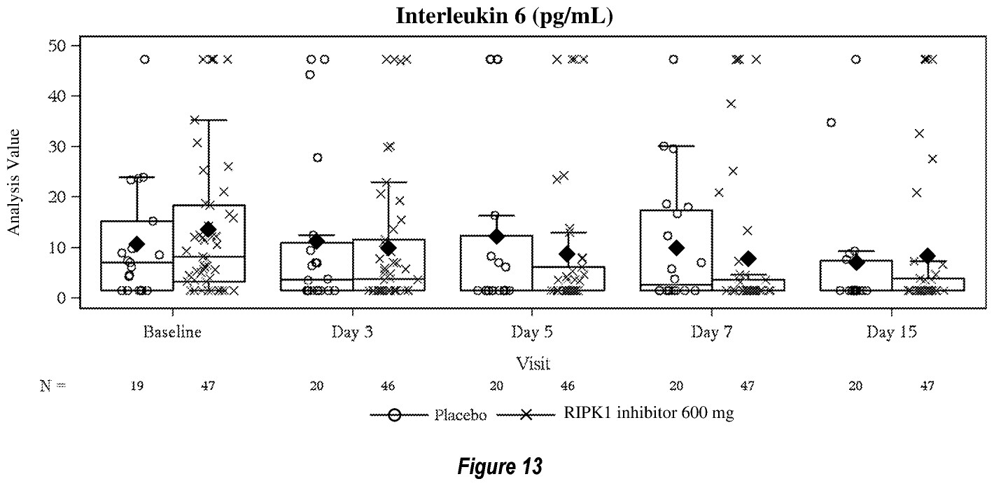

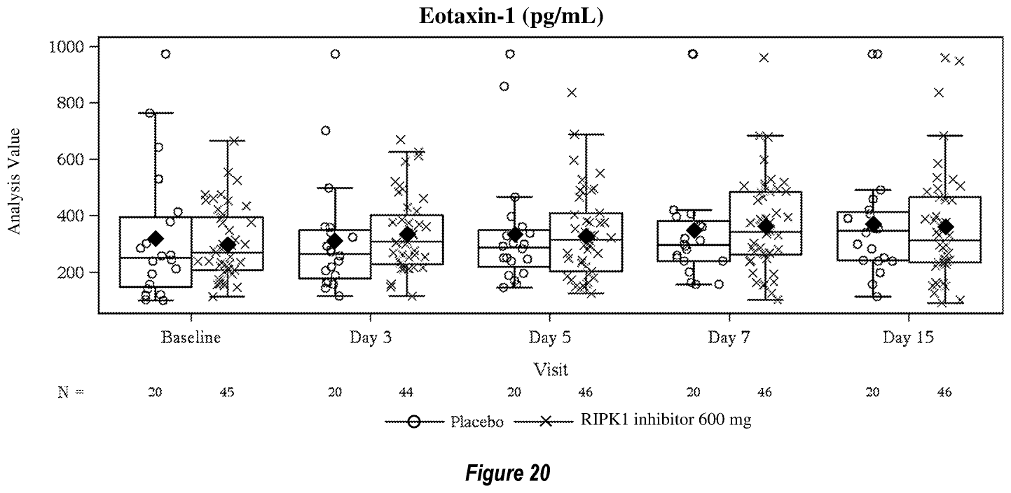

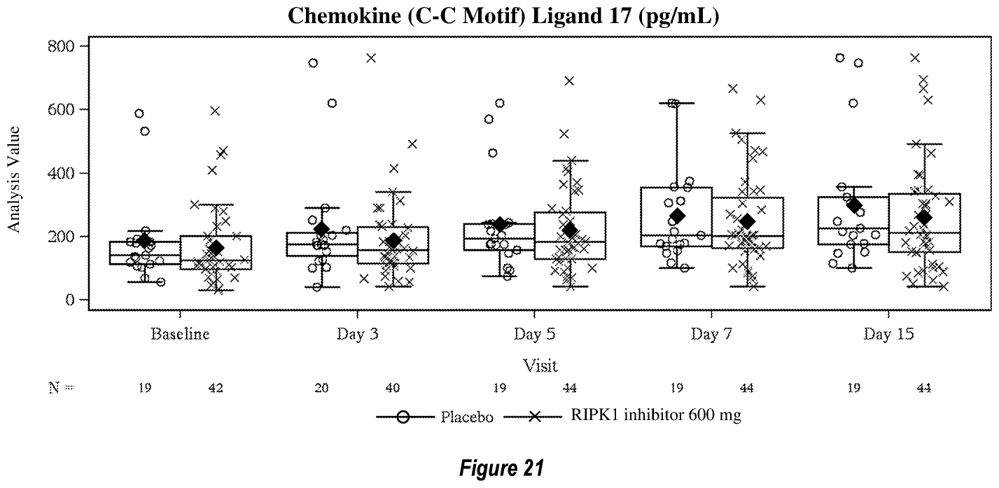

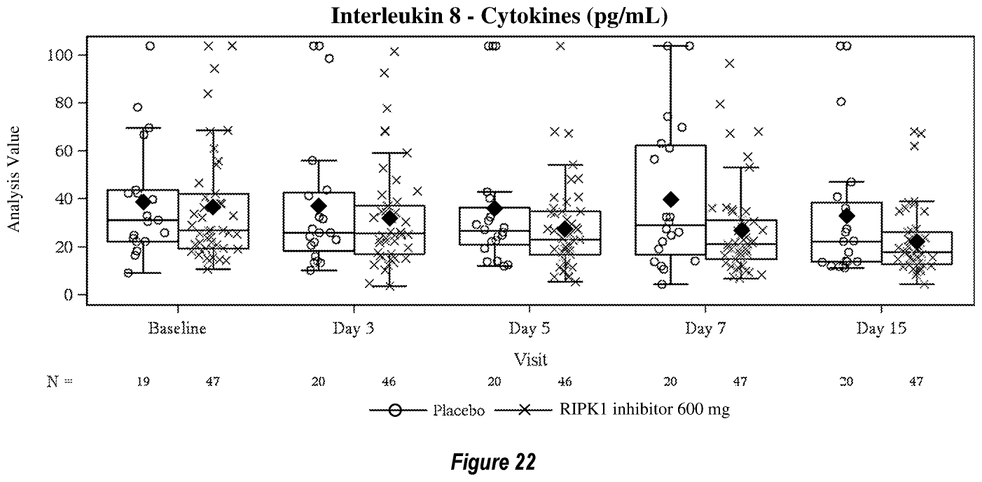

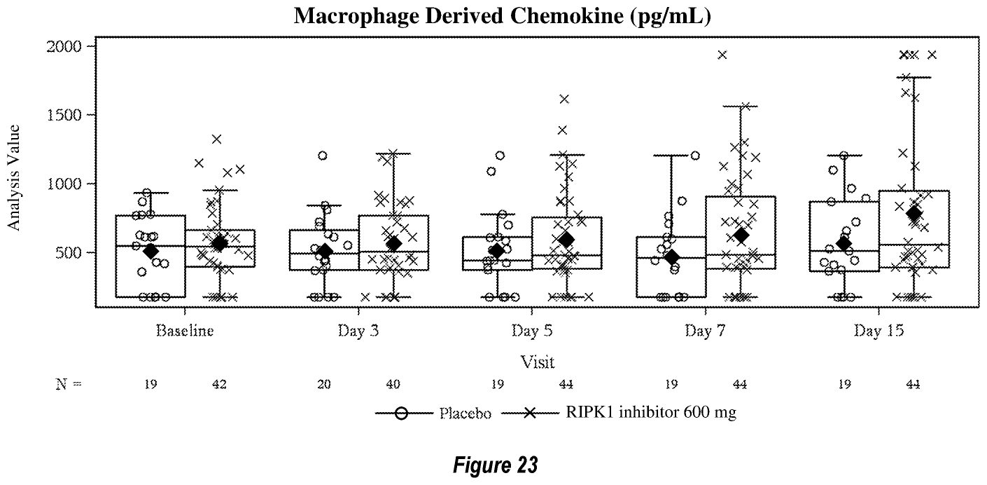

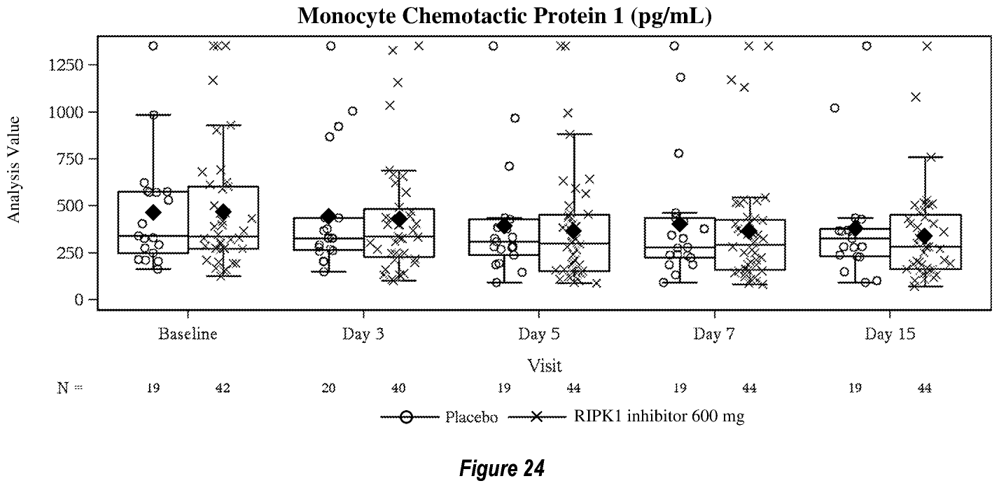

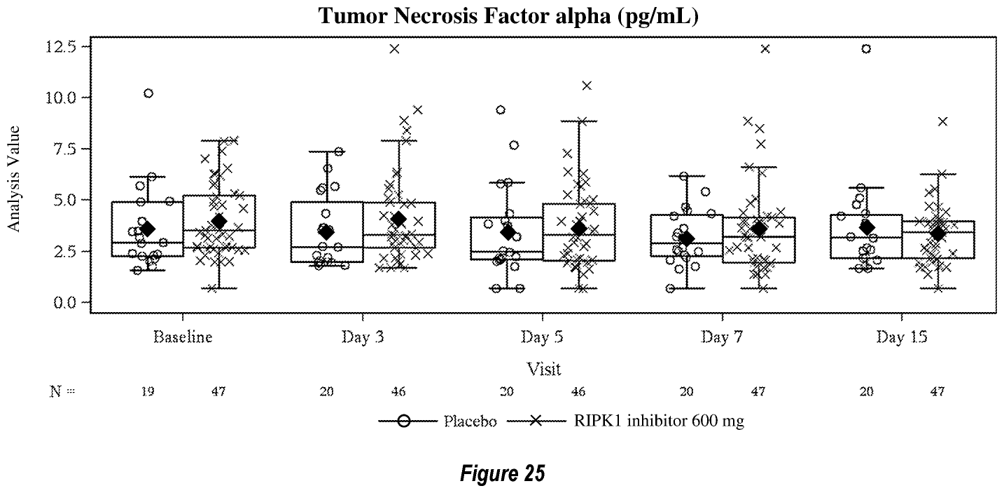

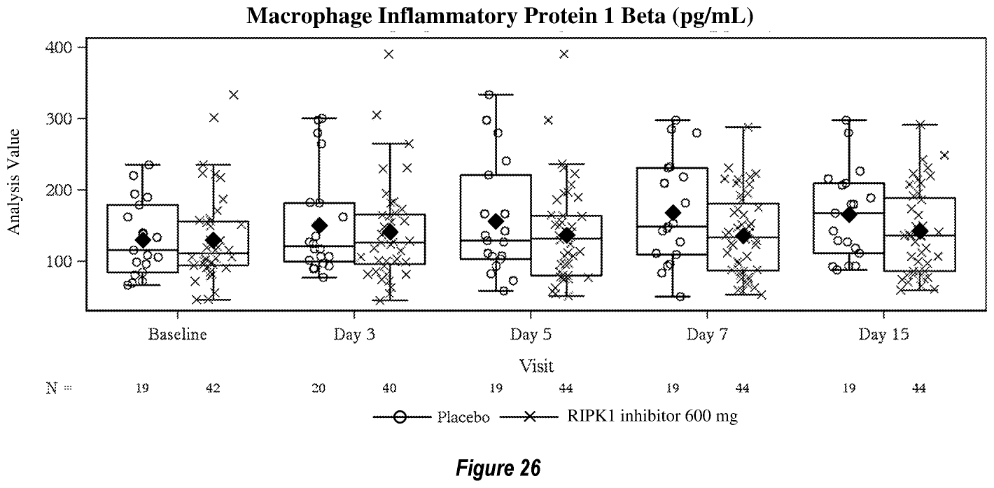

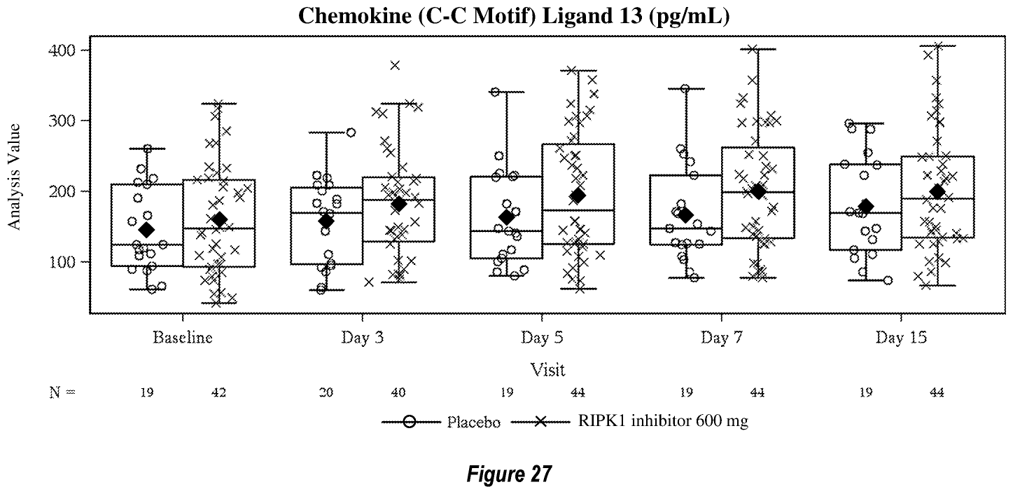

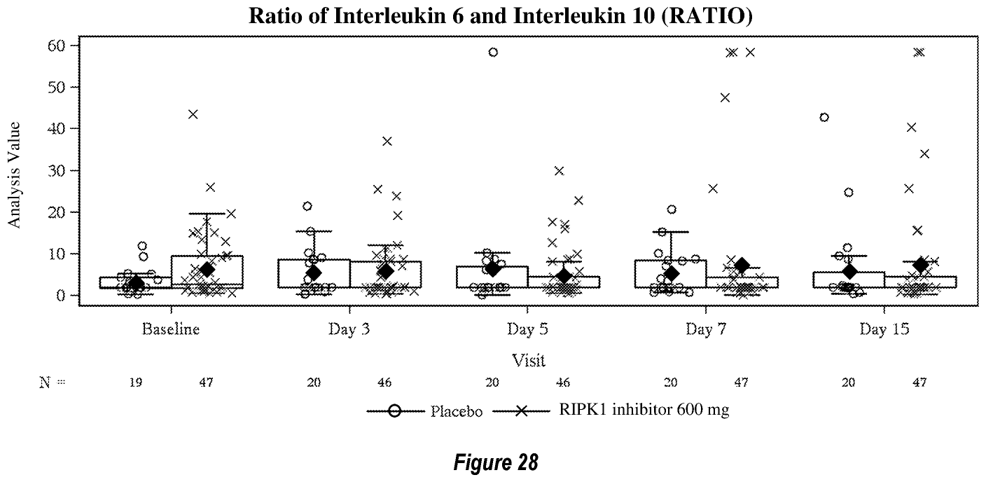

The relative changes from baseline in peripheral cytokine and biomarkers were analyzed for the two treatment groups over time up to EOT (Day 15), and some numerically important reduction in the mean values of chemokine (C-X-C motif) Ligand 10 (FIG. 10), interferon gamma (FIG. 11), IL-10 (FIG. 12), and IL-6 (FIG. 13) were observed in both treatment groups by as early as study Day 3. Boxplots of other biomarkers are provided in FIG. 20, FIG. 21, FIG. 22, FIG. 23, FIG. 24, FIG. 25, FIG. 26, FIG. 27, FIG. 28.

At Day 7, decrease from baseline for these biomarkers were statistically significant with missing data imputed with LOCF approach for placebo and RIPK1 Inhibitor (Table 27):
for interferon gamma, the fold change was 0.43 (p<0.0001) for placebo group and 0.44 (p<0.0001) for RIPK1 Inhibitor group,
for chemokine (C-X-C motif) Ligand 10, the fold change was 0.37 (p<0.0001) for placebo group and 0.26 (p<0.0001) for RIPK1 Inhibitor group,
for IL-10, the fold change was 0.58 (p=0.000159) for placebo group and 0.48 (p=2.311e-12) for RIPK1 Inhibitor group,
for IL-6, the fold change was 0.4 (p<0.0001) for RIPK1 Inhibitor group; Of note, the fold change 0.64 (p=0.0886) in IL-6 for placebo group was not statistically significant.

Furthermore, a numerically greater reduction in chemokine (C-X-C Motif) Ligand 10, IL-10, and IL-6, was observed in the RIPK1 Inhibitor group over placebo, with the ratio of relatives changes (RIPK1 Inhibitor versus placebo) of 0.7, 0.82, and 0.63, respectively (Table 27). However, the differences were not statistically significant.

In addition, although not statistically significant, a greater decrease in monocyte chemotactic protein 1 was observed in favor of RIPK1 Inhibitor group over the placebo arm, the ratio of fold changes between RIPK1 Inhibitor and placebo was 0.85.

TABLE 27
Summary of pharmacodynamic model at Day 7 - Safety population

| | | | RIPK1 | | RIPK1 | RIPK1 |
|---|---|---|---|---|---|---|
| | Placebo | | Inhibitor | RIPK1 | Inhibitor | Inhibitor 600 |
| | Fold- | | 600 mg | Inhibitor 600 | 600 mg vs | mg |
| | Change | Placebo | Fold- | mg | Placebo Fold- | vs Placebo |
| Biomarker | (n) | P-value/FDR | Change (n) | P-value/FDR | Change (n) | P-value/FDR |
| Tumor Necrosis Factor alpha | 0.87 (19) | 0.154/0.2 | 0.85 (47) | 0.0113/0.0146 | 0.98 (66) | 0.86/0.942 |
| (pg/mL) | | | | | | |
| Chemokine (C-C Motif) Ligand | 1.15 (19) | 0.0612/0.133 | 1.31 (41) | 5.315e−07/1.152e−06 | 1.15 (60) | 0.115/0.452 |
| 13 (pg/mL) | | | | | | |
| [symbol] okine (C-C Motif) Ligand 17 | 1.55 (19) | 3.791e−05/0.000164 | 1.56 (41) | 1.334e−08/4.337e−08 | 1 (60) | 0.979/0.979 |
| [symbol] L) | | | | | | |
| Monocyte Chemotactic Protein 1 | 0.82 (19) | 0.131/0.19 | 0.69 (41) | 0.000137/0.000254 | 0.85 (60) | 0.304/0.495 |
| (pg/mL) | | | | | | |
| Macrophage-Derived Chemokine | 0.88 (19) | 0.347/0.408 | 1.05 (41) | 0.572/0.572 | 1.2 (60) | 0.275/0.495 |
| (pg/mL) | | | | | | |
| Interferon Gamma (pg/mL) | 0.43 (19) | 3.942e−07/2.562e−06 | 0.44 (47) | 3.096e−12/1.342e−11 | 1.03 (66) | 0.87/0.942 |
| Ratio of Interleukin 6 and | 1.01 (19) | 0.971/0.971 | 0.87 (47) | 0.415/0.449 | 0.86 (66) | 0.643/0.929 |
| Interleukin 10 (RATIO) | | | | | | |
| Macrophage Inflammatory | 1.26 (19) | 0.0108/0.0281 | 1.07 (41) | 0.233/0.276 | 0.85 (60) | 0.139/0.452 |
| Protein 1 Beta (pg/mL) | | | | | | |
| Interleukin 10 (pg/mL) | 0.58 (19) | 0.000159/0.000515 | 0.48 (47) | 2.311e−12/1.342e−11 | 0.82 (66) | 0.213/0.462 |
| Interleukin 6 (pg/mL) | 0.64 (19) | 0.0886/0.144 | 0.4 (47) | 4.891e−07/1.152e−06 | 0.63 (66) | 0.129/0.452 |
| Interleukin 8 - Cytokines (pg/mL) | 0.88 (19) | 0.377/0.408 | 0.71 (47) | 0.000216/0.000351 | 0.8 (66) | 0.181/0.462 |
| Eotaxin-1 (pg/mL) | 1.17 (20) | 0.0888/0.144 | 1.21 (45) | 0.00264/0.00381 | 1.03 (65) | 0.766/0.942 |
| Chemokine (C-X-C Motif) | 0.37 (19) | 2.206e−08/2.868e−07 | 0.26 (37) | 2.765e−17/3.594e−16 | 0.7 (56) | 0.066/0.452 |
| Ligand 10 (pg/mL) | | | | | | |

Note:
n = Number of patients with baseline and Day 7 assessments. Baseline is defined as the D1 predose assessment value.
Values below LLOQ are replaced by LLOQ/2. Outlier values higher than Q3 + 3 IQR are imputed by Q3 + 3 IQR. Missing data are imputed by Last [symbol]
vation Carried Forward (LOCF) method if at least a baseline and a post-baseline value were available. [symbol]
eduled and discharge before Day 15 (treatment period) visits are re-allocated to study visits according to their study day. [symbol]
fixed effect model with treatment as fixed effect, and baseline as covariate on log-transformed absolute change from baseline.
Fold-Changes are calculated using exponential of log-Least Squared means in each treatment arm, and exponential of log-Least Squared means difference between arms.
FDR = False discovery rate adjusted p-value using the Benjamini-Hochberg procedure. [symbol]
indicates data missing or illegible when filed

3.3.4. Quantitative SARS-COV-2 Viral Load in Blood at Baseline and on Day 3, 5, 7 and EOT

Summary of quantitative measurement of SARS-COV-2 plasma viral load over time (at baseline, Day 3, 5, 7, and EOT) is provided in Table 28. An overall trend of decrease viral load and increased number of negative SARS-COV-2 tests were observed over time. Due to a high variability in the viral load values, no interpretation could be drawn for the effect of treatment on the viral load.

TABLE 28
Viral load in plasma - summary of SARS-COV-2 viral
load in blood raw value - Efficacy population

| | | | RIPK1 Inhibitor | | | |
|---|---|---|---|---|---|---|
| | Placebo | | 600 mg | | All | |
| | (N = 19) | | (N = 41) | | (N = 60) | |
| DAY 01 | | | | | | |
| Number | 16 | | 33 | | 49 | |
| INCONCLUSIVE | 1 | (5.3) | 1 | (2.4) | 2 | (3.3) |
| NO SARS-COV2 | 4 | (21.1) | 11 | (26.8) | 15 | (25.0) |
| DETECTED | | | | | | |
| <1660 CP/ML SARS-COV2 | 4 | (21.1) | 14 | (34.1) | 18 | (30.0) |
| POSITIVE RESULT | 7 | (36.8) | 7 | (17.1) | 14 | (23.3) |
| Positive result (copies/mL) | | | | | | |
| Number | 7 | | 7 | | 14 | |
| Mean (SD) | 14677.0 | (25730.0) | 30217.6 | (42992.8) | 22447.3 | (34981.0) |
| Median | 4751.0 | | 7560.0 | | 6155.5 | |
| Q1; Q3 | 2043.0; 10367.0 | | 1960.0; 78562.0 | | 2043.0; 14455.0 | |
| Min; Max | 2018; 72532 | | 1759; 105034 | | 1759; 105034 | |
| DAY 03 | | | | | | |
| Number | 15 | | 34 | | 49 | |
| INCONCLUSIVE | 2 | (10.5) | 2 | (4.9) | 4 | (6.7) |
| NO SARS-COV2 | 4 | (21.1) | 16 | (39.0) | 20 | (33.3) |
| DETECTED | | | | | | |
| <1660 CP/ML SARS-COV2 | 4 | (21.1) | 12 | (29.3) | 16 | (26.7) |
| POSITIVE RESULT | 5 | (26.3) | 4 | (9.8) | 9 | (15.0) |
| Positive result (copies/mL) | | | | | | |
| Number | 5 | | 4 | | 9 | |
| Mean (SD) | 7560.4 | (7482.7) | 7505.0 | (6425.8) | 7535.8 | (6593.9) |
| Median | 3603.0 | | 6332.5 | | 3603.0 | |
| Q1; Q3 | 2434.0; 10316.0 | | 2350.5; 12659.5 | | 2434.0; 10316.0 | |
| Min; Max | 1938; 19511 | | 1784; 15571 | | 1784; 19511 | |
| DAY 07 | | | | | | |
| Number | 10 | | 16 | | 26 | |
| INCONCLUSIVE | 0 | | 2 | (4.9) | 2 | (3.3) |
| NO SARS-COV2 | 6 | (31.6) | 12 | (29.3) | 18 | (30.0) |
| DETECTED | | | | | | |
| <1660 CP/ML SARS-COV2 | 1 | (5.3) | 1 | (2.4) | 2 | (3.3) |
| POSITIVE RESULT | 3 | (15.8) | 1 | (2.4) | 4 | (6.7) |
| Positive result (copies/mL) | | | | | | |
| Number | 3 | | 1 | | 4 | |
| Mean (SD) | 12937.3 | (18742.0) | 6240.0 | (NC) | 11263.0 | (15664.9) |
| Median | 2549.0 | | 6240.0 | | 4394.5 | |
| Q1; Q3 | 1690.0; 34573.0 | | 6240.0; 6240.0 | | 2119.5; 20406.5 | |
| Min; Max | 1690; 34573 | | 6240; 6240 | | 1690; 34573 | |
| EOT | | | | | | |
| Number | 17 | | 33 | | 50 | |
| INCONCLUSIVE | 0 | | 1 | (2.4) | 1 | (1.7) |
| NO SARS-COV2 | 13 | (68.4) | 28 | (68.3) | 41 | (68.3) |
| DETECTED | | | | | | |
| <1660 CP/ML SARS-COV2 | 3 | (15.8) | 4 | (9.8) | 7 | (11.7) |
| POSITIVE RESULT | 1 | (5.3) | 0 | | 1 | (1.7) |
| Positive result (copies/mL) | | | | | | |
| Number | 1 | | 0 | | 1 | |
| Mean (SD) | 3609.0 | (NC) | NC | (NC) | 3609.0 | (NC) |

| | | | |
|---|---|---|---|
| Median | 3609.0 | NC | 3609.0 |
| Q1; Q3 | 3609.0; 3609.0 | NC; NC | 3609.0; 3609.0 |
| Min; Max | 3609; 3609 | NC; NC | 3609; 3609 |

Note:
Baseline is defined as the D1 predose assessment value; CP/ML: copies/mL
Some samples were not analysed by the laboratory due to “insufficient quantity” or “questionable integrity”.

3.4. Efficacy/Pharmacodynamics Conclusions

The primary endpoint (relative change in CRP versus baseline at Day 7) was not met when RIPK1 Inhibitor was compared to placebo added to standard hospital care. Of note, corticosteroids, which are known to decrease CRP levels, were administered as standard of care in approximately 65% of the participants in each treatment group. Although not statistically significant, consistent numerical trends were observed in favor of RIPK1 Inhibitor in the assessment of key secondary and exploratory clinical endpoints.

There is no statistically significant difference in the primary endpoint of relative change in CRP at Day 7 from baseline between the treatment and the placebo groups (p-value: 0.302). However, the relative CRP decrease from baseline is numerically greater in the treatment group as indicated by the ratio of the geometric means of relative change from baseline with RIPK1 Inhibitor versus placebo on Day 7 that equals 0.85 (90% CI: 0.49 to 1.45). A trend toward an earlier decrease in CRP is observed in the KM graph, with the p-value on the difference between KM curves nearing statistical significance with 0.0557. Of note, corticosteroids, which are known to decrease CRP levels, were administered as standard of care in approximately 65% of the participants in each treatment group.

A numerically greater increase (i.e., improvement) was observed in the RIPK1 Inhibitor group versus placebo in the change from baseline in SpO₂/FiO₂ ratio at Day 7. As for CRP, a trend toward an earlier increase in SpO₂/FiO₂ was observed in the KM graph. However, there was no statistically significant difference between RIPK1 Inhibitor group and placebo group.

There was a general trend favoring the RIPK1 Inhibitor treatment group over the placebo group in the observed mean number of days without need of oxygen support, mean VFD, and mean RFFD. Although not statistically significant, numerical trends were consistently observed in favor of RIPK1 Inhibitor in the assessment of key endpoints.

### 4. SAFETY EVALUATION

4.1. Extent of Exposure

Each of the 67 participants in the safety population received their assigned treatment of placebo or RIPK1 Inhibitor 600 mg (Table 29).

The number and percentage of participants grouped by the duration of the study treatment exposure and by treatment group is presented in Table 29. Six (30.0%) participants in the placebo group and 10 (21.3%) participants in the RIPK1 Inhibitor group received study treatment for 14 days.

TABLE 29
Exposure to investigational medicinal product - safety population

| Duration^{a} of | | |
|---|---|---|
| study treatment | | RIPK1 Inhibitor |
| by category | Placebo | 600 mg |
| [n (%)] | (N = 20) | (N = 47) |

| 2 | days | 1 | (5.0) | 4 | (8.5) |
|---|---|---|---|---|---|
| 3 | days | 2 | (10.0) | 2 | (4.3) |
| 4 | days | 2 | (10.0) | 4 | (8.5) |
| 5 | days | 1 | (5.0) | 4 | (8.5) |
| 6 | days | 0 | | 6 | (12.8) |
| 7 | days | 1 | (5.0) | 5 | (10.6) |
| 8 | days | 1 | (5.0) | 1 | (2.1) |
| 9 | days | 1 | (5.0) | 3 | (6.4) |
| 10 | days | 3 | (15.0) | 2 | (4.3) |
| 11 | days | 1 | (5.0) | 1 | (2.1) |
| 12 | days | 1 | (5.0) | 2 | (4.3) |
| 13 | days | 0 | | 3 | (6.4) |
| 14 | days | 6 | (30.0) | 10 | (21.3) |

^{a}Duration = (date of last IMP administration - date of first IMP administration +1);
IMP: Investigational Medicinal Product
n (%) = Number and % of participants having the corresponding duration of exposure
Note:
The denominator is N, the number of participants actually treated within each group.

4.2. Adverse Events

4.2.1. Brief Summary of Adverse Events

An overview of TEAEs is presented in Table 30.

There were 34 participants who reported at least 1 TEAE in the study (10 out of 20 participants in the placebo group and 24 out of 47 participants in the RIPK1 Inhibitor group) (Table 30). The percentage of participants with TEAEs was balanced between the placebo (50.0%) and active treatment (51.1%) arms.

There were 3 participants who reported TEAE leading to death (2 participants in the placebo group and 1 participant in the RIPK1 Inhibitor group), and 1 participant in the RIPK1 Inhibitor group with post-treatment AE leading to death (Table 45), see Section 4.3.1. There were 9 participants who reported at least 1 serious TEAE in the study (3 out of 20 participants in the Placebo group and 6 out of 47 participants in the RIPK1 Inhibitor group), see Section 4.3.2. There were 5 participants who reported at least 1 TEAE leading to permanent study treatment discontinuation in the study (1 out of 20 participants in the placebo group and 4 out of 47 participants in the RIPK1 Inhibitor group), see Section 4.3.3. There were 9 participants who reported at least 1 AESI in the study (3 out of 20 participants in the Placebo group and 6 out of 47 participants in the RIPK1 Inhibitor group) see Section 4.3.4. There were 14 participants who reported at least 1 severe TEAE in the study (6 out of 20 participants in the placebo group and 8 out of 47 participants in the RIPK1 Inhibitor group).

TABLE 30
Overview of adverse event profile: Treatment-
emergent adverse events - Safety population

| | | RIPK1 Inhibitor | |
|---|---|---|---|
| | Placebo | 600 mg | |
| n (%) | (N = 20) | (N = 47) | |
| Participants with any TEAE | 10 (50.0) | 24 | (51.1) |
| Participants with severe TEAE | 6 (30.0) | 8 | (17.0) |
| Participants with any | 3 (15.0) | 6 | (12.8) |
| treatment emergent SAE | | | |
| Participants with any | 2 (10.0) | 1 | (2.1) |
| TEAE leading to death | | | |
| Participants with any | 1 (5.0) | 4 | (8.5) |
| TEAE leading to definitive treatment | | | |
| discontinuation | | | |
| Participants with any | 3 (15.0) | 6 | (12.8) |
| TEAE of special interest (AESI) | | | |
| Participants with any | 3 (15.0) | 1 | (2.1) |
| TEAE related to the compound | | | |

TEAE: Treatment emergent adverse event, SAE: Serious adverse event. N (%) = number and percentage of participants with at least one TEAE.
Note:
Definitive treatment discontinuation is the discontinuation of all study drugs. When all study drugs are not discontinued at the same time, the reason for definitive discontinuation is the reason for discontinuation of the last study drug stopped. Premature discontinuation is the discontinuation of at least one of the study drugs and at least one is continued. An adverse event is considered as treatment emergent if it occurred at the time from first dose of study intervention up to and including the day of last dose of study intervention plus 5 days.

4.2.2. Analysis of Adverse Events

The number (%) of participants with at least 1 TEAE presented by primary SOC and PT is provided in Table 31.

The most frequently reported TEAEs by primary SOC were Gastrointestinal disorders (4 out of 20 [20.0%] participants in the placebo group and 6 out of 47 [12.8%] in the RIPK1 Inhibitor group) and General disorders and administration site conditions (4 out of 20 [20.0%] participants in the placebo group and 6 out of 47 [12.8%] in the RIPK1 Inhibitor group) (Table 31).

The most frequently reported TEAE by PT was condition aggravated (4 out of 20 [20.0%] participants in the placebo group and 4 out of 47 [8.5%] participant in the RIPK1 Inhibitor group), and ALT increased (2 out of 20 [10.0%] participants in the placebo group and 6 out of 47 [12.8%] participant in the RIPK1 Inhibitor group).

A small number of participants reported 8 TEAEs considered as IMP-related by the Investigator: 6 TEAEs in 3 out of 20 [15.0%] participants from the placebo group, and 2 TEAEs in 1 out of 47 [2.1%] participants from RIPK1 Inhibitor group (Table 30). For the most frequently reported TEAEs at PT level, only one TEAE of ALT increased in the placebo group was considered as related to the IMP by the Investigator.

The majority of the TEAEs reported during the study were of grade 2 intensity in the RIPK1 Inhibitor group, and of grade 3 intensity for the placebo group.

TABLE 31
Number (%) of participants with TEAE(s)
by Primary SOC and PT - Safety population

Primary System

| Organ Class | | | RIPK1 Inhibitor | |
|---|---|---|---|---|
| Preferred | Placebo | | 600 mg | |
| Term n(%) | (N = 20) | | (N = 47) | |
| Any class | 10 | (50.0) | 24 | (51.1) |
| INFECTIONS AND INFESTATIONS | 5 | (25.0) | 4 | (8.5) |
| Bacterial infection | 1 | (5.0) | 1 | (2.1) |
| Pneumonia bacterial | 0 | | 1 | (2.1) |
| Pseudomembranous colitis | 0 | | 1 | (2.1) |
| Pseudomonas infection | 0 | | 1 | (2.1) |
| Furuncle | 1 | (5.0) | 0 | |
| Pneumonia | 1 | (5.0) | 0 | |
| Tracheitis | 1 | (5.0) | 0 | |
| Tracheobronchitis | 1 | (5.0) | 0 | |
| BLOOD AND LYMPHATIC SYSTEM | 2 | (10.0) | 1 | (2.1) |
| DISORDERS | | | | |
| Anaemia | 2 | (10.0) | 1 | (2.1) |
| IMMUNE SYSTEM DISORDERS | 0 | | 1 | (2.1) |
| Drug hypersensitivity | 0 | | 1 | (2.1) |
| METABOLISM AND NUTRITION | 1 | (5.0) | 3 | (6.4) |
| DISORDERS | | | | |
| Dehydration | 0 | | 1 | (2.1) |
| Hyperglycaemia | 0 | | 1 | (2.1) |
| Hypoglycaemia | 0 | | 1 | (2.1) |
| Hypophosphataemia | 0 | | 1 | (2.1) |
| Metabolic acidosis | 1 | (5.0) | 0 | |
| PSYCHIATRIC DISORDERS | 0 | | 1 | (2.1) |
| Anxiety disorder | 0 | | 1 | (2.1) |
| NERVOUS SYSTEM DISORDERS | 2 | (10.0) | 0 | |
| Cerebral ischaemia | 1 | (5.0) | 0 | |
| Encephalopathy | 1 | (5.0) | 0 | |
| Psychomotor hyperactivity | 1 | (5.0) | 0 | |
| CARDIAC DISORDERS | 2 | (10.0) | 0 | |
| Cardiac arrest | 1 | (5.0) | 0 | |
| Tachycardia paroxysmal | 1 | (5.0) | 0 | |
| VASCULAR DISORDERS | 0 | | 3 | (6.4) |
| Hypertension | 0 | | 1 | (2.1) |
| Peripheral artery thrombosis | 0 | | 1 | (2.1) |
| Venous thrombosis limb | 0 | | 1 | (2.1) |
| RESPIRATORY, THORACIC AND | 3 | (15.0) | 4 | (8.5) |
| MEDIASTINAL DISORDERS | | | | |
| Dyspnoea | 0 | | 1 | (2.1) |
| Emphysema | 0 | | 1 | (2.1) |
| Oropharyngeal pain | 0 | | 1 | (2.1) |
| Pulmonary embolism | 0 | | 1 | (2.1) |
| Respiratory disorder | 0 | | 1 | (2.1) |
| Noninfective bronchitis | 1 | (5.0) | 0 | |
| Pleural effusion | 1 | (5.0) | 0 | |
| Pneumomediastinum | 2 | (10.0) | 0 | |
| Pneumothorax | 1 | (5.0) | 0 | |
| Respiratory failure | 1 | (5.0) | 0 | |
| GASTROINTESTINAL DISORDERS | 4 | (20.0) | 6 | (12.8) |
| Diarrhoea | 1 | (5.0) | 4 | (8.5) |
| Constipation | 0 | | 1 | (2.1) |
| Flatulence | 0 | | 1 | (2.1) |
| Nausea | 1 | (5.0) | 1 | (2.1) |
| Dyspepsia | 1 | (5.0) | 0 | |
| Gastritis | 1 | (5.0) | 0 | |
| Gastrooesophageal | 1 | (5.0) | 0 | |
| sphincter | | | | |
| insufficiency | | | | |
| Oesophageal ulcer | 1 | (5.0) | 0 | |
| Oesophagitis | 1 | (5.0) | 0 | |
| Pneumoperitoneum | 1 | (5.0) | 0 | |
| Vomiting | 1 | (5.0) | 0 | |
| HEPATOBILIARY DISORDERS | 1 | (5.0) | 0 | |
| Cholelithiasis | 1 | (5.0) | 0 | |
| SKIN AND SUBCUTANEOUS | 2 | (10.0) | 0 | |
| TISSUE DISORDERS | | | | |
| Subcutaneous emphysema | 2 | (10.0) | 0 | |
| MUSCULOSKELETAL AND | 0 | | 1 | (2.1) |
| CONNECTIVE TISSUE | | | | |
| DISORDERS | | | | |
| Back pain | 0 | | 1 | (2.1) |
| RENAL AND URINARY | 2 | (10.0) | 0 | |
| DISORDERS | | | | |
| Renal cyst | 1 | (5.0) | 0 | |
| Renal impairment | 1 | (5.0) | 0 | |
| REPRODUCTIVE SYSTEM AND | 1 | (5.0) | 0 | |
| BREAST DISORDERS | | | | |
| Ovarian cyst | 1 | (5.0) | 0 | |
| GENERAL DISORDERS AND | 4 | (20.0) | 6 | (12.8) |
| ADMINISTRATION SITE | | | | |
| CONDITIONS | | | | |
| Condition aggravated | 4 | (20.0) | 4 | (8.5) |
| Chest discomfort | 0 | | 1 | (2.1) |
| Fatigue | 0 | | 1 | (2.1) |
| Non-cardiac chest pain | 0 | | 1 | (2.1) |
| Pyrexia | 0 | | 1 | (2.1) |
| Vessel puncture site phlebitis | 0 | | 1 | (2.1) |
| INVESTIGATIONS | 4 | (20.0) | 6 | (12.8) |
| Alanine aminotransferase | 2 | (10.0) | 6 | (12.8) |
| increased | | | | |
| Aspartate aminotransferase | 0 | | 1 | (2.1) |
| increased | | | | |
| Blood pressure increased | 1 | (5.0) | 0 | |
| Transaminases increased | 1 | (5.0) | 0 | |
| INJURY, POISONING AND | 1 | (5.0) | 1 | (2.1) |
| PROCEDURAL COMPLICATIONS | | | | |

| | | | |
|---|---|---|---|
| Arterial injury | 0 | 1 | (2.1) |

| | | | |
|---|---|---|---|
| Procedural pneumothorax | 1 | (5.0) | 0 |

TEAE: Treatment emergent adverse event,
SOC: System organ class,
PT: Preferred term MedDRA 23.1
n (%) = number and percentage of participants with at least one TEAE
Note:
Table sorted by SOC internationally agreed order and by decreasing frequency of PT in RIPK1 Inhibitor group
An adverse event is considered as treatment emergent if it occurred at the time from first dose of study intervention up to and including the day of last dose of study intervention plus 5 days.
Preferred term: Condition Aggravated in General disorders and administration site conditions corresponds to worsening of COVID-19.

4.2.2.1. Incidence of Deaths up to 28 Days

Overall, there were 4 (5.9%) deaths due to COVID-19 complication or worsening of COVID-19 during the conduct of the study up to Day 28. Two death cases were reported in the placebo group (10.0%) on Day 18 and Day 20, and 2 participants in the RIPK1 Inhibitor group (4.3%) on Day 11 and Day 15, respectively (Table 32).

TABLE 32
Death - Number and cumulative incidence rate of deaths - Safety population

| | | Participants | Number | Cumulative | Confidence Limits | |
|---|---|---|---|---|---|---|
| Treatment | Study day | at risk | of deaths | incidence rate | Lower | Upper |
| Placebo | 0 | 20 | 0 | 0 | . | . |
| | 18 | 20 | 1 | 0.05 | 0 | 0.21 |
| | 20 | 19 | 1 | 0.1 | 0.02 | 0.28 |
| | 27 | 18 | 0 | 0.1 | 0.02 | 0.28 |
| | 28 | 16 | 0 | 0.1 | 0.02 | 0.28 |
| | 29 | 9 | 0 | 0.1 | 0.02 | 0.28 |
| | 30 | 3 | 0 | 0.1 | 0.02 | 0.28 |
| | 33 | 2 | 0 | 0.1 | 0.02 | 0.28 |
| | 52 | 1 | 0 | 0.1 | 0.02 | 0.28 |
| RIPK1 Inhibitor | 0 | 47 | 0 | 0 | . | . |
| 600 mg | | | | | | |
| | 13 | 47 | 1 | 0.02 | 0 | 0.1 |
| | 15 | 46 | 1 | 0.04 | 0.01 | 0.13 |
| | 27 | 45 | 0 | 0.04 | 0.01 | 0.13 |
| | 28 | 41 | 0 | 0.04 | 0.01 | 0.13 |
| | 29 | 27 | 0 | 0.04 | 0.01 | 0.13 |
| | 30 | 11 | 0 | 0.04 | 0.01 | 0.13 |
| | 31 | 6 | 0 | 0.04 | 0.01 | 0.13 |
| | 33 | 3 | 0 | 0.04 | 0.01 | 0.13 |
| | 42 | 2 | 0 | 0.04 | 0.01 | 0.13 |
| | 60 | 1 | 0 | 0.04 | 0.01 | 0.13 |

4.3. Deaths, Serious Adverse Events, and Other Significant Adverse Events

4.3.1. Deaths

During the study, a total of 4 participants died. All these participants had TEAEs with fatal outcome (start date of the AE was on-treatment with the resulting death occurring either on-treatment or after the end of treatment) (Table 31, Table 45):

In the RIPK1 Inhibitor group:
One participant died due to an SAE of condition aggravated (worsened COVID-19 pneumonia) on study Day 11.
One participant died due to a post-treatment AE of cardiac arrest on study Day 15.

In the placebo group:
One participant died due to a post-treatment AE of condition aggravated (worsened COVID-19 pneumonia) on study Day 20. The onset of the event started during treatment emergent period (Day 5).
One participant died due to an SAE of cardiac arrest on study Day 18.

All TEAEs leading to death were considered as not IMP-related by Investigator.

4.3.2. Serious Adverse Events

Overall, 15 serious TEAEs were reported during the study. All SAEs were assessed as correlated to COVID-19 associated signs, symptoms and/or complications.

In the placebo group, 7 serious TEAEs were reported in 3 participants:
2 in one participant (bacterial infection and respiratory failure),
2 in one participant (2 events of condition aggravated),
3 in one participant (2 events of cardiac arrest and condition aggravated).

In the RIPK1 Inhibitor group, 8 serious TEAEs were reported in 6 participants:
1 in one participant (bacterial infection),
2 in one participant (pneumonia bacterial and pulmonary embolism),
1 in one participant (peripheral artery thrombosis),
1 in one participant (Pseudomonas infection),
1 in one participant (condition aggravated),
2 in one participant (2 events of condition aggravated).

The percentage of participants with any SAE was balanced between the placebo (15.0%) and active treatment (12.8%) arms (Table 33). All SAEs reported during the treatment period were considered as not related to IMP by the Investigators.

TABLE 33
Number (%) of participants with TEAE(s) (SAE)
by Primary SOC and PT - Safety population

Primary System

| Organ Class | | | RIPK1 Inhibitor |
|---|---|---|---|
| Preferred | Placebo | | 600 mg |
| Term n(%) | (N = 20) | | (N = 47) |
| Any class | 3 | (15.0) | 6 (12.8) |
| INFECTIONS AND INFESTATIONS | 1 | (5.0) | 3 (6.4) |
| Bacterial infection | 1 | (5.0) | 1 (2.1) |
| Pneumonia bacterial | 0 | | 1 (2.1) |
| Pseudomonas infection | 0 | | 1 (2.1) |
| CARDIAC DISORDERS | 1 | (5.0) | 0 |
| Cardiac arrest | 1 | (5.0) | 0 |
| VASCULAR DISORDERS | 0 | | 1 (2.1) |
| Peripheral artery thrombosis | 0 | | 1 (2.1) |
| RESPIRATORY, THORACIC AND | 1 | (5.0) | 1 (2.1) |
| MEDIASTINAL DISORDERS | | | |
| Pulmonary embolism | 0 | | 1 (2.1) |
| Respiratory failure | 1 | (5.0) | 0 |
| GENERAL DISORDERS AND | 2 | (10.0) | 2 (4.3) |
| ADMINISTRATION SITE | | | |
| CONDITIONS | | | |
| Condition aggravated | 2 | (10.0) | 2 (4.3) |

SOC: System organ class, PT: Preferred term; MedDRA 23.1; n (%) = number and percentage of participants with at least one SAE.
Note:
Table sorted by SOC internationally agreed order and by decreasing frequency of PT in RIPK1 Inhibitor group. An adverse event is considered as treatment emergent if it occurred at the time from first dose of study intervention up to and including the day of last dose of study intervention plus 5 days.

4.3.3. Adverse Events Leading to Treatment Discontinuation

Overall, 6 TEAEs leading to treatment discontinuation were reported during the study in 5 participants.

One TEAE leading to treatment discontinuation was reported in 1 participant in the placebo group (alanine aminotransferase increased).

In the RIPK1 Inhibitor group, 5 TEAEs leading to treatment discontinuation were reported in 4 participants, 2 in one participant (arterial injury and peripheral artery thrombosis), 1 in one participant (Pseudomonas infection), 1 in one participant (condition aggravated), and 1 in one participant (condition aggravated).

4.3.4. Adverse Events of Special Interest

A table summarizing the number of participants with treatment emergent AESI by AESI category and PT is provided in Table 34.

Overall, 11 AESIs were reported during the study.

In the placebo group, 5 AESIs were reported in 3 participants, 1 in one participant (ALT increased, related to the IMP, recovered), 1 in one participant (ALT increased, recovered), and 3 in one participant (2 events of anemia, not recovered, and transaminases increased, recovered). Except for the AESI reported in one participant, all of these AESIs were considered as not IMP-related by Investigator.

In the RIPK1 Inhibitor group, 6 AESIs were reported in 6 participants: 1 in one participant (ALT increased, recovered), 1 in one participant (ALT increased, recovered), 1 in one participant (ALT increased, recovered), 1 in one participant (ALT increased, recovered), 1 in one participant (ALT increased, recovered), and 1 in one participant (ALT increased, recovered). All of these AESIs were considered as not IMP-related by Investigator.

Among these cases, ALT increased in one participant led to treatment discontinuation, and none of these cases were considered as SAE.

TABLE 34
Number (%) of participants with TEAE(s) (AESI)
by Primary SOC and PT - Safety population

Primary System

| Organ Class | | | RIPK1 Inhibitor |
|---|---|---|---|
| Preferred | Placebo | | 600 mg |
| Term n(%) | (N = 20) | | (N = 47) |
| Any class | 3 | (15.0) | 6 (12.8) |
| BLOOD AND LYMPHATIC SYSTEM | 1 | (5.0) | 0 |
| DISORDERS | | | |
| Anaemia | 1 | (5.0) | 0 |
| INVESTIGATIONS | 3 | (15.0) | 6 (12.8) |
| Alanine aminotransferase increased | 2 | (10.0) | 6 (12.8) |
| Transaminases increased | 1 | (5.0) | 0 |

AESI: AE of special interest,
SOC: System organ class,
PT: Preferred term
MedDRA 23.1; n (%) = number and percentage of participants with at least one AESI.
Note:
Table sorted by SOC internationally agreed order and by decreasing frequency of PT in RIPK1 Inhibitor group. An adverse event is considered as treatment emergent if it occurred at the time from first dose of study intervention up to and including the day of last dose of study intervention plus 5 days.

4.4. Clinical Laboratory Evaluations

4.4.1. White blood cells

4.4.1.1. Laboratory value over time

No clinically significant change in the mean WBC parameters (leukocytes, lymphocytes, neutrophils, eosinophils and basophils count) over time was observed. For change from baseline in WBC count, differential blood lymphocytes, neutrophil/lymphocyte ratio as markers of inflammation related to COVID-19 in the efficacy population, see Section 3.2.2.1.

4.4.1.2. Individual Participant Changes

Overall, post-baseline PCSAs for hematology parameters/white blood cells were observed in a small percentage of participants during the TEAE period, with little difference observed between the two treatment groups. The most frequently reported PCSAs are in monocytes (Table 35).

4.4.1.3. Individual Clinically Relevant Abnormalities

No participants had abnormal WBC parameters while on treatment that were considered as TEAEs.

TABLE 35
White blood cells - Number of participants with
abnormalities (PCSA) during the TEAE period according
to baseline status - safety population

| | | | RIPK1 Inhibitor | |
|---|---|---|---|---|
| | Placebo | | 600 mg | |
| | (N = 20) | | (N = 47) | |
| Laboratory parameter | Nor. | Abn. | Nor. | Abn. |
| PCSA criteria n/N1 | Bas. | Bas. | Bas. | Bas. |
| White blood cell count | | | | |
| <3 * 10\hat{ }9/L (Non-Black); <2 * | 0/12 | 0/8 | 1/37 | 0/10 |
| 10\hat{ }9/L (Black) | | | | |
| ≥16 * 10\hat{ }9/L | 1/12 | 2/8 | 2/37 | 1/10 |
| Neutrophils | | | | |
| <1.5 * 10\hat{ }9/L (Non-Black); <1 | 0/11 | 0/3 | 2/21 | 0/8 |
| * 10\hat{ }9/L (Black) | | | | |
| Lymphocytes | | | | |
| >4 * 10\hat{ }9/L | 1/7 | 0/7 | 3/18 | 2/11 |
| Monocytes | | | | |
| >0.7 * 10\hat{ }9/L | 5/8 | 1/6 | 12/19 | 6/10 |
| Basophils | | | | |
| >0.1 * 10\hat{ }9/L | 3/13 | 0/1 | 3/29 | 0/0 |
| Eosinophils | | | | |
| >0.5 * 10\hat{ }9/L or >ULN (if | 0/10 | 0/4 | 1/20 | 0/9 |
| ULN ≥ 0.5 * 10\hat{ }9/L) | | | | |

TEAE: Treatment emergent adverse event, PCSA: Potentially clinically significant abnormalities (Version of 2014-05-24 v1.0)
LLN/ULN: Lower/Upper Limit of Normal range,
Nor. Bas.: Normal baseline,
Abn. Bas.: Abnormal baseline (LLN/ULN or PCSA)
n/N1 = Number of participants who met the criterion at least once/ number of participants within each group who had that parameter assessed
Note:
A PCSA is considered to be during the TEAE period if it occurred at the time from first dose of study intervention up to and including the day of last dose of study intervention plus 5 days.
For eosinophils, values <LLN (or LLN missing) are counted as normal.

4.4.2. Red Blood Cells

4.4.2.1. Laboratory Value Over Time

There was no difference in the red blood cells (RBCs) parameters between the two treatment groups overtime during the on-treatment period.

4.4.2.2. Individual Participant Changes

Overall, post-baseline PCSAs for hematology parameters/RBCs were observed in a small percentage of participants during the TEAE period, with little difference observed between the two treatment groups. The most frequently reported PCSAs are in hematocrits (Table 36).

4.4.2.3. Individual Clinically Relevant Abnormalities

Three participants (2 in the placebo arm, 1 in the RIPK1 Inhibitor arm) reported PCSAs in hemoglobin and hematocrits parameters that were considered as TEAEs of anemia (Table 31). One of the three anemia events was reported as an AESI, in one participant in the placebo group. This participant died due to worsening of COVID-19 pneumonia. None of the other abnormal values in metabolic parameters are considered to require further description.

TABLE 36
Red blood cells, platelets and coagulation - Number of
participants with abnormalities (PCSA) during the TEAE
period according to baseline status - safety population

| | | | RIPK1 Inhibitor | |
|---|---|---|---|---|
| | Placebo | | 600 mg | |
| | (N = 20) | | (N = 47) | |
| Laboratory parameter | Nor. | Abn. | Nor. | Abn. |
| PCSA criteria n/N1 | Bas. | Bas. | Bas. | Bas. |
| Hemoglobin | | | | |
| ≤115 g/L (Male); ≤95 g/L (Female) | 2/15 | 2/5 | 1/29 | 4/18 |
| ≥185 g/L (Male); ≥165 g/L (Female) | 0/15 | 0/5 | 0/29 | 0/18 |
| Decrease from baseline ≥20 g/L | 3/20 | na | 4/47 | na |
| Hematocrit | | | | |
| ≤0.37 v/v (Male); ≤0.32 v/v (Female) | 5/14 | 2/6 | 4/30 | 11/17 |
| ≥0.55 v/v (Male); ≥0.5 v/v (Female) | 0/14 | 0/6 | 0/30 | 0/17 |
| Erythrocyte Count (RBC) | | | | |
| ≥6 * 10\hat{ }12/L | 1/15 | 0/5 | 0/30 | 1/17 |
| Platelet Count | | | | |
| <100 * 10\hat{ }9/L | 0/16 | 0/4 | 0/36 | 1/11 |
| ≥700 * 10\hat{ }9/L | 0/16 | 1/4 | 0/36 | 1/11 |

TEAE: Treatment emergent adverse event,
PCSA: Potentially clinically significant abnormalities
LLN/ULN: Lower/Upper Limit of Normal range,
Nor. Bas.: Normal baseline,
Abn. Bas.: Abnormal baseline (LLN/ULN or PCSA), na: not applicable
n/N1 = Number of participants who met the criterion at least once/number of participants within each group who had that parameter assessed
Note:
A PCSA is considered to be during the TEAE period if it occurred at the time from first dose of study intervention up to and including the day of last dose of study intervention plus 5 days.
For hemoglobin criterion on change from baseline, baseline values <LLN or >ULN (or LLN/ULN missing) are counted in one unique group (i.e. as normal).

4.4.3. Electrolytes

4.4.3.1. Laboratory Value Over Time

Descriptive statistics of laboratory values over time for electrolytes were not provided.

4.4.3.2. Individual Participant Changes

Overall, post-baseline PCSAs for electrolyte parameters were observed in a small percentage of participants during the TEAE period, with little difference observed between the two treatment groups (Table 37).

4.4.3.3. Individual Clinically Relevant Abnormalities

No participants had abnormal electrolyte parameters while on treatment that were considered as TEAEs.

TABLE 37
Electrolytes - Number of participants with abnormalities
(PCSA) during the TEAE period according to
baseline status - safety population

| | |
|---|---|
| | RIPK1 Inhibitor |
| Placebo | 600 mg |
| (N = 20) | (N = 47) |

| Laboratory parameter | Nor. | Abn. | Nor. | Abn. |
|---|---|---|---|---|
| PCSA criteria n/N1 | Bas. | Bas. | Bas. | Bas. |
| Sodium | | | | |

| | | | | | |
|---|---|---|---|---|---|
| ≤129 | mmol/L | 1/17 | 1/3 | 1/39 | 0/8 |
| ≥160 | mmol/L | 0/17 | 0/3 | 0/39 | 0/8 |

Potassium

| | | | | | |
|---|---|---|---|---|---|
| <3 | mmol/L | 0/18 | 0/2 | 0/40 | 0/7 |
| ≥5.5 | mmol/L | 2/18 | 1/2 | 4/40 | 1/7 |

TEAE: Treatment emergent adverse event,
PCSA: Potentially clinically significant abnormalities
LLN/ULN: Lower/Upper Limit of Normal range,
Nor. Bas.: Normal baseline,
Abn. Bas.: Abnormal baseline (LLN/ULN or PCSA)
n/N1 = Number of participants who met the criterion at least once/ number of participants within each group who had that parameter assessed
Note:
A PCSA is considered to be during the TEAE period if it occurred at the time from first dose of study intervention up to and including the day of last dose of study intervention plus 5 days.

4.4.4. Metabolic Function

4.4.4.1. Laboratory Value Over Time

Descriptive statistics of laboratory values over time for metabolic function parameter were not provided.

4.4.4.2. Individual Participant Changes

Overall, post-baseline PCSAs for metabolic parameters were observed in a small percentage of participants during the TEAE period, with little difference observed between the two treatment groups. The most frequently reported PCSAs in participant with a normal baseline are in glucose values (Table 38).

4.4.4.3. Individual Clinically Relevant Abnormalities

One participant in the RIPK1 Inhibitor arm with PCSAs of elevated glucose levels (from an abnormal baseline) that was considered as a TEAE of hyperglycemia. None of the other abnormal values in metabolic parameters are considered to require further description.

TABLE 38
Metabolism - Number of participants with abnormalities (PCSA)
during the TEAE period according to baseline status - safety population

| | Placebo | | | RIPK1 Inhibitor | | |
|---|---|---|---|---|---|---|
| | (N = 20) | | | 600 mg (N = 47) | | |
| Laboratory parameter | Nor. | Abn. | Mis. | Nor. | Abn. | Mis. |
| PCSA criteria n/N1 | Bas. | Bas. | Bas. | Bas. | Bas. | Bas. |

Glucose

| | | | | | | |
|---|---|---|---|---|---|---|
| ≤3.9 mmol/L and <LLN | 0/8 | 1/10 | 0/1 | 1/10 | 1/33 | 0/3 |
| ≥11.1 mmol/L (unfasted);≥ | 2/8 | 7/10 | 0/1 | 5/10 | 18/33 | 3/3 |
| 7 mmol/L (fasted) | | | | | | |

Albumin

| | | | | | | |
|---|---|---|---|---|---|---|
| ≤25 g/L | 1/10 | 2/9 | 1/1 | 0/18 | 0/28 | 0/0 |
| C-Reactive Protein | | | | | | |
| >2 ULN or >10 mg/L | 0/0 | 19/20 | 0/0 | 0/1 | 42/46 | 0/0 |
| (if ULN not provided) | | | | | | |

TEAE: Treatment emergent adverse event, PCSA: Potentially clinically significant abnormalities (Version of 2014 May 24 v1.0)
LLN/ULN: Lower/Upper Limit of Normal range, Nor. Bas.: Normal baseline, Abn. Bas.: Abnormal baseline (LLN/ULN or PCSA)
n/N1 = Number of participants who met the criterion at least once/number of participants within each group who had that parameter assessed
Note:
A PCSA is considered to be during the TEAE period if it occurred at the time from first dose of study intervention up to and including the day of last dose of study intervention plus 5 days.

4.4.5. Renal Function

4.4.5.1. Laboratory Value Over Time

Descriptive statistics for renal function parameters and summary plot showed no clinically meaningful changes during the TEAE period.

4.4.5.2. Individual Participant Changes

Overall, a small number of post-baseline PCSAs in renal parameters (creatinine and creatinine clearance) was observed during the TEAE period, with slightly higher occurrence rate in the placebo arms.

4.4.5.3. Individual Clinically Relevant Abnormalities

One participant in the placebo arm had abnormal renal function parameters that was reported as a TEAE of renal impairment. None of the other abnormal values in renal parameters are considered to require further description.

TABLE 39
Renal Function - Number of participants with abnormalities
(PCSA) during the TEAE period according to baseline
status - safety population

| | | | RIPK1 Inhibitor | |
|---|---|---|---|---|
| | Placebo | | 600 mg | |
| | (N = 20) | | (N = 47) | |
| Laboratory parameter | Nor. | Abn. | Nor. | Abn. |
| PCSA criteria n/N1 | Bas. | Bas. | Bas. | Bas. |
| Creatinine | | | | |
| ≥150 μmol/L (Adults) | 1/18 | 0/1 | 0/39 | 1/8 |
| ≥30% change from baseline | 3/19 | na | 3/47 | Na |
| ≥100% change from baseline | 1/19 | na | 0/47 | na |
| Creatinine Clearance (CG) | | | | |
| <15 mL/min (end stage renal disease) | 0/12 | 1/7 | 0/29 | 0/18 |
| ≥15-<30 mL/min (severe | 0/12 | 0/7 | 0/29 | 0/18 |
| decrease in GFR) | | | | |
| ≥30-<60 mL/min (moderate | 0/12 | 0/7 | 0/29 | 4/18 |
| decrease in GFR) | | | | |
| ≥60-<90 mL/min (mild | 0/12 | 5/7 | 5/29 | 11/18 |
| decrease in GFR) | | | | |

TEAE: Treatment emergent adverse event,
PCSA: Potentially clinically significant abnormalities
LLN/ULN: Lower/Upper Limit of Normal range,
Nor. Bas.: Normal baseline,
Abn. Bas.: Abnormal baseline (LLN/ULN or PCSA)
n/N1 = Number of participants who met the criterion at least once/number of participants within each group who had that parameter assessed
Note:
A PCSA is considered to be during the TEAE period if it occurred at the time from first dose of study intervention up to and including the day of last dose of study intervention plus 5 days.
For creatinine criterion on % change from baseline, baseline values <LLN or >ULN (or LLN/ULN missing) are counted in one unique group (i.e. as normal).

4.4.6. Hepatic Parameters

4.4.6.1. Individual Participant Changes

Overall, a small number of post-baseline PCSAs in liver function parameters was observed during the TEAE period (Table 40). No participants reported any combined PCSAs for liver function. The most frequently reported PCSA was elevated ALT.

Sixteen participants had ALT>3 ULN (7 in the placebo group and 9 in the RIPK1 Inhibitor group). Three participants had ALT>5 ULN (2 in the placebo group and 1 in the RIPK1 Inhibitor group). One participant had ALT>10 ULN in the placebo group.

Five participants had PCSAs of AST>3 ULN (3 in the placebo group and 2 in the RIPK1 Inhibitor group). Three participants in AST>5 ULN (2 in the placebo group and 1 in the RIPK1 Inhibitor group). Four participants in alkaline phosphatase>1.5 ULN (2 in the placebo group and 2 in the RIPK1 Inhibitor group). One participant in total bilirubin>1.5 ULN in the RIPK1 Inhibitor group.

4.4.6.2. Individual Clinically Relevant Abnormalities

Six participants in the RIPK1 Inhibitor arm, and 3 participants om the placebo arm had abnormal ALT levels while on treatment that were considered as AESIs of ALT increased.

One participant in the placebo arm had abnormal ALT and AST levels while on treatment that were considered as AESIs of transaminase increase. One participant in the RIPK1 Inhibitor arm had abnormal ALT and AST levels that were considered as a post-treatment AESIs of transaminase increase. These two participants had fatal outcome due to worsening of COVID-19.

Further information is provided in Section 4.3.4.

TABLE 40
Liver Function - Number of participants with abnormalities (PCSA) during
the TEAE period according to baseline status - safety population

| | Placebo | | | RIPK1 Inhibitor 600 mg | | |
|---|---|---|---|---|---|---|
| | (N = 20) | | | (N = 47) | | |
| Laboratory parameter | Nor. | Abn. | Mis. | Nor. | Abn. | Mis. |
| PCSA criteria n/N1 | Bas. | Bas. | Bas. | Bas. | Bas. | Bas. |

Alanine Aminotransferase (ALT)

| | | | | | | |
|---|---|---|---|---|---|---|
| >3 ULN | 2/11 | 5/9 | 0/0 | 2/27 | 7/20 | 0/0 |
| >5 ULN | 2/11 | 0/9 | 0/0 | 1/27 | 0/20 | 0/0 |
| >10 ULN | 1/11 | 0/9 | 0/0 | 0/27 | 0/20 | 0/0 |
| >20 ULN | 0/11 | 0/9 | 0/0 | 0/27 | 0/20 | 0/0 |

Aspartate Aminotransferase (AST)

| | | | | | | |
|---|---|---|---|---|---|---|
| >3 ULN | 0/12 | 3/8 | 0/0 | 1/23 | 1/24 | 0/0 |
| >5 ULN | 0/12 | 2/8 | 0/0 | 1/23 | 0/24 | 0/0 |
| >10 ULN | 0/12 | 0/8 | 0/0 | 0/23 | 0/24 | 0/0 |

Alkaline Phosphatase

| | | | | | | |
|---|---|---|---|---|---|---|
| >1.5 ULN | 0/15 | 2/4 | 0/1 | 2/46 | 0/0 | 0/0 |
| Total Bilirubin | | | | | | |
| >1.5 ULN | 0/18 | 0/2 | 0/0 | 1/45 | 0/2 | 0/0 |
| >2 ULN | 0/18 | 0/2 | 0/0 | 0/45 | 0/2 | 0/0 |

Conjugated bilirubin

| | | | | | | |
|---|---|---|---|---|---|---|
| >35% Bilirubin and Bilirubin> | 0/20 | 0/0 | 0/0 | 0/46 | 0/0 | 0/1 |
| 1.5 ULN | | | | | | |

TEAE: Treatment emergent adverse event, PCSA: Potentially clinically significant abnormalities
LLN/ULN: Lower/Upper Limit of Normal range, Nor. Bas.: Normal baseline, Abn. Bas.: Abnormal baseline (LLN/ULN or PCSA), Mis. Bas.: Missing baseline
n/N1 = Number of participants who met the criterion at least once/number of participants within each group who had that parameter assessed
Note:
A PCSA is considered to be during the TEAE period if it occurred at the time from first dose of study intervention up to and including the day of last dose of study intervention plus 5 days.
For ALT, AST, ALP and Total Bilirubin, values <LLN (or LLN missing) are counted as normal.

4.5. Vital Signs, Physical Findings, and Other Safety Observations

4.5.1. Vital Signs

4.5.1.1. Vital Sign Values Over Time

No clinically meaningful changes from baseline throughout the course of the study was observed in vital signs parameters, including blood pressure, temperature, heart rate, and respiratory rate.

4.5.1.2. Individual Participant Changes

Overall, the number of participants with post-baseline PCSAs for vital signs during the TEAE period was low and in both treatment arms. The most often observed PCSA was systolic blood pressure≤95 mmHg and decrease from baseline≥20 mmHg, observed in 4 participants in the RIPK1 Inhibitor group and 3 participants in the placebo group (Table 41).

TABLE 41
Vital signs - Number of participants with abnormalities
(PCSA) during the TEAE period - Safety population

| | | RIPK1 Inhibitor |
|---|---|---|
| Vital signs parameter | Placebo | 600 mg |
| PCSA criteria n/N1 | (N = 20) | (N = 47) |
| Diastolic Blood Pressure | | |
| ≤45 mmHg and decrease from | 1/20 | 1/47 |
| baseline ≥10 mmHg | | |
| ≥110 mmHg and increase from | 0/20 | 0/47 |
| baseline ≥10 mmHg | | |
| Heart Rate | | |
| ≤50 beats/min and decrease from | 1/20 | 0/47 |
| baseline ≥20 beats/min | | |
| ≥120 beats/min and increase from | 3/20 | 1/47 |
| baseline 20 beats/min | | |
| Systolic Blood Pressure | | |
| ≤95 mmHg and decrease from | 3/20 | 4/47 |
| baseline ≥20 mmHg | | |
| ≥160 mmHg and increase from | 1/20 | 4/47 |
| baseline ≥20 mmHg | | |

PCSA: Potentially clinically significant abnormalities (Version of 2014-05-24 v1.0) n/N1 = Number of participants who met the criterion at least once/ number of participants within each group who had that parameter assessed
Note:
A PCSA is considered to be during the TEAE period if it occurred from the time of first dose of study drug up to and including the day of last dose of study drug plus 5 days

4.5.1.3. Individual Clinically Relevant Abnormalities

No participants had abnormalities in vital sign parameters while on treatment that were reported as adverse events.

4.5.2. Electrocardiograms

4.5.2.1. Individual Participant Changes

The most frequently reported ECG PCSAs included:
Heart rate>90 beats/min was observed in 11 participants (5 in the placebo group and 6 in the RIPK1 Inhibitor group).
   In additional, 7 participants reported heart rate>90 beats/min and increase from baseline≥20 beats/min (2 in the placebo group and 5 in the RIPK1 Inhibitor group).
QRS interval>110 ms was observed in 7 participants (1 in the placebo group and 6 in the RIPK1 Inhibitor group).
QTc Bazett (QTcB)>450 ms was observed in 8 participants (3 in the placebo group and 5 in the RIPK1 Inhibitor group).
   Additionally, 4 participants reported QTc Bazett>480 msec (1 in the placebo group and 3 in the RIPK1 Inhibitor group) and 3 participants reported QTc Bazett>500 ms in the RIPK1 Inhibitor group.
QTc Bazett—change from baseline>60 ms was observed in 5 participants in the RIPK1 Inhibitor group.

All other PCSAs related to the ECG parameters were observed in 3 participants or less for each treatment.

A listing of ECG data for participants with QTcB/F>480 ms and/or delta QTcB/F>60 ms is provided in Table 46.

4.5.2.2. Individual Clinically Relevant Abnormalities

No participants had abnormalities in ECG parameters while on treatment that were reported as adverse events.

TABLE 42
ECG - Number of participants with abnormalities
(PCSA) during the TEAE period - safety population

| | | | RIPK1 Inhibitor |
|---|---|---|---|
| ECG parameter | | Placebo | 600 mg |
| PCSA criteria n/N1 | | (N = 20) | (N = 47) |
| Heart Rate | | | |
| <50 | beats/min | 0/19 | 1/44 |
| <50 beats/min and decrease from | | 0/19 | 0/44 |
| baseline ≥20 beats/min | | | |
| <40 | beats/min | 0/19 | 0/44 |
| >90 | beats/min | 5/19 | 6/44 |
| >90 beats/min and increase from | | 2/19 | 5/44 |
| baseline ≥20 beats/min | | | |
| >100 | beats/min | 3/19 | 3/44 |
| >100 beats/min and increase from | | 2/19 | 3/44 |
| baseline ≥20 beats/min | | | |
| >120 | beats/min | 1/19 | 1/44 |
| >120 beats/min and increase from | | 1/19 | 1/44 |
| baseline 20 beats/min | | | |
| PR Interval | | | |
| >200 | msec | 0/18 | 1/43 |
| >200 msec and increase from | | 0/18 | 0/43 |
| baseline ≥25% | | | |
| >220 | msec | 0/18 | 1/43 |
| >220 msec and increase from | | 0/18 | 0/43 |
| baseline ≥25% | | | |
| >240 | msec | 0/18 | 0/43 |
| QRS Interval | | | |
| >110 | msec | 1/19 | 6/44 |
| >110 msec and increase from | | 0/19 | 3/44 |
| baseline ≥25% | | | |
| >120 | msec | 0/19 | 3/44 |
| >120 msec and increase from | | 0/19 | 2/44 |
| baseline ≥25% | | | |
| QT Interval | | | |
| >500 | msec | 0/19 | 0/44 |
| QTc Bazett | | | |
| >450 | msec | 3/19 | 5/44 |
| >480 | msec | 1/19 | 3/44 |
| >500 | msec | 0/19 | 3/44 |
| QTc Bazett - change from baseline | | | |
| Increase from baseline ]30-60] msec | | 0/18 | 2/39 |
| Increase from baseline >60 msec | | 0/18 | 5/39 |
| QTc Fridericia | | | |
| >450 | msec | 0/13 | 1/29 |
| >480 | msec | 0/13 | 1/29 |
| >500 | msec | 0/13 | 0/29 |

| | | |
|---|---|---|
| QTc Fridericia - change from baseline | | |
| Increase from baseline ]30-60] msec | 0/12 | 2/25 |
| Increase from baseline >60 msec | 0/12 | 2/25 |

PCSA: Potentially clinically significant abnormalities (Version of 2014-05-24 v1.0)
n/N1 = Number of participants who met the criterion at least once/number of participants within each group who had that parameter assessed
Note:
A PCSA is considered to be during the TEAE period if it occurred from the time of first dose of study drug up to and including the day of last dose of study drug plus 5 days

4.6. Safety Conclusions

Overall, 34 (50.7%) of 67 participants experienced at least one TEAE during the study (10 out of 20 participants in the placebo group and 24 out of 47 participants in the RIPK1 Inhibitor group). The percentage of participants with any TEAEs was balanced between the placebo (50.0%) and active treatment (51.1%) arms.

There were 4 deaths overall during the conduct of the study up to Day 28 due to worsening of COVID-19 disease with 2 participants in the placebo group (10.0%) and 2 participants in the RIPK1 Inhibitor group (4.3%).

Treatment-emergent SAEs were reported in 3 out of 20 (15.0%) participants in the placebo group and 6 out of 47 (12.8%) participants in the RIPK1 Inhibitor group, deemed as not related to IMP by the Investigators.

Treatment-emergent AE leading to permanent study treatment discontinuation were reported in 1 out of 20 (5.0%) participants in the placebo group and 4 out of 47 (8.5%) participants in the RIPK1 Inhibitor group.

Adverse events of special interest were reported in 3 out of 20 (15.0%) participants in the placebo group and 6 out of 47 (12.8%) participants in the RIPK1 Inhibitor group. AESI and SAEs were assessed as correlated to COVID-19 associated signs, symptoms and/or complications.

In the RIPK1 Inhibitor group, the most frequently reported TEAE by PT was alanine aminotransferase increased, which were mainly reversible increases in ALT deemed as not related to IMP by the Pis. There was also no relevant difference between patients administered with placebo and RIPK1 Inhibitor in occurrence of any PCSAs for liver function parameters.

### 5. PHARMACOKINETIC EVALUATION

5.1. Plasma Concentrations

RIPK1 Inhibitor concentrations were below limit of quantitation (BLOC) in the placebo except for one participant, with plasma concentration of 1530 ng/mL on Day 1 and 2300 ng/mL on Day 3, for this participant intubated who received the treatment as a suspension via the feeding tube, there was a suspicion of treatment inversion with another patient included in the same site on the same day randomized in the verum group but with plasma concentration BLOQ. A secondary analysis on the primary pharmacodynamics endpoint was conducted without these two subjects; and one participant, with 1 plasma concentration of 1460 ng/mL on Day 4 (day of discharge) whereas previous samples on Day 1 and Day 3 were found BLOQ. No explanation has been found.

5.2. Pharmacokinetic Parameters

The pharmacokinetic parameters in participants with severe COVID-19 were assessed by Bayesian analysis using a POP population PK model (P0H0757) developed in other Phase 1 studies.

PK parameters were determined for 46 participants (one participant was excluded because all plasma concentrations were BLOQ). A summary of descriptive statistics on RIPK1 Inhibitor plasma AUC₀₋₁₂, Cₘₐₓ, and C_{trough} over 2 weeks of treatment are presented in Table 43.

TABLE 43
Mean (SD) RIPK1 Inhibitor AUC_{0-12 h}, Cₘₐₓ and C_{trough}

| | AUC₀₋₁₂ | Cₘₐₓ | C_{trough} |
|---|---|---|---|
| | (ng · h/mL) | (ng/mL) | (ng/mL) |
| Day 1 (n = 46) | 28224 (5180) | 3681 (720) | 1457 (442) |
| Day 3 (n = 42) | 42214 (10949) | 5169 (1056) | 2025 (783) |
| Day 7 (n = 26) | 43797 (11314) | 5336 (1069) | 2142 (838) |
| Day 14 (n = 10) | 48352 (12683) | 5634 (1234) | 2524 (875) |

In participants with severe COVID-19, after administration of RIPK1 Inhibitor 300 mg BID for up to 14 days, steady state was reached on Day 3. RIPK1 Inhibitor plasma exposure was similar as those predicted from PK profiles observed in healthy participants. Among the 46 participants, only one participant received RIPK1 Inhibitor as a suspension by feeding tube, exposure parameters observed for this participant were in the range of those observed for the other participants.

No obvious exposure difference between male and female was observed. Some trends of exposure decrease with increasing weight (14% higher AUC₀₋₁₂ₕ in patients<85.6 kg as compared to ≥85.6 kg) were observed.

5.3. Pharmacokinetic Conclusions

In participants with severe COVID-19, after administration of RIPK1 Inhibitor 300 mg BID for up to 14 days, RIPK1 Inhibitor plasma exposure was similar as those predicted from PK profiles observed in healthy volunteers. Steady state was reached on Day 3 with mean (SD) values of 2025 (783) ng/mL for C_{trough}, 5169 (1056) ng/mL for Cₘₐₓ and 42214 (10949) ng·h/mL for AUC₀₋₁₂ₕ.

### 6. ADDITIONAL DATA

TABLE 44
Overview of adverse event profile: Pre-treatment
emergent adverse events - Safety population

| | | RIPK1 Inhibitor |
|---|---|---|
| | Placebo | 600 mg |
| n (%) | (N = 20) | (N = 47) |
| Participants with | 0 | 2 (4.3) |
| any pre-treatment AE | | |
| Participants with | 0 | 1 (2.1) |
| severe pre-treatment AE | | |
| Participants with | 0 | 1 (2.1) |
| any pre-treatment SAE | | |
| Participants with | 0 | 0 |
| any pre-treatment AE leading to death | | |

AE: Adverse event,
SAE: Serious adverse event
n (%) = number and percentage of participants with at least one pre-treatment AE

TABLE 45
Overview of adverse event profile: Post-treatment
emergent adverse events - Safety population

| | | RIPK1 Inhibitor |
|---|---|---|
| | Placebo | 600 mg |
| n (%) | (N = 20) | (N = 47) |
| Participants with | 2 (10.0) | 6 (12.8) |
| any post-treatment AE | | |
| Participants with | 1 (5.0) | 1 (2.1) |
| severe post-treatment AE | | |
| Participants with any | 1 (5.0) | 1 (2.1) |
| post-treatment SAE | | |
| Participants with any | 1 (5.0) | 1 (2.1) |
| post-treatment AE leading to death | | |
| Participants with any | 0 | 0 |
| post-treatment related to the compound | | |

AE: Adverse event,
SAE: Serious adverse event
n (%) = number and percentage of participants with at least one post-treatment AE
Note:
Post-treatment Aes are defined as Aes that developed or worsened or became serious during the post-treatment period

TABLE 46
Listing of participants with QTcB/F > 480 ms and/or delta QTcB/F > 60 ms - safety population

| | | | | | PR (ms) | | QRS (ms) | | | | | | | |
|---|---|---|---|---|---|---|---|---|---|---|---|---|---|---|
| | Examination | | HR (bpm) | | | % | | % | QT (ms) | | QTcB (ms) | | QTcF (ms) | |
| Visit | Date | Time | Value | Delta | Value | change | Value | change | Value | Delta | Value | Delta | Value | Delta |

Treatment group = Placebo - Participant = (Male/47 years/170 cm/75.0 kg/26.0 kg/m²/White)

| | | | | | | | | | | | | | | |
|---|---|---|---|---|---|---|---|---|---|---|---|---|---|---|
| Baseline | 2020 Jul. 27 | 14:25 | 96 B + | 0 + | 140 B | 0.0 | 100 B | 0.0 | 380 B | 0 | 480 B + | 0 | 444 B | 0 |
| Dis- | 2020 Aug. 03 | 10:58 | 80 | −16 | 180 | 28.6 | 110 | 10.0 | 360 | −20 | 487 ++ | 7 | 440 | −4 |
| charge | | | | | | | | | | | | | | |
| Before | | | | | | | | | | | | | | |
| Day 15 | | | | | | | | | | | | | | |

Treatment group = RIPK1 Inhibitor 600 mg - Participant = (Male/71 years/182 cm/98.0 kg/29.6 kg/m²/White)

| | | | | | | | | | | | | | | |
|---|---|---|---|---|---|---|---|---|---|---|---|---|---|---|
| Baseline | 2020 Aug. 02 | 9:33 | 82 B | 0 | 146 B | 0.0 | 110 B | 0.0 | 374 B | 0 | 347 B | 0 | 415 B | 0 |
| Dis- | 2020 Aug. 13 | 14:39 | 81 | −1 | 164 | 12.3 | 95 | −14 | 371 | −3 | 431 | 84 ++ | 410 | −5 |
| charge | | | | | | | | | | | | | | |
| Before | | | | | | | | | | | | | | |
| Day 15 | | | | | | | | | | | | | | |

Treatment group = RIPK1 Inhibitor 600 mg - Participant = (Female/60 years/164 cm/114.0 kg/42.4 kg/m²/White)

| | | | | | | | | | | | | | | |
|---|---|---|---|---|---|---|---|---|---|---|---|---|---|---|
| Baseline | 2020 Jul. 17 | 20:00 | 58 B | 0 | 180 B | 0.0 | 100 B | 0.0 | 390 B | 0 | 383 B | 0 | 385 B | 0 |
| Dis- | 2020 Jul. 22 | 10:00 | 96 + | 38 + | 160 | −11 | 80 | −20 | 360 | −30 | 455 + | 72 ++ | 421 | 36 + |
| charge | | | | | | | | | | | | | | |
| Before | | | | | | | | | | | | | | |
| Day 15 | | | | | | | | | | | | | | |

Treatment group = RIPKl Inhibitor 600 mg - Participant = (Female/64 years/155 cm/70.0 kg/29.1 kg/m²/White)

| | | | | | | | | | | | | | | |
|---|---|---|---|---|---|---|---|---|---|---|---|---|---|---|
| Baseline | 2020 Sep. 15 | 10:51 | 101 B ++ | 0 ++ | 120 B | 0.0 | 160 B ++ | 0.0 ++ | 360 B | 0 | 467 B + | 0 | 420 B | 0 |
| Dis- | 2020 Sep. 17 | 10:00 | 76 | −25 | 120 | 0.0 | 100 | −38 | 460 | 100 | 517 ++ | 50 + | 495 ++ | 75 ++ |
| charge | | | | | | | | | | | | | | |
| Before | | | | | | | | | | | | | | |
| Day 15 | | | | | | | | | | | | | | |

Treatment group = RIPK1 Inhibitor 600 mg - Participant = (Male/49 years/179 cm/84.0 kg/26.2 kg/m²/White)

| | | | | | | | | | | | | | | |
|---|---|---|---|---|---|---|---|---|---|---|---|---|---|---|
| Baseline | 2020 Sep. 22 | 9:22 | 78 B | 0 | 120 B | 0.0 | 80 B | 0.0 | 380 B | 0 | 380 B | 0 | 414 B | 0 |
| Dis- | 2020 Sep. 28 | 19:00 | 73 | −5 | 120 | 0.0 | 84 | 5.0 | 380 | 0 | 449 | 69 ++ | 380 | −34 |
| charge | | | | | | | | | | | | | | |
| Before | | | | | | | | | | | | | | |
| Day 15 | | | | | | | | | | | | | | |

Treatment group = RIPKl Inhibitor 600 mg - Participant = (Male/60 years/176 cm/125.0 kg/40.4 kg/m²/White)

| | | | | | | | | | | | | |
|---|---|---|---|---|---|---|---|---|---|---|---|---|
| Baseline | 2020 Sep. 02 | 19:46 | 83 B | 0 | 160 B | 0.0 | 84 B | 0.0 | 348 B | 0 | 409 B | 0 |
| Dis- | 2020 Sep. 10 | 9:10 | 77 | −6 | 160 | 0.0 | 80 | −4.8 | 466 | 118 | 530 ++ | 121 ++ |
| charge | | | | | | | | | | | | |
| Before | | | | | | | | | | | | |
| Day 15 | | | | | | | | | | | | |

Treatment group = RIPKl Inhibitor 600 mg - Participant = (Female/65 years/164 cm/120.0 kg/44.6 kg/m²/White)

| | | | | | | | | | | | | |
|---|---|---|---|---|---|---|---|---|---|---|---|---|
| Baseline | 2020 Sep. 04 | 19:37 | 69 B | 0 | 94 B | 0.0 | 114 B + | 0.0 + | 478 B | 0 | 514 B ++ | 0 |
| Day 15 | 2020 Sep. 19 | 10:29 | 69 | 0 | 124 | 31.9 | 82 | −28 | 482 | 4 | 518 ++ | 4 |

Treatment group = RIPKl Inhibitor 600 mg - Participant = (Male/36 years/181 cm/87.2 kg/26.6 kg/m²/White)

| | | | | | | | | | | | | | | |
|---|---|---|---|---|---|---|---|---|---|---|---|---|---|---|
| Baseline | 2020 Aug. 19 | 12:42 | 68 B | 0 | 150 B | 0.0 | 80 B | 0.0 | 340 B | 0 | 362 B | 0 | 354 B | 0 |
| Dis- | 2020 Aug. 27 | 8:42 | 86 | 18 | 150 | 0.0 | 100 | 25.0 | 370 | 30 | 443 | 81 ++ | 417 | 63 ++ |
| charge | | | | | | | | | | | | | | |
| Before | | | | | | | | | | | | | | |
| Day 15 | | | | | | | | | | | | | | |

PCSA: Potentially clinically significant abnormalities
B: Baseline, Delta: Change from baseline (B), % change: Percent change from baseline (B), r: Rechecked value
−/−− or +/++: Abnormal value reaching the 1^{st}/2^{nd} lower or the 1^{st}/2^{nd} upper PCSA limit
Note:
Baseline is defined as the screening predose assessment value
Note:
A PCSA is considered to be during the TEAE period if it occurred from the time of first dose of study drug up to and including the day of last dose of study drug plus 5 days

### 7. DISCUSSION AND OVERALL CONCLUSIONS

The administration of daily doses of the RIPK1 Inhibitor over 15 days in 67 participants with severe COVID-19 (placebo: 20; RIPK1 Inhibitor: 47) was generally safe and well tolerated as compared to placebo, in combination with standard of care. There were 4 deaths during the conduct of the study up to Day 28 due to worsening of COVID-19 disease with 2 participants in the placebo group (10.0%) and 2 participants in the active group (4.3%).

There is no statistically significant difference in the primary endpoint of relative change in CRP at Day 7 from baseline between the treatment and the placebo groups (p-value: 0.302). However, the relative CRP decrease from baseline is numerically greater in the treatment group as indicated by the ratio of the geometric means of relative change from baseline with RIPK1 Inhibitor versus placebo on Day 7 that equals 0.85 [90% CI: 0.49 to 1.45]. A trend toward an earlier decrease in CRP is observed in the KM graph—the p-value on the difference between KM curves is nearing statistical significance with 0.0557. Of note, corticosteroids, which are known to decrease CRP levels, were administered as standard of care in approximately 65% of the participants in each treatment group. Consistent trends toward greater improvements in clinical endpoints were noted in the RIPK1 Inhibitor group as compared to the placebo group with quicker and larger increase of SpO₂/FiO₂, along with improvements in SpO₂, VFDs, RFFDs and in the 7-point clinical scale scores over the treatment period.

In participants with severe COVID-19, after administration of RIPK1 Inhibitor 300 mg BID for up to 14 days, RIPK1 Inhibitor plasma exposure was similar as those predicted from PK profiles observed in healthy volunteers. Steady state was reached on Day 3 with mean (SD) values of 2025 (783) ng/mL for C_{trough}, 5169 (1056) ng/mL for Cₘₐₓ and 42214 (10949) ng·h/mL for AUC₀₋₁₂ₕ.

### 8. REFERENCES

1. Zhu N, Zhang D, Wang W, Li X, Yang B, Song J, et al. A novel coronavirus from patients with pneumonia in China, 2019. N Engl J Med. 2020; 382(8):727-33.
2. Lau S K P, Lau C C Y, Chan K H, Li C P Y, Chen H, Jin D Y, et al. Delayed induction of proinflammatory cytokines and suppression of innate antiviral response by the novel Middle East respiratory syndrome coronavirus: implications for pathogenesis and treatment. J Gen Virol. 2013; 94(Pt12):2679-90.
3. Chen N, Zhou M, Dong X, Qu J, Gong F, Han Y, et al. Epidemiological and clinical characteristics of 99 cases of 2019 novel coronavirus pneumonia in Wuhan, China: a descriptive study. Lancet. 2020; 395(10223):507-13.
4. Huang C, Wang Y, Li X, Ren L, Zhao J, Hu Y, et al. Clinical features of patients infected with 2019 novel coronavirus in Wuhan, China. Lancet. 2020; 395(10223):497-506.
5. Wang D, Hu B, Hu C, Zhu F, Liu X, Zhang J, et al. Clinical characteristics of 138 hospitalized patients with 2019 novel coronavirus-infected pneumonia in Wuhan, China. JAMA. 2020; 323(11)1061-9.
6. Zhang Y, Li J, Zhan Y, Wu L, Yu X, Zhang W, et al. Analysis of serum cytokines in patients with severe acute respiratory syndrome. Infect Immun. 2004; 72(8):4410-5.
7. Huang K J, Su I J, Theron M, Wu Y C, Lai S K, Liu C C, et al. An interferon-gamma-related cytokine storm in SARS patients. J Med Virol. 2005; 75(2):185-94.
8. Tisoncik J R, Korth M J, Simmons C P, Farrar J, Martin T R, Katze M G. Into the eye of the cytokine storm. Microbiol Mol Biol Rev. 2012; 76(1):16-32.
9. Chien J Y, Hsueh P R, Cheng W C, Yu C J, Yang P C. Temporal changes in cytokine/chemokine profiles and pulmonary involvement in severe acute respiratory syndrome. Respirology (Carlton, Vic) 2006; 11:715-22.
10. Kim E S, Choe P G, Park W B, Oh H S, Kim E J, Nam E Y, et al. Clinical progression and cytokine profiles of Middle East respiratory syndrome coronavirus infection. J Korean Med Sci. 2016; 31(11):1717-25.
11. Wang W K, Chen S Y, Liu I J, Kao C L, Chen H L, Chiang B L, et al. Temporal relationship of viral load, ribavirin, interleukin (IL)-6, IL-8, and clinical progression in patients with severe acute respiratory syndrome. Clin Infect Dis. 2004; 39(7):1071-5.
12. Ackermann M, Verleden S E, Kuehnel M, Haverich A, Welte T, Laenger F, et al. Pulmonary vascular endothelialitis, thrombosis, and angiogenesis in COVID-19. N Engl J Med. 2020. Doi:10.1056/NEJMoa2015432. Online ahead of print.
13. Zelic M, Roderick J E, O'Donnell J A, Lehman J, Lim S E, Janardhan H P, et al. RIPK1-dependent endothelial necroptosis underlies systemic inflammatory response syndrome. J Clin Invest. 2018; 128(5):2064-75.
14. Takahashi N, Duprez L, Grootjans S, Cauwels A, Nerinckx W, DuHadaway J B, et al. Necrostatin-1 analogues: critical issues on the specificity, activity and in vivo use in experimental disease models. Cell Death Dis. 2012; 3(11):e437.
15. Duprez L, Takahashi N, Van Hauwermeiren F, Vandendriessche B, Goossens V, Vanden Berghe T, et al. RIP kinase-dependent necrosis drives lethal systemic inflammatory response syndrome. Immunity. 2011; 35(6):908-18.
16. Newton K, Dugger D L, Maltzman A, Greve J M, Hedehus M, Martin-McNulty B, et al. RIPK3 deficiency or catalytically inactive RIPK1 provides greater benefit than MLKL deficiency in mouse models of inflammation and tissue injury. Cell Death Differ. 2016; 23(9):1565-76.
17. Delvaeye T, De Smet M A J, Verwaerde S, Decrock E, Czekaj A, Vandenbroucke R E, et al. Blocking connexin43 hemichannels protects mice against tumour necrosis factor-induced inflammatory shock. Sci Rep. 2019; 9(1):16623.

What is claimed is:

1. A method of treating a subject at risk of or having Cytokine Release Syndrome (CRS), comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

2. A method of treating a subject in a hyperinflammatory state, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

3. A method of treating a subject at risk of or having Systemic Inflammatory Response Syndrome (SIRS), comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

4. A method of reducing inflammation in a subject at risk of or having CRS or SIRS, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

5. A method of reducing organ damage in a subject at risk of or having CRS or SIRS, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

6. A method of reducing sepsis-related inflammation and organ injury in a subject, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

7. A method of treating a subject having influenza-like illness, comprising administering to a subject in need thereof a RIPK1 inhibitor comprising (S)-5-benzyl-N-(5-methyl oxo-2,3,4,5-tetrahydropyrido[3,2-b][1,4]oxazepin-3-yl)-4H-1,2,4-triazole carboxamide, and/or a pharmaceutically acceptable salt, tautomer, stereoisomer or mixture of stereoisomers thereof.

8. A method of reducing symptoms related to coronavirus infection, comprising administering to a subject in need thereof a RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt, tautomer, stereoisomer or mixture of stereoisomers thereof.

9. The method of claim 8, wherein the coronavirus infection is by COVID-19/2019-nCoV/SARS-CoV-2, SARS-CoV, and/or MERS-CoV.

10. The method of any one of claims 1-9, wherein the RIPK1 inhibitor is (S)-5-benzyl-N-(5-methyl-4-oxo-2,3,4,5-tetrahydropyrido[3,2-b][1,4]oxazepin-3-yl)-4H-1,2,4-triazole-3-carboxamide, and/or a pharmaceutically acceptable salt thereof.

11. The method of any one of claims 1-10, wherein a dose of about 5 mg to about 1000 mg of the RIPK1 inhibitor is administered.

12. The method of claim 11, wherein the dose is 400 mg.

13. The method of claim 11, wherein the dose is 600 mg.

14. The method of claim 11, wherein the dose is 800 mg.

15. The method of claim 11, wherein the dose is 1000 mg.

16. The method of any one of claims 1-15, wherein the RIPK1 inhibitor is administered daily.

17. The method of any one of claims 1-16, wherein the RIPK1 inhibitor is administered in conjunction with antiviral therapy.

18. The method of claim 17, wherein the antiviral therapy is chosen from remdesivir, hydroxychloroquinine, galidesivir, oseltamivir, paramivir, zanamivir, ganciclovir, acyclovir, ribavirin, lopinavir, ritonavir, favipiravir, darunavir or a combination thereof.

19. The method of any one of claims 1-16, wherein the RIPK1 inhibitor is administered in conjunction with a corticosteroid treatment.

20. The method of claim 18, wherein the corticosteroid treatment is chosen from dexamethasone, betamethasone, prednisone, prednisolone, methylprednisolone, cortisone, hydrocortisone, triamcinolone, or ethamethasoneb or a combination thereof.

21. The method of any one of claims 1-20, wherein the RIPK1 inhibitor is administered orally.

22. The method of any one of claims 1-20, wherein the RIPK1 inhibitor is administered via gastric feeding tube.

23. The method of any one of claims 1-22, wherein the condition of the subject comprises a systemic hyperinflammatory response.

24. The method of claim 24, wherein the systemic hyperinflammatory response is shown by increase in CRP, decrease in leukocyte number, change in neutrophil number, decrease in neutrophil to lymphocyte ratio, and/or increase in IL-6.

25. The method of any one of claims 1-22, wherein the condition of the subject indicates innate immunity activation.

26. The method of claim 25, wherein innate immunity activation is shown by increase in CRP, change in neutrophil number, and/or increase in IL-6.

27. A RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in treating a subject at risk of or having Cytokine Release Syndrome (CRS) or Inflammatory Response Syndrome (SIRS).

28. A RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in treating a subject in a hyperinflammatory state.

29. A RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in reducing inflammation or organ damage in a subject at risk of or having CRS or SIRS.

30. A RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in reducing sepsis-related inflammation or organ damage in a subject.

31. A RIPK1 inhibitor comprising (S)-5-benzyl-N-(5-methyl-4-oxo-2,3,4,5-tetrahydropyrido[3,2-b][1,4]oxazepin-3-yl)-4H-1,2,4-triazole-3-carboxamide and/or a pharmaceutically acceptable salt, tautomer, stereoisomer or mixture of stereoisomers thereof for use in treating a subject having influenza-like illness.
